(19) United States
(12) Patent Application Publication
(10) Pub. No.: US 2013/0090342 A1
(43) Pub. Date: Apr. 11, 2013

(54) BIPHENYL SUBSTITUTED 1,3-DIHYDRO-BENZOIMIDAZOL-2-YLIDENEAMINE DERIVATIVES

(71) Applicants: Frederic Berst, Altkirch (FR); Pascal Furet, Thann (FR); Andreas Marzinzik, Weil (DE)

(72) Inventors: Frederic Berst, Altkirch (FR); Pascal Furet, Thann (FR); Andreas Marzinzik, Weil (DE)

(73) Assignee: NOVARTIS AG, Basel (CH)

(21) Appl. No.: 13/704,373

(22) PCT Filed: Jun. 16, 2011
(86) PCT No.: PCT/EP2011/060014

§ 371 Date: Dec. 14, 2012

Related U.S. Application Data
(60) Provisional application No. 61/355,838, filed on Jun. 17, 2010.

(51) Int. Cl. C07D 235/30; C07D 401/10; A61K 31/4439; C07D 401/12; C07D 405/12; A61K 45/06; C07D 403/10; C07D 401/14; C07D 403/04; A61K 31/501; C07D 403/14; C07D 401/04; A61K 31/4184; A61K 31/454

(52) U.S. Cl. CPC C07D 235/30; A61K 31/4184; C07D 401/10; A61K 31/4439; C07D 401/12; C07D 405/12; A61K 31/454; C07D 403/10; C07D 401/14; C07D 403/04; A61K 31/501; C07D 403/14; C07D 401/04; A61K 45/06
USPC 514/252.06; 548/307.4; 514/388; 546/273.4; 514/338; 548/304.7; 546/199; 514/322; 548/305.1; 544/238

(57) ABSTRACT

The invention relates to new derivatives of formula (I),

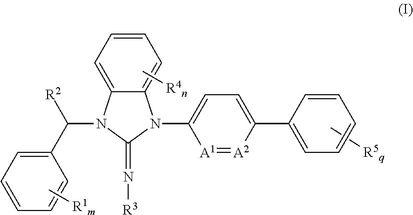

wherein the substituents are as defined in the specification; to processes for the preparation of such derivatives; pharmaceutical compositions comprising such derivatives; such derivatives as a medicament; such derivatives for the treatment of one or more IGF-1R mediated disorders or diseases.

### FIELD OF THE INVENTION

The invention relates to new derivatives of 1H-benzo[d]imidazol-2(3H)-imines; processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

### BACKGROUND OF THE INVENTION

Insulin-like growth factor (IGF-1) signaling is highly implicated in cancer, with the IGF-1 receptor (IGF-1R) as the predominating factor. IGR-1R is important for tumor transformation and survival of malignant cells, but is only partially involved in normal cell growth. Targeting of IGF-1R has been suggested to be a promising option for cancer therapy. (Larsson et al., Br. J. Cancer 92:2097-2101 (2005)).

WO 2005/097800 discloses certain 6,6-bicyclic ring substituted heterobicyclic derivatives having therapeutic activity as IGF-1R inhibitors. WO 2005/037836 discloses certain imidazopyrazine derivatives having therapeutic activity as IGF-1R inhibitors. WO2006/074991 discloses certain 1H-benzo[d]imidazol-2(3H)-imines having therapeutic activity as modulators of the SK-channels.

Because of the emerging disease-related roles of IGF-1R, there is a continuing need for compounds which may be useful for treating and preventing a disease which responds to inhibition of IGF-1R, particularly for compounds with improved efficacy, tolerability and/or selectivity. They should be well absorbed from the gastrointestinal tract, be metabolically stable and possess favourable pharmacokinetic properties. They should be non-toxic and demonstrate few side-effects. Furthermore, the ideal drug candidate will exist in a physical form that is stable, non-hygroscopic and easily formulated.

### SUMMARY OF THE INVENTION

The invention relates to new derivatives of 1H-benzo[d]imidazol-2(3H)-imines of formula (I)

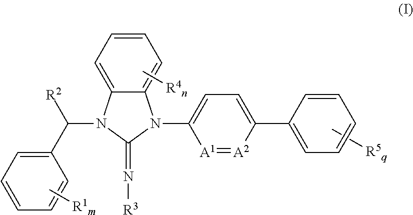

or a salt thereof, wherein R¹-R⁵, A¹, A², B, m, n, and q are defined below. The invention also relates to processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

### DETAILED DESCRIPTION OF THE INVENTION

The invention therefore provides in a first aspect a compound of formula (I),

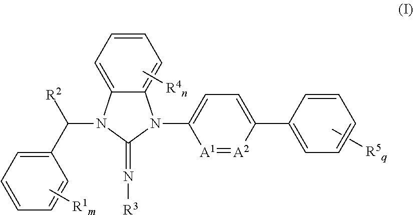

or a salt thereof, wherein
m represents 0, 1, 2, 3 or 4;
n represents 0, 1, 2, 3 or 4;
q represents 0, 1, 2, 3, 4 or 5;
A¹ represents N or CR⁶;
A² represents N or CR⁷;
R¹ represents halogen, C₁₋₇alkyl, C₁₋₇alkyoxy, halo-C₁₋₇alkyl or halo-C₁₋₇alkyoxy; and/or
R¹ represents, provided two substituents R¹ are in vicinal position, together with the carbon atoms to which they are attached a cyclic moiety, said moiety being (a) saturated or partly saturated, (b) contains 5-8 ring forming atoms, (c) contains 0-3 nitrogen atoms, 0-2 oxygen atoms, and 0-2 sulfur atoms, and (d) is unsubstituted or substituted, the substituents being selected from the group consisting of halogen, C₁₋₇alkyl, C₁₋₇alkyoxy, halo-C₁₋₇alkyl and halo-C₁₋₇alkyoxy;
R² represents hydrogen, halogen, C₁₋₇alkyl or halo-C₁₋₇alkyl;
R³ represents hydrogen, C₁₋₇alkyl, halo-C₁₋₇alkyl, C₁₋₇alkyl-carbonyl-C₁₋₇alkyl, halo-C₁₋₇alkyl-carbonyl-C₀₋₇alkyl, C₁₋₇alkoxy-carbonyl-C₀₋₇alkyl, halo-C₁₋₇alkoxy-carbonyl-C₀₋₇alkyl, C₃₋₆cycloalkyl, or halo-C₃₋₆cycloalkyl;
R⁴ represents halogen, C₁₋₇alkyl, C₁₋₇alkoxy, halo-C₁₋₇alkyl or halo-C₁₋₇alkoxy;
R⁵ represents a substituent different from hydrogen, said substituent (a) having 1-50 atoms selected from the group consisting of hydrogen, carbon, halogen and hetero atoms and (b) being bound via a single bond; and/or R⁵ represents, provided two substituents R⁵ are in vicinal position, together with the carbon atoms to which they are attached a cyclic moiety, said moiety being (a) saturated or partly saturated, (b) contains 5-8 ring forming atoms, (c) contains 0-3 nitrogen atoms, 0-2 oxygen atoms, and 0-2 sulfur atoms, (d) is unsubstituted or substituted by 1, 2 or 3 substituents, (e) said substituent having 1-50 atoms selected from the group consisting of hydrogen, carbon, halogen and hetero atoms, and (f) said substituent being bound via a single bond or double bond;
R⁶ represents, hydrogen, hydroxy, halogen, C₁₋₇alkyl, C₁₋₇alkyoxy, halo-C₁₋₇alkyl or halo-C₁₋₇alkyoxy;
R⁷ represents, hydrogen, hydroxy, halogen, C₁₋₇alkyl, C₁₋₇alkyoxy, halo-C₁₋₇alkyl or halo-C₁₋₇alkyoxy.

It has been found that the compounds of formula (I), described below, are potent inhibitors of the tyrosine kinase activity of the Insulin-like growth factor I receptor (IGF-IR) and inhibit IGF-1R-dependent cell proliferation. The presence of the substituents of the scaffold as defined below is considered important for the efficacy, tolerability and/or the selectivity of the compounds of the present invention as IGF-IR tyrosine kinase inhibitors and their potential to inhibit IGF-1R-dependent cell proliferation.

The compounds of the present invention are therefore potentially useful in the treatment of a wide range of disorders, particularly the treatment of proliferative diseases. The compounds of formula (I) therefore permit, for example, a therapeutic approach, especially for diseases in the treatment of which, and also for the prevention of which, an inhibition of the IGF-IR tyrosine kinase and/or of the IGF-1R-dependent cell proliferation shows beneficial effects. Such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas. Compounds of the invention show improved efficacy, tolerability and/or selectivity when compared to known IGF-1R inhibitors. Without being bound to theory, it is believed that several factors contribute to the improvements in efficacy and tolerability, for example increased metabolic stability and the reduced formation of multiple kinase-active metabolites. Although known compounds have been shown to produce desirable effects in in-vivo models through the inhibition of IGF-1 receptor activity, they have been found to undergo extensive metabolism. This not only limits the pharmacokinetic profile of such derivatives, but also generates metabolites, which show multiple potent kinase activities.

The invention may be more fully appreciated by reference to the following description, including the following glossary of terms and the concluding examples. As used herein, the terms “including”, “containing” and “comprising” are used herein in their open, non-limiting sense. Where the plural form is used for compounds, salts, and the like, this is taken to mean also a single compound, salt, or the like.

Unless specified otherwise, the term “compounds of the present invention” refers to compounds of formula (I) and subformulae thereof (add other additional genus structures as necessary), prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any asymmetric atom of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Substituents at atoms with unsaturated bonds may, if possible, be present in cis-(Z)- or trans-(E)-form. Particularly, R³ may be present in cis-form, trans-form or mixtures thereof.

Accordingly, as used herein, a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Compounds of the present invention are either obtained in the free form, as a salt thereof, or as prodrug derivatives thereof. When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released trans-port moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxylcarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present invention, including salts, hydrates and solvates thereof, may inherently or by design form polymorphs.

Compounds of the present invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the cocrystal former under crystallization conditions and isolating co-crystals thereby formed. Suitable co-crystal formers include those described in WO 2004/078163. Hence the invention further provides co-crystals comprising a compound of formula (I).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therepeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed. Pharmaceutically acceptable solvates in accordance with the invention include those wherein the solvent of crystallization may be isotopically substituted, e.g. D₂O, d₆-acetone, d₆-DMSO.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

As used herein, a carbon containing group, moiety or molecule contains 1 to 12, preferably 1 to 7, more preferably 1 to 4, most preferably 1 or 2, carbon atoms. Any non-cyclic carbon containing group or moiety with more than 1 carbon atom is straight-chain or branched. The prefix “lower” denotes a radical having 1 to 7, preferably 1 to 4 carbon atoms, the radicals in question being either unbranched or branched with single or multiple branching.

As used herein, the term “halogen” (or halo) refers to fluorine, bromine, chlorine or iodine, in particular fluorine, chlorine. Halogen-substituted groups and moieties, such as alkyl substituted by halogen (haloalkyl) can be mono-, poly- or per-halogenated.

As used herein, the term “hetero atoms” refers to atoms other than Carbon and Hydrogen, preferably nitrogen (N), oxygen (O) or sulfur (S), in particular nitrogen or oxygen.

As used herein, the term “alkyl” refers to a fully saturated branched or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, npentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. A substituted alkyl is an alkyl group containing one or more, such as one, two or three substituents as defined herein.

As used herein, the term “alkylene” refers to divalent alkyl group as defined herein above having 1 to 20 carbon atoms. It comprises 1 to 20 carbon atoms, Unless otherwise provided, alkylene refers to moieties having 1 to 16 carbon atoms, 1 to 10 carbon atoms, 1 to 7 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents as defined herein.

As used herein, the term “haloalkyl” refers to an alkyl as defined herein, which is substituted by one or more halo groups as defined herein. The haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Typically the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have 1-16, 1-10, 1-7, more preferably 1-4 carbon atoms. A substituted alkoxy is an alkoxy group containing one or more, such as one, two or three substituents as defined herein; preferably halo.

Similarily, each alkyl part of other groups like “alkylaminocrabonyl”, “alkoxyalkyl”, “alkoxylcarbonyl”, “alkoxy-carbonylalkyl”, “alkylsulfonyl”, “alkylsulfoxyl”, “alkylamino”, “haloalkyl” shall have the same meaning as described in the above-mentioned definition of “alkyl”.

As used herein, the term “cycloalkyl” refers to saturated or unsaturated monocyclic, bicyclic, tricyclic or spirocyclic hydrocarbon groups of 3-12 carbon atoms. Unless otherwise provided, cycloalkyl refers to cyclic hydrocarbon groups having between 3 and 9 ring carbon atoms or between 3 and 7 ring carbon atoms. A substituted cycloalkyl is a cycloylkyl group containing one or more substituents as defined herein. Preferably, a substituted cycloalkyl is a cycloalkyl group substituted by one, or two, or three, or more substituents independently selected from the group consisting of alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, and heterocyclyl. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

Similarily, each cycloalkyl part of other groups like “cycloalkyloxy”, “cycloalkoxyalkyl”, “cycloalkoxycarbonyl”, “cycloalkoxy-carbonylalkyl”, “cycloalkylsulfonyl”, “halocycloalkyl” shall have the same meaning as described in the above-mentioned definition of “alkyl”.

As used herein, the term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl. A substituted aryl is an aryl group containing one or more substituents as defined herein. Preferably, a substituted aryl is an aryl group substituted by 1-5 (such as one, or two, or three) substituents independently selected from the group consisting of alkyl, haloalkyl, cycloalkyl, halogen, hydroxy, alkoxy, acyl, alkyl-C(O)—O—, aryloxy, heteroaryloxy-, amino, thiol, alkylthio, arylthio-, nitro, cyano, carboxy, alkyl-O—C(O)—, carbamoyl, alkyl-S(O)—, sulfonyl, sulfonamido, aryl and heterocyclyl.

Similarily, each aryl part of other groups like “aryloxy”, “aryloxyalkyl”, “aryloxycarbonyl”, “aryloxy-carbonylalkyl” shall have the same meaning as described in the above-mentioned definition of “aryl”.

As used herein, the term “heterocyclyl” refers to a heterocyclic radical that saturated or partially saturated and is preferably a monocyclic or a polycyclic ring (in case of a polycyclic ring particularly a bicyclic, tricyclic or spirocyclic ring); and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl excludes heteroaryl. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like. A substituted heterocyclyl is a heterocyclyl group containing one or more substituents as defined herein. Preferably, a substituted heterocyclyl is an heterocyclyl group substituted by 1-5 (such as one, or two, or three) substituents independently selected from the group consisting of the substituents defined above for substituted alkyl and/or from one or more of the following substituents: alkyl, oxo (═O), thiono (═S), imino(═NH), imino-alkyl.

Similarily, each heterocyclyl part of other groups like “heterocyclyloxy”, “heterocyclyloxyalkyl”, “heterocyclyloxycarbonyl” shall have the same meaning as described in the above-mentioned definition of “heterocyclyl”.

As used herein, the term “heteroaryl” refers to a 5-14 membered monocyclic- or bicyclic- or tricyclic-aromatic ring system, having 1 to 8 heteroatoms. Typically, the heteroaryl is a 5-10 membered ring system (e.g., 5-7 membered monocycle or an 8-10 membered bicycle) or a 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl. The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinolyl, 1-, 3-, 4-, 5-, 6-, 7-, or 8-isoquinolyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4-aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl.

A substituted heteroaryl is a heteroyryl group containing one or more substituents as defined herein. Preferably, a substituted heteroyryl is a heteroyryl group substituted by 1-5 (such as one, or two, or three) substituents independently selected from the group consisting of the substituents defined above for substituted alkyl and/or from one or more of the following substituents: alkyl, oxo (═O), thiono (═S), imino(═NH), imino-alkyl.

Similarily, each heteroaryl part of other groups like “heteroaryloxy”, “heteroaryloxyalkyl”, “heteroaryloxycarbonyl” shall have the same meaning as described in the above-mentioned definition of “heteroaryl”.

As used herein, the term “substituted” or “a substituent different from hydrogen” refers to a moiety that is substituted by one or more, typically 1, 2, 3 or 4, covalently bound suitable non-hydrogen substituents; said substituent containing 1-50 atoms selected from the group consisting of hydrogen, carbon, halogen and hetero atoms. Preferably, non-hydrogen substituents are each independently selected from the group consisting of:
(a) halo, nitro, cyano;
(b) oxo (═O), carboxyl (COOH), formyl (CHO), carbamoyl (CONH₂);
(c) mercapto (SH), sulfinyl (S(O)), sulfonyl (S(O₂)), sulfoxy (S(O)), sulfamoyl (SO₂NH₂), sulfonamido (e.g. SO₂N(H)C₁₋₇alkyl);
(d) alkyl, cycloalkyl; aryl, heterocyclyl, heteroaryl;
(e) hydroxy, alkoxy, cycloalkoxy, aryloxy, heterocyclyoxy, heteroaryloxy;
(f) alkyl-S—, cycloalkyl-S—, aryl-S—; heterocyclyl-S—, heteroaryl-S—;
(g) cycloalkyl-alkyl, aryl-alkyl, heterocyclyl-alkyl, heteroaryl-alkyl;
(h) amino, alkylamino, dialkylamino, cycloylkylamino, arylamino, heterocyclyolamino, heteroarylamino;
(i) alkyl-C(O)—O—, cycloalkyl-C(O)—O—, aryl-C(O)—O—; heterocyclyl-C(O)—O—, heteroaryl-C(O)—O—;
(j) alkyl-O—C(O)—; cycloalkyl-O—C(O)—, aryl-O—C(O)—; heterocyclyl-O—C(O)—, heteroaryl-O—C(O)—;
(k) alkyl-C(O)—NH—, cycloalkyl-C(O)—NH—, aryl-C(O)—NH—; heterocyclyl-C(O)—NH—, heteroaryl-C(O)—NH—;
(l) alkyl-NH—C(O)—; cycloalkyl-NH—C(O)—, aryl-NH—C(O)—; heterocyclyl-NH—C(O)—, heteroaryl-NH—C(O)—;
   wherein each cycloalkyl, aryl, heterocyclyl, heteroaryl may be substituted with halogen, hydroxy, alkyl, alkoxy, haloalkyl, haloakloxy, cycloalkyl, amino, alkylamino, dialkylamino alkyl-C(O)—NH—, alkyl-NH—C(O)—, as defined herein and
   wherein each alkyl may be substituted with halogen, hydroxy, alkoxy, haloalkyl, haloakloxy, cycloalkyl, amino, alkylamino, dialkylamino alkyl-C(O)—NH—, alkyl-NH—C(O)—, as defined herein and wherein each sulfonyl, sulfoxy, sulfamoyl, sulfonamido may be substituted with alkyl, cycloalkyl, aryl, heterocyclyl, heteroaryl.

In preferred embodiments, which are preferred independently, collectively or in any combination or sub-combination, the invention relates to a compound of the formula (I), wherein the substituents are as defined herein.

The invention further relates to pharmaceutically acceptable prodrugs of a compound of formula (I). Particularly, the present invention also relates to pro-drugs of a compound of formula (I) as defined herein that convert in vivo to the compound of formula (I) as such.

The invention further relates to pharmaceutically acceptable metabolites of a compound of formula (I).

Various embodiments of the invention are described herein. It will be recognized that features specified in each embodiment may be combined with other specified features to provide further embodiments.

Consequently, in one embodiment, the invention provides a compound of the formula (I), or a salt thereof, depicted by formula (I-1)

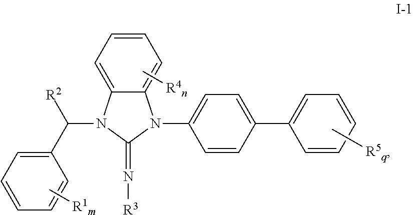

wherein the substituents are as defined herein.

In a further embodiment the invention provides a compound of the formula (I), or a salt thereof, depicted by formula (I-2)

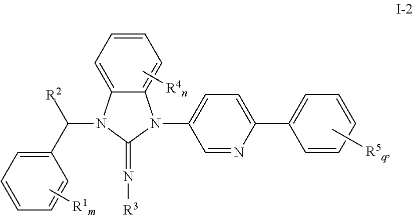

wherein the substituents are as defined herein.

In a further embodiment the invention provides a compound of the formula (I), or a salt thereof, depicted by formula (I-3)

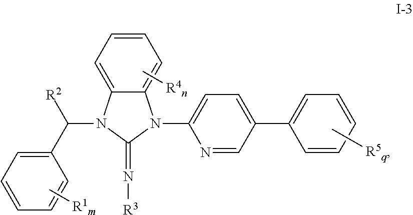

wherein the substituents are as defined herein.

In a further embodiment the invention provides a compound of the formula (I), or a salt thereof, depicted by formula (I-4)

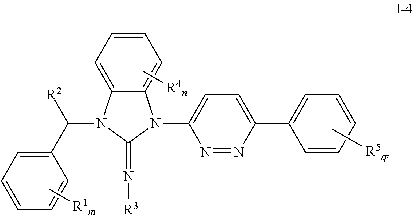

wherein the substituents are as defined herein.

In a further embodiment the invention provides a compound of the formula (I), or a salt thereof, depicted by formula (I-5)

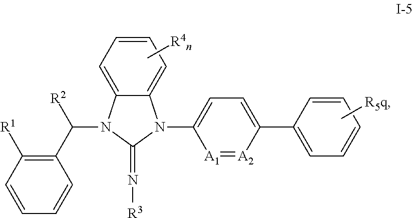

wherein the substituents are as defined herein.

In a further embodiment the invention provides a compound of the formula (I), or a salt hereof, depicted by formula (I-6)

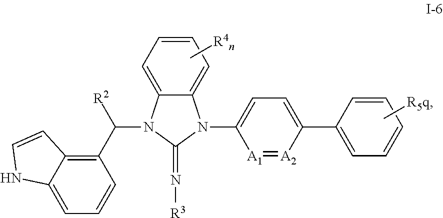

wherein the substituents are as defined herein.

In a further embodiment the invention provides a compound of the formula (I), or a salt thereof, depicted by formula (I-7)

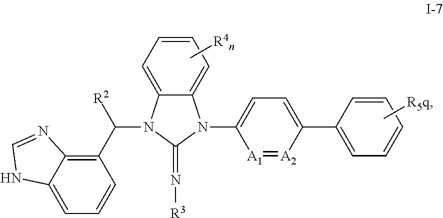

wherein the substituents are as defined herein.

In a further embodiment the invention provides a compound of the formula (I), or a salt thereof, depicted by formula (I-8)

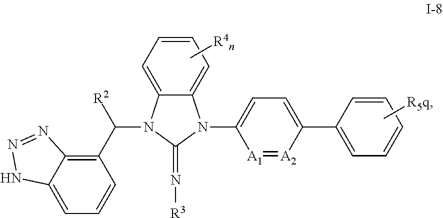

wherein the substituents are as defined herein.

In a further embodiment the invention provides a compound of the formula (I), or a salt thereof, depicted by formula (I-9)

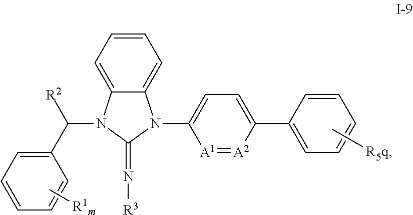

wherein the substituents are as defined herein.

In a further embodiment the invention provides a compound of the formula (I), or a salt thereof, depicted by formula (I-10)

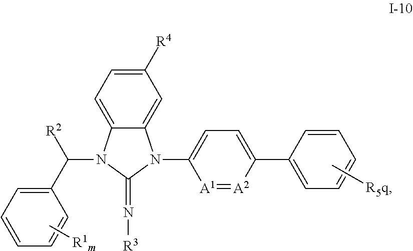

wherein the substituents are as defined herein.

In a further embodiment, m represents 0, 1, 2 or 3; particularly 1 or 2.

In a further embodiment, n represents 0, 1 or 2; particularly 0.

In a further embodiment, q represents 0, 1, 2, 3 or 4; particularly 1 or 2.

In a further embodiment, q represents 2, the substituents R⁵ being located in the 2- and 5-position.

In a further embodiment, q represents 1, the substituent R⁵ being located in the 2- or 3-position.

In a further embodiment, R¹ represents halogen; particularly fluoro or chloro.

In a further embodiment, R¹ represents, together with the phenyl ring, an unsubstituted or substituted indolyl, isoindolyl, indazolyl, benzimidazolyl, benztriazolyl, chinolinyl, isochinnolinyl, cinnolinyl, phtalazinyl, chinazolinyl, chinoxalinyl, naphtalenyl, tetrahydronaphtalenyl, indenyl, dihydro-indenyl, the substituents being selected from the group consisting of halogen; particularly fluoro or chloro.

In a further embodiment, R¹ represents, together with the phenyl ring, an unsubstituted or substituted indolyl, benzimidazolyl, benztriazolyl, the substituents being selected from the group consisting of fluoro and chloro.

In a further embodiment, R² represents hydrogen or C₁₋₇alkyl; particularly hydrogen.

In a further embodiment, R³ represents hydrogen, C₁₋₄alkyl which is optionally substituted by halo or C₁₋₄alkyloxy-carbonyl, C₃₋₆cycloalkyl which is optionally substituted by halo; particularly hydrogen, methyl, ethyl, iso-proyl, cyclopropyl, 2-fluoro-ethyl, methoxycarbonyl-methyl; particular preferably hydrogen.

In a further embodiment, R⁴ represents halogen, C₁ alkyl which is optionally substituted by halogen, C₁₋₄alkoxy which is optionally substituted by halogen; particularly fluoro, chloro, methyl, methoxy.

In a further embodiment, R⁵ represents a group -X′-R⁵′ wherein

X′ represents either a single bond or a linker selected from the group consisting of

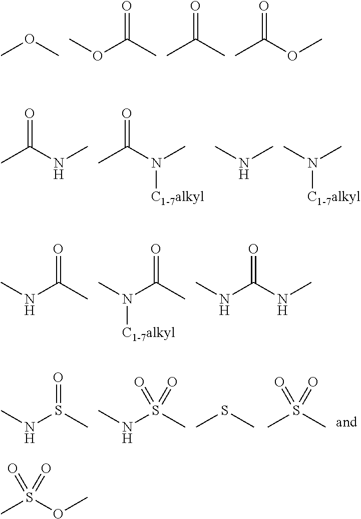

and
R⁵′ represents hydroxy, halo, cyano, carboxy (—CO₂H), aminocarbonyl (—CONH₂), amino, or optionally substituted C₁₋₇alkyl, optionally substituted C₃₋₁₂cycloalkyl, optionally substituted C₆₋₂₀aryl, optionally substituted heterocyclyl having 3-24 ring atoms, optionally substituted heteroaryl having 5-14 ring atoms, the optional substituents being selected from the group consisting of hydroxy, halo, cyano, carboxy, aminocarbonyl, amino, C₁₋₇alkylamino, di-(C₁₋₇alkyl)amino, C₁₋₇alkyl, C₁₋₇alkoxy, phenyl.

In a further embodiment, R⁵ represents a group -X′-R⁵′ wherein

X′ represents a linker selected from the group consisting of

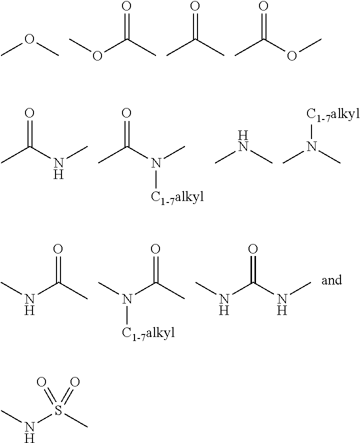

and
R⁵′ represents optionally substituted C₁₋₄alkyl, optionally substituted C₅₋₆cycloalkyl, optionally substituted C₆₋₁₀aryl, optionally substituted heterocyclyl having 4-10 ring atoms, optionally substituted heteroaryl having 5-10 ring atoms, the optional substituents being selected from the group consisting of hydroxy, halo, cyano, carboxy, amino-carbonyl, amino, C₁₋₅alkylamino, di-(C₁₋₅alkyl)amino, C₁₋₅alkyl, C₁₋₄alkoxy, phenyl.

In a further embodiment, R⁵ represents a group -X′-R⁵′ wherein

X′ represents a single bond and
R⁵′ represents, hydroxy, halo, cyano, carboxy, aminocarbonyl (CONH2), amino, C₁₋₇alkyl or substituted C₁₋₇alkyl, the substituents being selected from the group consisting of hydroxy, halo, amino, C₁₋₇alkylamino, C₁₋₇alkyloxy.

In a further embodiment, R⁵ represents methyl, methoxy, acetylamino (acetamide), chloro, cyano, trifluoromethyl, particularly acetamide.

In a further embodiment, R⁵ represents, together with the phenyl ring, an unsubstituted or substituted indolyl, isoindolyl, indazolyl, benzimidazolyl, benztriazolyl, chinolinyl, isochinnolinyl, cinnolinyl, phtalazinyl, chinazolinyl, chinoxalinyl, naphtalenyl, tetrahydronaphtalenyl, indenyl, dihydro-indenyl, the substituents being selected from the group consisting of hydroxy, halo, cyano, carboxy, aminocarbonyl, amino, C₁₋₇alkylamino, di (C₁₋₇alkyl)amino, C₁₋₇alkyl, C₁₋₇alkoxy, phenyl; particularly fluoro or chloro.

In a further embodiment, R⁵ represents, together with the phenyl ring, an unsubstituted or substituted indolyl, benzimidazolyl, benztriazolyl, the substituents being selected from the group consisting of hydroxy, halo, cyano, carboxy, amino-carbonyl, amino, C₁₋₅alkylamino, di (C₁₋₅alkyl)amino, C₁₋₅alkyl, C₁₋₄alkoxy, phenyl.

In a further embodiment, R⁶ represents hydrogen or C₁₋₄ alkyl; particularly hydrogen or methyl.

In a further embodiment, R⁷ represents hydrogen or C₁₋₄ alkyl; particularly hydrogen or methyl.

In a further embodiment, A¹ represents N, CH or CCH₃; particularly CH.

In a further embodiment, A² represents N, CH or CCH₃; particularly CH.

In a very particularly advantageous embodiment, the present invention relates to a compound of formula (I) mentioned in the examples below, or a salt, especially a pharmaceutically acceptable salt, thereof.

The invention relates in a second aspect to the manufacture of a compound of formula (I). The compounds of formula (I) or salts thereof are prepared in accordance with processes known per se (see references cited above), though not previously described for the manufacture of the compounds of the formula (I).

### General Reaction Processes:

(Method a1, a2)

In one embodiment, the invention relates to a process for manufacturing a compound of formula (I) wherein R³ represents hydrogen, said method comprising the step of reacting a compound of formula (II)

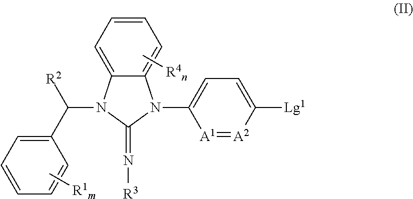

wherein the substituents are as defined above and Lg¹ represents a suitable leaving group, such as halogen (e.g. bromo or chloro), with a compound of formula (III),

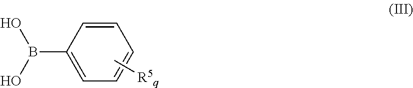

wherein the substituents are as defined above; optionally in the presence of one or more reaction aids, such as an inorganic base (e.g. Na₂CO₃, K₃PO₄) and a Pd catalyst (e.g. Pd(PPh₃)₄, Pd(PPh₃)₂Cl₂); optionally in the presence of one or more diluents, particularly polar solvents (e.g. dimethoxyethane, water) or apolar solvents (e.g. toluene).

This type of reaction is also known as Suzuki reaction; typical reaction conditions are known in the field and may applied to the present process. Particularly, a compound of formula (III) may be present as a boronic acid (as shown above) or as a boronic acid ester.

(Method b1)

In a further embodiment, the invention relates to a process for manufacturing a compound of formula (I) wherein R³ represents hydrogen, said method comprising the step of reacting a compound of formula (IX)

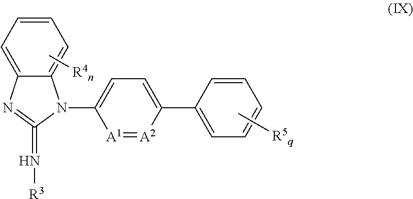

wherein the substituents are as defined above, with a compound of formula (V),

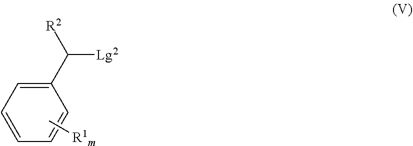

wherein the substituents are as defined above and Lg² represents a suitable leaving group, such as halo (e.g. chloro, bromo, iodo); optionally in the presence of one or more reaction aids, such as a base (e.g. Na₂CO₃) or an inorganic salt (e.g. KI); optionally in the presence of one or more diluents, particularly polar solvents (e.g. MeCN, Water).

This type of reaction is also known as alkylation reaction, typical reaction conditions are known in the field and may applied to the present process.

(Method c1)

In a further embodiment, the invention relates to a process for manufacturing a compound of formula (I) wherein R³ represents a substituent as defined herein except hydrogen, said method comprising the step of reacting a compound of formula (XIII)

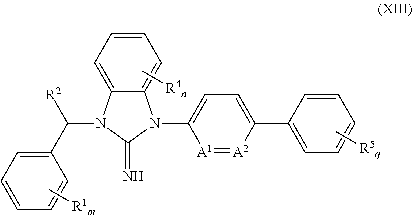

wherein the substituents are as defined herein, with a compound of formula (XII),

Lg⁵-R³′ (XIV)

wherein R³ represents as substituent as defined herein for R³ except hydrogen and Lg⁵ represents a suitable leaving group, such as halogen (e.g. chloro, fluoro, bromo); optionally in the presence of one or more reaction aids, such as an organic or inorganic base (e.g. NEt3, diisopropylethylamine, Na₂CO₃, Cs₂CO₃, K₂CO₃); optionally in the presence of one or more diluents, particularly one or more polar solvents (e.g. Ethyl acetate, dichloromethane, DMF, NMP, THF).

This type of reaction is also known as acylation (in case R³ represents alkyl-carbonyl) or alkylation (in case R³ represents alkyl) typical reaction conditions are known in the field and may applied to the present process.

### Starting Materials

New starting materials and/or intermediates, as well as processes for the preparation thereof, are likewise the subject of this invention. In a preferred embodiment, such starting materials are used and reaction conditions so selected as to enable the preferred compounds of the invention to be obtained.

Consequently, in one embodiment, the invention relates to a process for manufacturing a compound of formula (II),

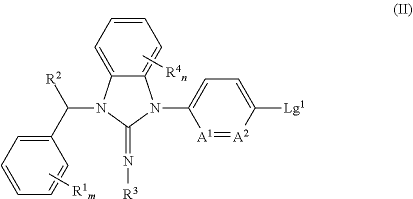

wherein the substituents are as defined above; said method comprising the step of reacting a compound of formula (IV)

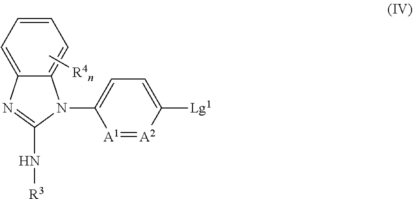

wherein the substituents are as defined above, with a compound of formula (V),

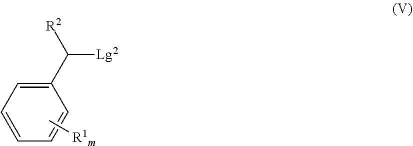

wherein the substituents are as defined above and Lg² represents a suitable leaving group, such as halo (e.g. chloro, bromo, iodo); optionally in the presence of one or more reaction aids, such as a base (e.g. Na₂CO₃) or an inorganic salt (e.g. KI); optionally in the presence of one or more diluents, particularly polar solvents (e.g. MeCN, Water).

This type of reaction is also known as alkylation reaction, typical reaction conditions are known in the field and may applied to the present process.

Starting materials of formula (V) are known or obtainable according to known processes; starting materials of formula (IV) are obtainable according to the processes as described herein.

In a further embodiment, the invention relates to a process for manufacturing a compound of formula (IV)

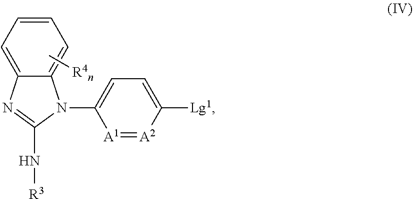

said method comprising the step of reacting a compound of formula (VI)

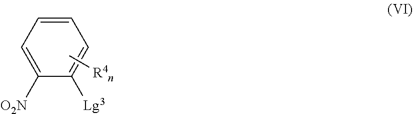

wherein the substituents are as defined above and Lg³ represents a suitable leaving group, particularly halo (e.g. fluoro); with a compound of formula (VII),

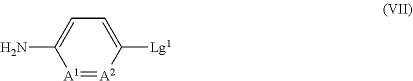

wherein the substituents are as defined above;
followed by the step of reacting the obtained intermediate with a reducing agent, such as hydrogen gas in the presence of a palladium (0) catalyst, or an organometallic salt, such as SnCl₂
followed by reacting the obtained intermediate with a compound of formula (VIII)

Lg⁴-CN (VIII)

wherein Lg⁴ represents a suitable leaving group, such as halogen (e.g. bromo); optionally in the presence of one or more diluents, particularly polar solvents (e.g. MeCN).

The above described first step is also known as aromatic nucleophilic substitution, the above described second step is known as reduction of a nitro to an amino group, the above described third step is known as a cyclisation reaction; typical reaction conditions for all steps are known in the field and may applied to the present process.

Starting materials are known or obtainable according to known processes; certain starting materials are new, obtainable according to the methods described herein and also subject of this invention.

In a further embodiment, the invention relates to a process for manufacturing a compound of formula (IX)

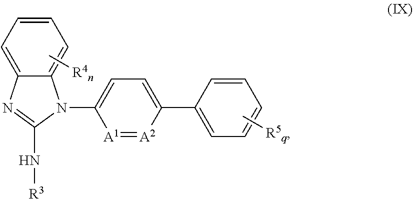

said method comprising the step of reacting a compound of formula (IV)

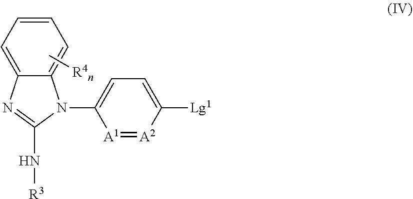

wherein the substituents are as defined above, and Lg¹ represents a suitable leaving group, particularly halo (e.g. bromo, chloro, iodo); with a compound of formula (III),

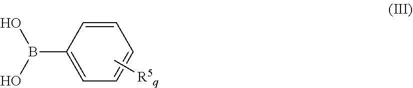

wherein the substituents are as defined above,
wherein the substituents are as defined above; optionally in the presence of one or more reaction aids, such as an inorganic base (e.g. Na₂CO₃, K₃PO₄) and a Pd catalyst (e.g. Pd(PPh₃)₄, Pd(PPh₃)₂Cl₂); optionally in the presence of one or more diluents, particularly polar solvents (e.g. dimethoxyethane, water) or apolar solvents (e.g. toluene).

This type of reaction is also known as Suzuki reaction; typical reaction conditions are known in the field and may applied to the present process. Particularly, a compound of formula (III) may be present as a boronic acid or as a boronic acid ester.

Starting materials of formula (VIII) are known or obtainable according to known processes. Starting materials of formula (IV) are obtainable according to the processes described herein.

In one embodiment, the invention relates to a process for manufacturing a compound of formula (IX)

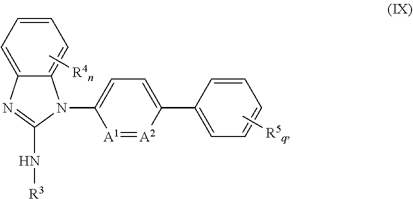

wherein the substituents are as defined above and R³ represents hydrogen, said method comprising the step of reacting a compound of formula (X)

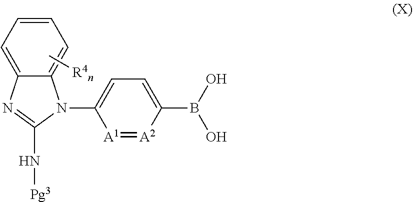

wherein the substituents are as defined above, and Pg³ represents a suitable protecting group, particularly BOC, with a compound of formula (XI),

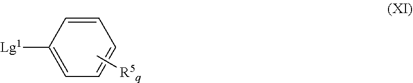

wherein the substituents are as defined above; optionally in the presence of one or more reaction aids, such as an inorganic base (e.g. Na₂CO₃, K₃PO₄) and a Pd catalyst (e.g. Pd(PPh₃)₄, Pd(PPh₃)₂Cl₂); optionally in the presence of one or more diluents, particularly polar solvents (e.g. dimethoxyethane, water) or apolar solvents (e.g. toluene).

This type of reaction is also known as Suzuki reaction; typical reaction conditions are known in the field and may applied to the present process. Particularly, a compound of formula (III) may be present as a boronic acid (as shown above) or as a boronic acid ester. Starting materials of formula (XI) and (X) are known or readily obtainable.

Further starting materials used in the above described processes are known, capable of being prepared according to known processes, or commercially obtainable; in particular, they can be prepared using processes as described in the examples. In the preparation of starting materials, existing functional groups which do not participate in the reaction may be protected. Preferred protecting groups, their introduction and their removal are described above or in the examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

### Protecting Groups:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions. Particularly, any amino group (—NH2 or —NH) may be protected by a BOC group if the reaction takes place in basic conditions (e.g. suzuki-type reactions); such BOC group may be removed using a strong acid. Further, any amino group may be protected by an FMOC group if the reaction takes place in acidic conditions; such FMOC group may be removed using a strong acid.

### Additional Process Steps:

In the methods described herein, (a) a compound of formula (I) obtained may be converted into another compound of formula (I), (b) a free compound of formula (I) may be converted into a salt, (c) a salt of a compound of formula (I) may be converted into the free compound or another salt, and/or (d) a mixture of isomeric compounds of formula (I) may separated into the individual isomers. Particularly, the conversion of R⁵ to another R⁵ (e.g. by reduction, substitution and/or oxidation) is considered such conversion (a) as described above. Further the conversion of R³=hydrogen into another substituent R³ is considered such conversion (a).

### General Process Conditions:

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralising agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C. or at the boiling point of the solvent used, under atmospheric pressure or in a closed vessel, if need be under pressure, and/or in an inert, for example an argon or nitrogen, atmosphere.

The invention relates also to those embodiments of the process in which one starts from a compound obtainable at any stage as an intermediate and carries out the missing steps, or breaks off the process at any stage, or forms a starting material under the reaction conditions, or uses said starting material in the form of a reactive derivative or salt, or produces a compound obtainable by means of the process according to the invention under those process conditions, and further processes the said compound in situ. In the preferred embodiment, one starts from those starting materials which lead to the compounds described hereinabove as preferred.

The compounds of formula (I) (or N-oxides thereof), including their salts, are also obtainable in the form of hydrates, or their crystals can include for example the solvent used for crystallisation (present as solvates).

In the preferred embodiment, a compound of formula (I) is prepared according to the processes and process steps defined in the Examples.

The invention relates in a third aspect to the use of compounds of the present invention as pharmaceuticals. Particularly, the compounds of formula (I) have valuable pharmacological properties, as described hereinbefore and hereinafter.

The invention thus provides:
a compound of the formula (I) as defined herein, as pharmaceutical/for use as pharmaceutical;
a compound of the formula (I) as defined herein, as medicament/for use as medicament;
a compound of the formula (I) as defined herein, for the treatment of/for use in the treatment of an IGF-1R mediated disorders or diseases;
a compound of the formula (I) as defined herein, for the inhibition of the IGF-IR tyrosine kinase;
a compound of the formula (I) as defined herein, for the treatment of/for use in the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of/for the manufacture of a medicament for the treatment of an IGF-1R mediated disorder or disease;
the use of a compound of formula (I) as defined herein for the inhibition of the IGF-IR tyrosine kinase;
the use of a compound of formula (I) as defined herein, for the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of a disorder or disease selected from acute lung injury and pulmonary fibrosis;
a method of modulating IGF-1R activity in a subject, comprising the step of administering to a subject a therapeutically effective amount of a compound of formula (I) as definded herein;
a method for the treatment of an IGF-1R mediated disorder or disease comprising the step of administering to a subject a therapeutically effective amount of a compound of formula (I) as defined herein;
a method for inhibition IGF-1R in a cell, comprising contacting said cell with an effective amount of a compound of formula (I) as defined herein.

As used herein term “a therapeutically effective amount” of a compound of the present invention refers to an amount of the compound of formula (I) that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by IGF-1R, or (ii) associated with IGF-1R activity, or (iii) characterized by activity (normal or abnormal) of IGF-1R; or (2) reducing or inhibiting the activity of IGF-1R; or (3) reducing or inhibiting the expression of IGF-1R. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of IGF-1R; or at least partially reducing or inhibiting the expression of IGF-1R. The meaning of the term “a therapeutically effective amount” as illustrated in the above embodiment for IGF-1R also applies by the same means to any other relevant proteins/peptides/enzymes. An “effective amount” may be determined empirically and in a routine manner, in relation to the stated purpose. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “administration” or “administering” of the subject compound means providing a compound of formula (I) and prodrugs thereof to a subject in need of treatment. Administration “in combination with” one or more further therapeutic agents includes simultaneous (concurrent) and consecutive administration in any order, and in any route of administration.

The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), adrenocotical carcinoma (ACC), medulloblastoma and desmoid.

As used herein, the term “IGF-1R mediated disease” includes but is not limited to, multiple myeloma, neuroblastoma, synovial, hepatocellular, Ewing's Sarcoma, adrenocotical carcinoma (ACC), or a solid tumor selected from osteosarcoma, melanoma, tumor of breast, renal, prostate, colorectal, thyroid, ovarian, pancreatic, lung, uterine or gastrointestinal tumor.

It was further found that compounds of formula (I) are also useful in the treatment of acute lung injury and pulmonary fibrosis.

The invention provides in further embodiments methods to treat, ameliorate or prevent a condition which responds to inhibition of IGF-1R in a mammal suffering from said condition, comprising administering to the mammal a therapeutically effective amount of a compound of formula (I) as defined herein, and optionally in combination with a second therapeutic agent. The compounds of the invention may be administered, for example, to a mammal suffering from an autoimmune disease, a transplantation disease, an infectious disease or a cell proliferative disorder. In particular examples, the compounds of the invention may be used alone or in combination with a chemotherapeutic agent to treat a cell proliferative disorder.

In a further embodiment, the invention relates to a process or a method for the treatment of one of the pathological conditions mentioned hereinabove, especially a disease which responds to an inhibition of the IGF-IR tyrosine kinase or of the IGF-1R-dependent cell proliferation, especially a corresponding neoplastic disease. The compounds of formula (I), or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a compound of the present invention.

In a further embodiment, the invention relates to the use of a compound of formula (I), or a pharmaceutically acceptable salt thereof, especially a compound of formula (I) which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, preferably a disease which responds to an inhibition of the IGFIR tyrosine kinase or of the IGF-1R-dependent cell proliferation, especially a neoplastic disease, in particular if the said disease responds to an inhibition of the IGF-IR tyrosine kinase or of the IGF-1R-dependent cell proliferation.

In a further embodiment, the invention relates to the use of a compound of formula (I), or a pharmaceutically acceptable salt thereof, especially a compound of formula (I) which is said to be preferred, or a pharmaceutically acceptable salt thereof, for the preparation of a pharmaceutical composition for the therapeutic and also prophylactic management of one or more of the diseases mentioned hereinabove, especially a neoplastic disease, in particular if the disease responds to an inhibition of the IGF-IR tyrosine kinase or of the IGF-1R-dependent cell proliferation.

The invention relates in a fourth aspect to pharmaceutical compositions comprising a compound of the present invention.

The invention thus provides
a pharmaceutical composition comprising (i.e. containing or consisting of) a compound of formula (I) as defined herein and one or more carriers/excipients;
a pharmaceutical composition comprising a therapeutically effective amount of a compound of formula (I) as defined herein, and one or more pharmaceutically acceptable carriers/excipients.

As used herein, the term “pharmaceutically acceptable carrier” includes any and all solvents, dispersion media, coatings, surfactants, antioxidants, preservatives (e.g., anti-bacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Suitable excipients/carriers may be any solid, liquid, semi-solid or, in the case of an aerosol composition, gaseous excipient that is generally available to one of skill in the art. Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The dosage of the active ingredient depends upon the disease to be treated and upon the species, its age, weight, and individual condition, the individual pharmacokinetic data, and the mode of administration The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %. Unit dose forms are, for example, coated and uncoated tablets, ampoules, vials, suppositories or capsules. Examples are capsules containing from about 0.05 g to about 1.0 g of active substance.

Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the compound of formula (I) alone or, preferably, together with a pharmaceutically acceptable carrier.

Pharmaceutical compositions comprising a compound of formula (I) as defined herein in association with at least one pharmaceutical acceptable carrier (such as an excipient and/or diluent) may be manufactured in conventional manner, e.g. by means of conventional mixing, granulating, coating, dissolving or lyophilising processes.

In a further embodiment, the invention relates to a pharmaceutical composition for administration to a warm-blooded animal, especially humans or commercially useful mammals suffering from a disease which responds to an inhibition of the IGF-IR tyrosine kinase or of the IGF-1R-dependent cell proliferation, comprising an effective quantity of a compound of formula (I) for the inhibition of the IGF-IR tyrosine kinase or of the IGF-1R-dependent cell proliferation, or a pharmaceutically acceptable salt thereof, together with at least one pharmaceutically acceptable carrier.

In a further embodiment, the invention relates to a pharmaceutical composition for the prophylactic or especially therapeutic management of neoplastic and other proliferative diseases of a warm-blooded animal, especially a human or a commercially useful mammal requiring such treatment, especially suffering from such a disease, comprising as active ingredient in a quantity that is prophylactically or especially therapeutically active against said diseases a new compound of formula (I), or a pharmaceutically acceptable salt thereof, is likewise preferred.

The invention relates in a fifth aspect to combinations comprising a compound of formula (I) and one or more additional active ingredients.

The invention thus provides
a combination in particular a pharmaceutical combination, comprising a therapeutically effective amount of a compound of formula (I) and one or more therapeutically active agents, particularly antiproliferative agents;
a combined pharmaceutical composition, adapted for simultaneous or sequential administration, comprising a therapeutically effective amount of a compound of formula (I) as defined herein; therapeutically effective amount(s) of one or more combination partners, particularly antiproliferative agents; one or more pharmaceutically acceptable excepients;
a combined pharmaceutical composition as defined herein (i) as pharmaceutical, (ii) for use in the treatment of a IGF-1R mediated disease, (iii) in a method of treatment of a IGF-1R mediated disease.

As used herein, the term “combination” refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “coadministration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “antiproliferative agent” includes, but are not limited to, aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, compounds decreasing the lipid kinase activity, e.g. PI3 kinase inhibitors, antineoplastic antimetabolites, platin compounds, compounds decreasing the protein kinase activity, eg mTOR inhibitors, Raf inhibitors, MEK inhibitors, and further anti-angiogenic compounds, gonadorelin agonists, anti-androgens, bengamides, bisphosphonates and trastuzumab, radiotherapy.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETENT™.

A combination of the invention comprising an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™ Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOLT™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™ Mitoxantrone can be administered, e.g., in the form as it is marketed, e.g. under the trademark NOVANTRONT™.

The term “lipid kinase inhibitors' relates to PI3 kinase inhibitors, PI4 kinase inhibitors, Vps34 inhibitors. Specific examples include: NVP-BEZ235, NVP-BGT226, NVP-BKM120, AS-604850, AS-041164, AS-252424, AS-605240, GDC0941, PI-103, TGX221, YM201636, ZSTK474, examples described in WO 2009/080705 and US 2009/163469.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™.

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®) and rofecoxib (Vioxx®).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), and c-Src and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula (I)), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin, described by M. S. O'Reilly et al, Cell 79, 1994, 315-328; and Endostatin, described by M. S. O'Reilly et al, Cell 88, 1997, 277-285; sorefanib (Nexavar), Sutent (sunitinib), BAY 43-9006.

Compounds which decrease the activity of EGF are especially compounds which inhibit the EGF receptors, especially the tyrosine kinase activity of the EGF receptors, and compounds binding to EGF, and are in particular those compounds generically and specifically disclosed in WO 97/02266 (describing compounds of formula (I)V), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980. Specific EGF receptor inhibitor examples include, but not limited to; Tarceva (erlotinib), Iressa (Gefitinib), Tywerb (lapatanib). Erbitux (cetuximab), Avastin (bevacizumab), Herceptin (trastuzamab), Rituxan (rituximab), Bexxar (tositumomab), panitumumab.

Compounds which decrease the activity of c-Src include, but are not limited to, compounds inhibiting the c-Src protein tyrosine kinase activity as defined below and to SH2 interaction inhibitors such as those disclosed in WO97/07131 and WO97/08193; compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

Compounds which decrease the activity of Raf kinases include, but are not limited to: Raf265, sorefanib, BAY 43-9006.

Compounds which inhibit downstream effectors of Raf kinases, such as MEK. Examples of MEK inhibitors include; PD 98059, AZD6244 (ARRY-886), CI-1040, PD 0325901, u0126.

Anti-angiogenic compounds having another mechanism of action than decreasing the protein kinase activity include, but are not limited to e.g. thalidomide (THALOMIDT™), SU5416, and celecoxib (Celebrex™)

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties and includes, but is not limited to the compounds generically and specifically disclosed in WO00/29382, preferably to ex. 1 of WO00/29382.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™. “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETAT™.

“Trastuzumab” can be administered, e.g., in the form as it is marketed, e.g. under the trademark HERCEPTIN™.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of formula (I), can be prepared and administered as described in the art such as in the documents cited above.

The following Examples serve to illustrate the invention without limiting its scope. Abbreviations used are those conventional in the art or the following:

| | |
|---|---|
| AcOEt | Ethyl acetate |
| amu | Atomic mass units |
| Boc | Tert-Butoxycarbonyl |
| Boc₂O | Di-tert-butyl dicarbonate |
| CH2Cl2 | dichloromethane |
| DCE | 1,2-dichloroethane |
| DIBAL-H | Diisobutylaluminium hydride |
| DIC | Diisopropyl carbodiimide |
| DIPEA | Diisopropylethylamine |
| DMAP | N,N-Dimethylpyridin-4-amin |
| DME | 1,2-Dimethoxyethane |
| DMF | N,N-Dimethylformamide |
| dppf | 1,1′-bis(diphenylphosphanyl) |
| | ferrocene |
| HATU | (2-(7-Aza-1H-benzotriazole- |
| | 1-yl)-1,1,3,3-tetramethyl |
| | uroniumhexafluorophos- |
| | phate) |
| LiAlH4 | Lithium-aluminium hydride |
| mCPBA | m-chloroperbenzoic acid |
| MeCN | Acetonitrile |
| MeOH | Methanol |
| Na2SO4 | Sodium sulfate |
| NaOH | Sodium hydroxyde |
| NEt₃ | Triethylamine |
| NH4OH | Aqueous ammonia solution, |
| | 30% |
| NMP | N-methylpyrrolidinone |
| Pd(Ph3P)4 | Tetra- |
| | kis(triphenylphos- |
| | phane)palladium(0) |
| Pd/C | Palladium on carbon |
| PTFE | Polytetrafluorethylene, |
| | Teflon |
| Rac-BINAP | rac-2,2′-bis(diphenyl- |
| | phosphino)-1,1′-binaphthyl |
| rt | room temperature |
| TEA | triethylamine |
| TFA | Trifluoroacetic acid |
| THF | Tetrahydrofuran |
| t_{R} | Retention time |

### I Analytical Methods

Temperatures are measured in degrees Celsius.

Nuclear magnetic resonance spectra were recorded on a Bruker spectrometer at 400 mHz and at room temperature.

The following HPLC, MS and HPLC/MS methods are used in the preparation of the Intermediates and Examples:

### H PLC/MS Method A

Instrument: Waters Acquity Ultra Performance LC system, Waters 2996 photodiode array UV detector, Water SQ MS detector (range: 130-750 amu; cone: +10V and −30V), column oven temperature+40° C.

### Column: Acquity UPLC BRH C18 1.7 um, 2.1*50 mm

Flow rate: 0.7 mL/min
Eluent: A: water+0.1% formic acid; B: acetonitrile+0.1% formic acid

### Gradient:

| Time (min) | % B in A |
|---|---|
| 0 | 5 |
| 2 | 100 |

### HPLC/MS Method C

Instrument: Waters Acquity Ultra Performance LC system, Waters 2996 photodiode array UV detector, Water SQ MS detector (range: 130-750 amu; cone: +10V and -30V), column oven temperature+40° C.

### Column: Acquity UPLC BRH C18 1.7 um, 2.1*50 mm

Flow rate: 0.7 mL/min
Eluent: A: water+0.1% formic acid; B: acetonitrile+0.1% formic acid

### Gradient:

| Time (min) | % B in A |
|---|---|
| 0 | 50 |
| 1 | 100 |
| 2 | 100 |

### HPLC/MS Method D

Instrument: Waters Acquity Ultra Performance LC system, Waters 2996 photodiode array UV detector, Water SQ MS detector (range: 130-750 amu; cone: +10V and −30V), column oven temperature+40° C.

### Column: Acquity UPLC BRH C18 1.7 um, 2.1*50 mm

Flow rate: 0.7 mL/min
Eluent: A: water+0.1% formic acid; B: acetonitrile+0.1% formic acid

### Gradient:

| Time (min) | % B in A |
|---|---|
| 0 | 20 |
| 1 | 25 |
| 4.2 | 90 |
| 4.3 | 100 |

### HPLC Method E

Instrument: Waters HPLC system, Water 2545 binary gradient module, Waters 2996 photodiode array UV detector, Waters 2767 Auto Sampler/Fraction Collector.
Column: sunfire Prep C18 OBD 5 um, 30*100 mm.
Flow rate: 30 mL/min
Generic eluents Eluent: A: water+0.1% TFA; B: acetonitrile+0.1% TFA
Generic gradient from 0% B in A to 100% B in A over 20 minutes.

The generic conditions were used unless specified in the experimental part for individual intermediates/examples.

### HPLC/MS Method F

Instrument: Agilent 1100 LC chromatography system with Micromass ZMD MS detection (range: 100-900 amu; cone: +25V), column oven temperature+50° C.
Column: Ascentis Express C18 2.1 mm×30 mm 2.7u particles
Eluent: A: (water+0.1% TFA)/(acetonitrile+0.1% TFA): 95/5; B: acetonitrile+0.1% TFA

### Gradient:

| Time (min) | Flow rate | % B in A |
|---|---|---|
| 0 | 1.4 | 5 |
| 3.7 | 1.4 | 95 |
| 4.4 | 2.4 | 95 |

### HPLC/MS Method G

Instrument: Agilent 1100 LC chromatography system with Micromass ZMD MS detection (range: 100-900 amu; cone: +25V), column oven temperature+50° C.
Column: Waters XTerra C-18 column (3×30 mm; 2.5 um particle size)
Flow rate: 0.7 mL/min
Eluent: A: (water+0.1% TFA)/(acetonitrile+0.1% TFA): 95/5; B: acetonitrile+0.1% TFA

### Gradient:

| Time (min) | % B in A |
|---|---|
| 0 | 5 |
| 0.5 | 5 |
| 3.5 | 95 |
| 4.5 | 95 |

### II Chemical Synthesis—Intermediates

### Intermediate A: 1-(4-Bromo-phenyl)-1H-benzoimidazol-2-ylamine

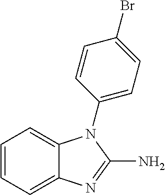 [SMILES: Nc1nc2ccccc2n1-c1ccc(Br)cc1]

N-(4-Bromo-phenyl)-benzene-1,2-diamine (step A.1, 2900 mg, 11.02 mmol) and cyanic bromide (2335 mg, 22.04 mmol) were dissolved in acetonitrile (51 mL) and water (3.5 mL). The resulting mixture was stirred at rt for 18 h. The medium was evaporated to dryness and the residual purple oil was diluted with 1M aqueous NaOH (50 mL) and CH2Cl2 (50 mL). The mixture was shaken vigorously and sonicated to dissolve all the solids. The phases were separated. The aqueous phase was extracted with CH2Cl2 (2×50 mL) and the combined organics were dried over Na₂SO₄ and concentrated to a crude red solid. The solid was purified by chromatography on silica gel using a 10% to 100% gradient of eluent B (MTBE+5% [7N NH₃ in MeOH]) in eluent A (DCM/Heptane:1/4). Product-containing fractions were pooled and evaporated to afford the title compound as a red solid, 2888 mg (91%). ¹H NMR (400 MHz, CDCl3) d ppm 4.98 (s, 2H) 6.96 (d, 1H) 7.03 (m, 1H) 7.16 (m, 1H) 7.36 (m, 2H) 7.44 (d, 1H) 7.72 (m, 2H).

### Step A.1: N-(4-Bromo-phenyl)benzene-1,2-diamine

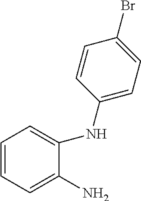 [SMILES: Nc1ccccc1Nc1ccc(Br)cc1]

A mixture of (4-Bromo-phenyl)-(2-nitro-phenyl)-amine (step A.2, 1465 mg, 5.00 mmol) and tin(II) chloride dihydrate (5639 mg, 24.99 mmol) in EtOH (17 mL) was heated to 70° C. for 170 minutes. The medium was then evaporated to dryness. The resulting crude oil was dissolved in EtOAc (30 ml) and treated with NaOH 2M (40 ml). The biphasic system was vigorously stirred for 20 minutes to allow complete precipitation of the tin salts (aqueous phase pH: 9). The medium was then filtered over diatomaceous earth through a sintered funnel and copiously washed with EtOAc. The biphasic filtrate was decanted, the organic layer was washed with brine (50 ml) and the organic finally dried over Na2SO4. Evaporation of the volatiles yielded a crude oil which crystallized on standing. The title product was obtained without need for further purification, 1279 mg (97%). HPLC/MS (method A) t_{R} 1.46 min, M+H 263-265.

### Step A.2: (4-Bromo-phenyl)-(2-nitro-phenyl)amine

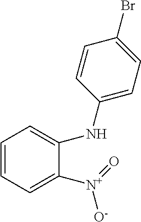 [SMILES: O=[N+]([O-])c1ccccc1Nc1ccc(Br)cc1]

A mixture of 2-Fluoro-1-nitro-benzene (749 μl, 7.09 mmol), p-bromoaniline (1219 mg, 7.09 mmol) and TEA (988 μl, 7.09 mmol) was heated to 150° C. for 240 minutes under microwave irradiation. The resulting red solid was triturated for 15 minutes in 30 ml 1M HCl and 20 ml water. The resulting suspension was then filtered to afford the title compound as a bright red solid, 1465 mg (70%), which did not require further purification. HPLC/MS (method A) t_{R} 1.78 min, M+H 293-295.

### Intermediate B: 1-(6-Chloro-pyridin-3-yl)-1H-benzoimidazol-2-ylamine

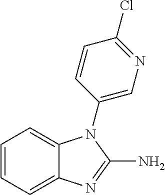 [SMILES: Nc1nc2ccccc2n1-c1ccc(Cl)nc1]

N-(6-Chloro-pyridin-3-yl)-benzene-1,2-diamine (step B.1, 4.7 g, 21.40 mmol) and cyanic bromide (4.6 g, 42.6 mmol) were dissolved in acetonitrile (107 mL) and water (7 mL). The resulting mixture was stirred at rt for 16 h. The medium was evaporated to dryness and the residual purple oil was diluted with 1M aqueous NaOH (100 mL) and CH2Cl2 (100 mL). The mixture was shaken vigorously and sonicated to dissolve all the solids. The phases were separated. The aqueous phase was extracted with CH₂Cl₂ (2×100 mL) and the combined organics were dried over Na2SO4 and concentrated to a crude red solid. The solid was purified by two consecutive chromatographies on silica gel using a 10% to 100% gradient of eluent B (AcOEt+1% 7N NH₃ in MeOH) in eluent A (Heptane), leading to the title compound as a grey solid, 4.2 g (80%). HPLC/MS (method A) t_{R} 0.72 minute, M+H 245. 1H NMR (DMSO-d₆) Ppm 6.46 (s, 2H) 6.80-6.93 (m, 2H) 7.03 (ddd, 1H) 7.23 (d, 1H) 7.77 (d, 1H) 8.04 (dd, 1H) 8.59 (d, 1H)

### Step B.1: N-(6-Chloro-pyridin-3-yl)-benzene-1,2-diamine

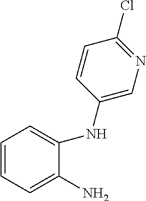 [SMILES: Nc1ccccc1Nc1ccc(Cl)nc1]

A mixture of (6-Chloro-pyridin-3-yl)-(2-nitro-phenyl)-amine (step B.2, 6.5 g, 26 mmol) and tin(II) chloride dihydrate (30 g, 130 mmol) in EtOH (87 mL) was heated to 70° C. for 120 minutes. The medium was then evaporated to dryness. The resulting crude oil was dissolved in EtOAc (100 ml) and treated with NaOH 1M (100 ml). The biphasic system was vigorously stirred for 20 minutes to allow complete precipitation of the tin salts. The medium was then filtered over diatomaceous earth through a sintered funnel and copiously washed with EtOAc. The biphasic filtrate was decanted, the organic layer was washed with brine (300 ml) and the organic finally dried over Na2SO4. Evaporation of the volatiles yielded the title product, 4.7 g (82%). HPLC/MS (method A) t_{R} 1.08 min, M+H 220.

### Step B.2: (6-Chloro-pyridin-3-yl)-(2-nitro-phenyl)-amine

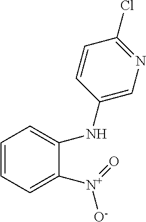 [SMILES: O=[N+]([O-])c1ccccc1Nc1ccc(Cl)nc1]

A mixture of 2-chloro-5-iodopyridine (12.0 g, 48.6 mmol), 2-nitroaniline (6.7 g, 48.6 mmol), cesium carbonate (79.2 g, 243.1 mmol), palladium diacetate (327 mg, 1.5 mmol), triethylamine (6.8 mL, 48.6 mmol) and rac-BINAP (936 mg, 1.5 mmol) in toluene (243 mL) under argon was heated to reflux for 48 hours. The reaction mixture was then diluted in 300 mL of AcOEt, filtered over a pad of celite and concentrated under reduced pressure to give a dark red solid. The red solid was triturated in 30 mL of heptane/ether (2/1), the precipitate was filtered, washed with heptane, and dried to afford the title compound as a red solid, 4.2 g. The trituration filtrate was concentrated to a red oil which was purified by chromatography on silica gel using a 5% to 100% gradient of eluent B (AcOEt+1% NH4OH) in eluent A (Heptane). Product-containing fractions were pooled and evaporated to furnish another 2.3 g of title compound which was combined with the solid from trituration, to afford the title compound as a red solid, 6.5 g (53%). HPLC/MS (Method A) t_{R} 1.41 minute, M+H 249.9-251.9.

### Intermediate C: 1-(6-Chloro-pyridin-3-yl)-6-fluoro-1H-benzoimidazol-2-ylamine

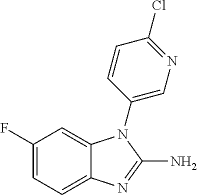 [SMILES: Nc1nc2ccc(F)cc2n1-c1ccc(Cl)nc1]

To a solution of N*2*-(6-Chloro-pyridin-3-yl)-4-fluoro-benzene-1,2-diamine (step C.1, 27.7 g, 117 mmol) in acetonitrile (217 ml) and water (16.32 ml) was added cyanogen bromide (12.35 g, 117 mmol) at 0° C. After 10 minutes the mixture was allowed to warm up to rt and the reaction mixture was stirred for 16 hours. Additional cyanogen bromide (2.469 g, 23.31 mmol) was then added to the mixture. After another hour of stirring at rt, the reaction mixture was concentrated under reduced pressure to a dark oil. This crude was taken up in AcOEt (500 mL) and washed with 1N NaOH (2×200 mL). The aqueous layer was extracted with AcOEt (2×400 mL). The combined organic layer was dried over Na₂SO₄, filtered and concentrated to give a dark solid. The crude solid was purified by two consecutive chromatographies on silica gel using a 5% to 75% gradient of eluent B (AcOEt/MeOH:9/1+1% NH4OH) in eluent A (Heptane/CH2Cl2:1/1), leading to the title compound as a brown solid, 15.5 g (51%). HPLC/MS (method A) t_{R} 0.78 minute, M+H 263-265.

### Step C.1: N*2*-(6-Chloro-pyridin-3-yl)-4-fluoro-benzene-1,2-diamine

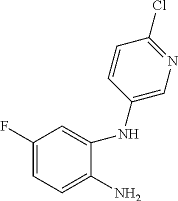 [SMILES: Nc1ccc(F)cc1Nc1ccc(Cl)nc1]

To a suspension of (6-Chloro-pyridin-3-yl)-(5-fluoro-2-nitro-phenyl)-amine (step C.2, 35.5 g, 133 mmol) in EtOH (442 ml) was added tin(II) chloride dihydrate (90 g, 398 mmol) and the mixture was heated to 70° C. for 4 hours. The reaction mixture was cooled down and concentrated to dryness to give a dark residue, which was triturated in the presence of NaOH 2N (199 ml, 398 mmol) and AcOEt (200 mL) for 30 minutes. The tin salts were removed by filtration, the phases of the filtrate separated and the aqueous portion extracted two times with EtOAc (100 ml). The combined organic phases were washed with saturated aqueous sodium bicarbonate solution (2×100 ml) then dried over Na2SO4 and concentrated to a brown solid, 27.7 g (88%). The title product, although shown to be slightly impure by proton NMR spectroscopy, was used as is in the next step. HPLC/MS (method A) t_{R} 1.13 minute, M+H 238-240 (98%).

### Step C.2: (6-Chloro-pyridin-3-yl)-(5-fluoro-2-nitro-phenyl)-amine

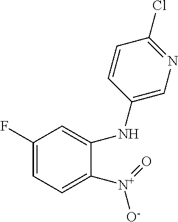 [SMILES: O=[N+]([O-])c1ccc(F)cc1Nc1ccc(Cl)nc1]

Sodium hyride (60% in mineral oil, 7.78 g, 194 mmol) was placed in a 500 mL flask under an inert argon atmosphere. THF (53 ml) was added, followed by a solution of 2-Chloro-5-aminopyridine (5 g, 38.9 mmol) in THF (33 ml), causing a clear red suspension. The reaction mixture was stirred for 30 minutes at rt before the slow addition of a solution of 2,4-difluoro-1-nitro-benzene (8.53 ml, 78 mmol) in THF (65 ml). The resulting red medium was stirred for 30 minutes at RT. The mixture was cooled down to 0° C. and was quenched with tert-butanol (18.60 ml, 194 mmol) in THF (50 mL) before it was allowed to warm up to rt and was stirred vigorously for 40 minutes. The mixture was then concentrated to dryness to give a brown suspension which was treated with 1N HCl (100 mL) and AcOEt (500 mL), causing the appearance of a thick precipitate. The precipitate was filtered ad washed with ether (3×100 mL) to afford the title compound as a brown solid, 7.3 g (70%). HPLC/MS (method A) t_{R} 1.45 minute, M+H 268-270.

### Intermediate D: 1-(4-Bromo-2-methyl-phenyl)-1H-benzoimidazol-2-ylamine

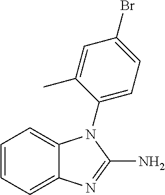 [SMILES: Cc1cc(Br)ccc1-n1c(N)nc2ccccc21]

A mixture of N-(4-Bromo-2-methyl-phenyl)-benzene-1,2-diamine (Step D.1, 686 mg, 2.48 mmol) and cyanogen bromide (535 mg, 4.95 mmol) in acetonitrile (12.4 mL) and water (0.85 mL) was stirred at rt for 22 hours. The reaction medium was concentrated and taken up in CH₂Cl₂ (10 mL) and NaOH 1N (10 mL) using sonication to ensure maximum dissolution of the solids. The phases were separated and the aqueous layer was further extracted with CH₂Cl₂ (2×10 mL). The combined organic phases were then dried over Na2SO4, filtered and evaporated to dryness to afford the title compound as a yellow solid, 734 mg (98%). HPLC/MS (Method A) tᵣ 1.11 minute, M+H 301-303. 1H NMR (Dimethylsulfoxide-d₆) Ppm 1.97 (s, 3H) 6.33 (bs, 2H) 6.58 (d, 1H) 6.84 (t, 1H) 7.00 (t, 1H) 7.22 (d, 1H) 7.28 (d, 1H) 7.60 (d, 1H) 7.75 (s, 1H)

### Step D.1: N-(4-Bromo-2-methyl-phenyl)benzene-1,2-diamine

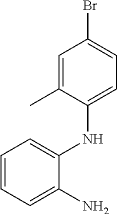 [SMILES: Cc1cc(Br)ccc1Nc1ccccc1N]

A mixture of (4-Bromo-2-methyl-phenyl)-(2-nitro-phenyl)-amine (Step D.2, 850 mg, 2.77 mmol) and tin II chloride, dihydrate (3190 mg, 13.8 mmol) in ethanol (28 mL) was stirred at 70° C. for 16 hours. The reaction mixture was then cooled and concentrated under reduced pressure to about 10 mL. It was then carefully quenched with saturated aqueous NaHCO3 solution (50 mL). The resulting suspension was then treated with EtOAc (50 mL) and filtered through diatomaceous earth. The organic phase was separated and the aqueous portion further extracted with EtOAc (2×25 mL). The combined organic phases were extracted with saturated aqueous NaHCO3 solution (2×50 mL), washed with brine (50 mL), dried over Na2SO4, filtered and evaporated to dryness to afford the title compound as a grey solid, 686 mg (89%). No further purification was performed. HPLC/MS (Method A) 0.02 minute, M+H 276-278. ¹H NMR (Dimethylsulfoxide-d₆) Ppm 2.22 (s, 3H) 4.74 (s, 2H) 6.29 (d, 1H) 6.50-6.57 (m, 2H) 6.74 (d, 1H) 6.80-6.95 (m, 2H) 7.09 (d, 1H) 7.23 (s, 1H)

### Step D.2: (4-Bromo-2-methyl-phenyl)-(2-nitro-phenyl)amine

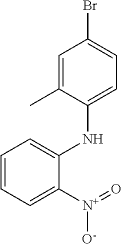 [SMILES: Cc1cc(Br)ccc1Nc1ccccc1[N+](=O)[O-]]

2-Fluoro-1-nitro-benzene (500 μL, 4.73 mmol), 4-Bromo-2-Methylaniline (898 mg, 4.73 mmol), and triethylamine (662 μL, 4.73 mmol) were mixed in a sealed tube and stirred at 180° C. for 18 hours. The crude was partitioned between HCl 1N (50 mL) and EtOAc (50 mL). The phases were separated and the aqueous phase was further extracted with EtOAc (2×50 mL). The combined organic layers were then extracted with HCl 1N (2×50 mL), NaOH 0.1 M (50 mL) and brine (50 mL). The organic phase was then dried over Na2SO4, filtered and evaporated to dryness. The crude was purified by chromatography on silica gel using a 0% to 50% gradient of CH2Cl2 in heptane over ten column volumes followed by an isocratic plateau of 50% CH2Cl2 in heptane over five column volumes, affording the title compound as an orange solid, 443 mg (30%). HPLC/MS (method C) tᵣ 1.02 minute; ¹H NMR (Dimethylsulfoxide-d6) Ppm 2.18 (s, 3H) 6.69 (d, 1H) 6.84 (t, 1H) 7.24 (d, 1H) 7.46 (t, 2H) 7.61 (s, 1H) 8.12 (d, 1H) 9.24 (s, 1H).

### Intermediate E: 1-(4-Bromo-3-methyl-phenyl)-1H-benzoimidazol-2-ylamine

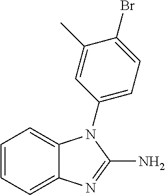 [SMILES: Cc1cc(-n2c(N)nc3ccccc32)ccc1Br]

A mixture of N-(4-Bromo-3-methyl-phenyl)-benzene-1,2-diamine (Step E.1, 723 mg, 2.61 mmol) and cyanogen bromide (564 mg, 5.22 mmol) in acetonitrile (12.4 mL) and water (0.85 mL) was stirred at rt for 22 hours. The reaction medium was concentrated and taken up in CH2Cl2 (10 mL) and NaOH 1N (10 mL) using sonication to ensure maximum dissolution of the solids. The phases were separated and the aqueous layer was further extracted with CH2Cl2 (2×10 mL). The combined organic phases were then dried over Na2SO4, filtered and evaporated to dryness to afford a crude solid. The solid was triturated in tert-butyl methyl ether (10 ml), filtered and washed with heptane to afford the title compound as a pink solid, 287 mg. The filtrate was evaporated to dryness and purified by chromatography on silica gel using a 20% to 100% gradient of eluent B (EtOAc+1% NH4OH) in eluent A (heptane/CH2Cl2 1/1) over fifteen column volumes followed by an isocratic plateau of 100% eluent B over five column volumes, affording another batch of the title compound as a solid, 168 mg, for a combined yield of 455 mg (57%). HPLC/MS (Method A) tᵣ 1.16 minute, M+H 302-304; 1H NMR (Dimethylsulfoxide-d6) Ppm 2.43 (s, 3H) 6.30 (bs, 2H) 6.88 (d, 2H) 7.01 (m, 1H) 7.21 (d, 1H) 7.26 (dd, 1H) 7.50 (s, 1H) 7.80 (d, 1H)

### Step E.1: N-(4-Bromo-3-methyl-phenyl)benzene-1,2-diamine

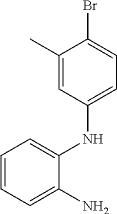 [SMILES: Cc1cc(Nc2ccccc2N)ccc1Br]

A mixture of (4-Bromo-3-methyl-phenyl)-(2-nitro-phenyl)-amine (Step E.2, 973 mg, 3.17 mmol) and tin II chloride, dihydrate (3650 mg, 15.8 mmol) in ethanol (32 mL) was stirred at 70° C. for 16 hours. The reaction mixture was then cooled and concentrated under reduced pressure to about 10 mL. It was then carefully quenched with saturated aqueous NaHCO3 solution (50 mL). The resulting suspension was then treated with EtOAc (50 mL) and filtered through diatomaceous earth. The organic phase was separated and the aqueous portion further extracted with EtOAc (2×25 mL). The combined organic phases were extracted with saturated aqueous NaHCO3 solution (2×50 mL), washed with brine (50 mL), dried over Na2SO4, filtered and evaporated to dryness to afford the title compound as a grey solid, 686 mg (89%). No further purification was performed. HPLC/MS (Method A) tᵣ 1.68 minute, M+H 277-279. 1H NMR (Dimethylsulfoxide-d6) Ppm 2.21 (s, 3H) 4.74 (s, 2H) 6.45 (d, 1H) 6.55 (t, 1H) 6.64 (s, 1H) 6.75 (d, 1H) 6.85 (t, 1H) 6.96 (d, 1H) 7.24 (t, 1H)

### Step E.2: (4-Bromo-3-methyl-phenyl)-(2-nitro-phenyl)-amine

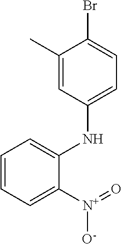 [SMILES: Cc1cc(Nc2ccccc2[N+](=O)[O-])ccc1Br]

2-Fluoro-1-nitro-benzene (500 μL, 4.73 mmol), 4-Bromo-3-Methylaniline (898 mg, 4.73 mmol), and triethylamine (662 μL, 4.73 mmol) were mixed in a sealed tube and stirred at 180° C. for 2 hours under microwave irradiation. The resulting solid was sonicated in the presence of HCl 1M (15 mL) and water (15 mL). The resulting suspension was filtered and washed with water to yield the title compound as an orange solid, 973 mg (67%). HPLC/MS (Method A) tᵣ 2.00 minute, M+H 307-309. ¹H NMR (Dimethylsulfoxide-d6) Ppm 2.34 (s, 3H) 6.92 (t, 1H) 7.10 (dd, 1H) 7.24 (d, 1H) 7.33 (s, 1H) 7.50-7.60 (m, 2H) 8.11 (d, 1H) 9.27 (s, 1H)

### Intermediate F: 1-(5-Bromo-pyridin-2-yl)-1H-benzoimidazol-2-ylamine

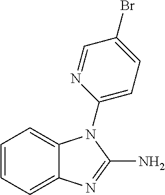 [SMILES: Nc1nc2ccccc2n1-c1ccc(Br)cn1]

A mixture of N-(5-Bromo-pyridin-2-yl)-benzene-1,2-diamine (Step F.1, 3.0 g, 11.4 mmol) and cyanogen bromide (2.4 g, 22.8 mmol) in acetonitrile (28.4 mL) and water (1.9 mL) was stirred at rt for 16 hours. The reaction medium was concentrated and taken up in AcOEt (200 mL) and NaOH 1N (100 mL). The phases were separated and the aqueous layer was further extracted with AcOEt (2×200 mL). The combined organic phases were then dried over Na2SO4, filtered and evaporated to dryness to afford the title compound as a solid, 2.9 g (88%), which was not purified further. HPLC/MS (method A) tᵣ 0.88 minute, M+H 289-291 (95%).

### Step F.1: N-(5-Bromo-pyridin-2-yl)benzene-1,2-diamine

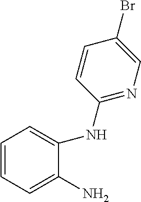 [SMILES: Nc1ccccc1Nc1ccc(Br)cn1]

A mixture of (5-Bromo-pyridin-2-yl)-(2-nitro-phenyl)-amine (Step F.2, 3.9 g, 13.0 mmol) and tin II chloride, dihydrate (14.1 g, 61.2 mmol) in ethanol (41 mL) was stirred at 70° C. for 2 hours. The reaction mixture was then cooled and concentrated under reduced pressure. The resulting crude was then treated with aqueous 1N sodium hydroxide solution (50 mL) and AcOEt (100 mL) under vigorous stirring. The tin salt precipitate was filtered off through diatomaceous earth. The organic phase was separated and the aqueous portion further extracted with EtOAc (2×100 mL). The combined organic phases were dried over Na2SO4, filtered and evaporated to dryness to afford the title compound as a grey solid, 3.0 g (87%). No further purification was performed. HPLC/MS (Method A) tᵣ 1.00 minute, M+H 264-266.

### Step F.2: (5-Bromo-pyridin-2-yl)-(2-nitro-phenyl)amine

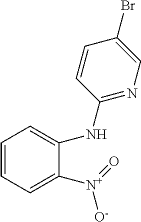 [SMILES: O=[N+]([O-])c1ccccc1Nc1ccc(Br)cn1]

A mixture of 5-bromo-2-iodopyridine (5.0 g, 17.3 mmol), 2-nitroaniline (2.4 g, 17.2 mmol), cesium carbonate (28.1 g, 86.2 mmol), palladium diacetate (116 mg, 0.51 mmol), triethylamine (2.40 mL, 17.2 mmol) and rac-BINAP (332 mg, 0.51 mmol) in toluene (58 mL) under argon was heated to reflux for 24 hours. The reaction mixture was then diluted in 100 mL of AcOEt, filtered over a pad of celite and concentrated under reduced pressure to give a dark red solid. The red solid was taken up in EtOAc (400 mL) and water (200 mL). The phases were separated, the aqueous portion further extracted with EtOAc (2×200 mL), the combined organic layers dried over sodium sulfate and concentrated. The crude product was purified by chromatography on silica gel, using a 10% to 100% gradient of eluent B (MTBE+5% 7N NH₃ in MeOH) in eluent A (Heptane), leading to the title compound as an orange solid, 3.9 g (77%). HPLC/MS (method A) t_{R} 1.72 minute, M+H 294-296.

### Intermediate G: 1-(4-Bromo-phenyl)-7-methyl-1H-benzoimidazol-2-ylamine

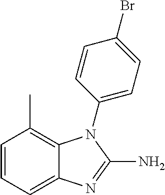 [SMILES: Cc1cccc2nc(N)n(-c3ccc(Br)cc3)c12]

The title compound I-(4-Bromo-phenyl)-7-methyl-1H-benzoimidazol-2-ylamine was synthesized in three steps in a manner analogous to that used for the synthesis of intermediate B, using 1-Bromo-4-iodobenzene instead of 2-chloro-5-iodopyridine and 2-methyl-6-nitroaniline instead of 2-nitroaniline in the first synthetic step. HPLC/MS (method A) t_{R} 1.72 minute, M+H 302-304, 1H NMR (Dimethylsulfoxide-d6) Ppm 1.78 (s, 3H) 6.04 (br. s., 2H) 6.61 (d, 1H) 6.88 (t, 1H) 7.06 (d, 1H) 7.42 (d, 2H) 7.75 (d, 2H)

### Intermediate H: 1-(4-Bromo-phenyl)-6-fluoro-1H-benzoimidazol-2-ylamine

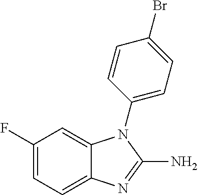 [SMILES: Nc1nc2ccc(F)cc2n1-c1ccc(Br)cc1]

The title compound 1-(4-Bromo-phenyl)-6-fluoro-1H-benzoimidazol-2-ylamine was synthesized in three steps in a manner analogous to that used for the synthesis of intermediate A, using 2,4-difluoro-1-nitro-benzene instead of 2-fluoro-1-nitro-benzene in the first synthetic step. HPLC/MS (method A) t_{R} 0.96 minute, M+H 306-308.

### Intermediate I: 1-(4-Bromo-phenyl)-5,6-difluoro-1H-benzoimidazol-2-ylamine

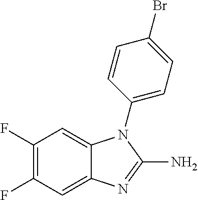 [SMILES: Nc1nc2cc(F)c(F)cc2n1-c1ccc(Br)cc1]

The title compound 1-(4-Bromo-phenyl)-5,6-fluoro-1H-benzoimidazol-2-ylamine was synthesized in three steps in a manner analogous to that used for the synthesis of intermediate A, using 2,4,5-trifluoro-1-nitro-benzene instead of 2-fluoro-1-nitro-benzene in the first synthetic step. HPLC/MS (method A) t_{R} 1.30 minute, M+H 324-326.

### Intermediate J: 1-(6-Chloro-pyridazin-3-yl)-7-methyl-1H-benzoimidazol-2-ylamine

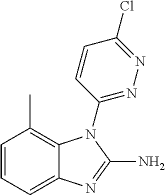 [SMILES: Cc1cccc2nc(N)n(-c3ccc(Cl)nn3)c12]

The title compound was synthesized in a manner analogous to that used for the synthesis of intermediate A using N*2*-(6-Chloro-pyridazin-3-yl)-3-methyl-benzene-1,2-diamine (Step J.1) instead of N-(4-Bromo-phenyl)-benzene-1,2-diamine (Step A.1). HPLC/MS (method A) t_{R} 0.79 minute, M+H 260 (90%), ¹H NMR (Dimethylsulfoxide-d6) Ppm 1.77 (s, 3H) 6.39 (s, 2H) 6.68 (d, 1H) 6.96 (t, 1H) 7.11 (d, 1H) 8.11 (d, 1H) 8.22 (d, 1H).

### Step J.1: N*2*-(6-Chloro-pyridazin-3-yl)-3-methyl-benzene-1,2-diamine

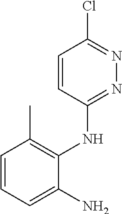 [SMILES: Cc1cccc(N)c1Nc1ccc(Cl)nn1]

The title compound was synthesized in a manner analogous to that used for the synthesis of N-(4-Bromo-phenyl)-benzene-1,2-diamine (Step A.1) using (6-Chloro-pyridazin-3-yl)-(2-methyl-6-nitro-phenyl)-amine (Step J.2) instead of (4-Bromo-phenyl)-(2-nitrophenyl)-amine (Step A.2). HPLC/MS (method A) t_{R} 0.91 minute, M+H 235.

### Step J.2: (6-Chloro-pyridazin-3-yl)-(2-methyl-6-nitro-phenyl)-amine

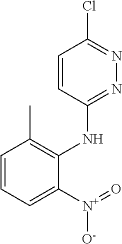 [SMILES: Cc1cccc([N+](=O)[O-])c1Nc1ccc(Cl)nn1]

Sodium hydride (60% oil dispersion, 593 mg, 14.83 mmol) was suspended in DMF (16.1 mL). 3-amino-6-chloropyridazine (1921 mg, 14.83 mmol) was added in small portions, causing a yellow coloration and a strong gaseous evolution. The resulting slurry was stirred for 10 minutes at rt and 2-fluoro-3-nitrotoluene (0.785 mL, 6.45 mmol) was added as a neat liquid, causing a dramatic shift to a deep red colour. The resulting reaction mixture was stirred at rt for 50 minutes. The medium was carefully quenched in 30 mL MeOH and 10 mL water. The medium was evaporated to a thick oil. The crude was taken up in EtOAc (50 mL) and sonicated for few minutes. The solid materials were removed by filtration and the filtrate was washed successively with saturated aqueous sodium bicarbonate solution (2×50 mL), 1M aqueous HCl (50 mL) and brine (50 mL). The combined organics were dried over Na2SO4 and concentrated to yield the title compound as a crystalline, yellow solid, m=1.63 g (96%). HPLC/MS (method A) t_{R} 1.20 minute, M+H 265.

### Intermediate K: 1-(6-Chloro-pyridazin-3-yl)-1H-benzoimidazol-2-ylamine

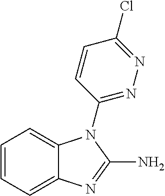 [SMILES: Nc1nc2ccccc2n1-c1ccc(Cl)nn1]

To 2-aminobenzoimidazole (2 g, 15.02 mmol) in DMF (17.67 ml) was added NaH (60% in mineral oil, 0.360 g, 15.02 mmol). The reaction mixture was stirred at rt for an hour. A solution of 3,6-dichloropyridazine (2.238 g, 15.02 mmol) in DMF (12.37 ml) was added dropwise, and the reaction mixture was stirred for 20 hours at RT. The reaction mixture was then treated with water (100 mL) and AcOEt (20 mL). The phases were separated and the aqueous layer was extracted further with AcOEt (2×10 mL). The combined organic phases were dried over Na2SO4, filtered and concentrated under reduced pressure to give a brown oil. The crude was diluted in CH2Cl2 (10 mL) and MeOH (5 mL) causing the occurrence a yellow precipitate. The precipitate was filtered and dried under reduced pressure to give the title compound as a yellow solid 1.0 g (27%). HPLC/MS (method A) t_{R} 0.68 minute, M+H 246, ¹H NMR (Dimethylsulfoxide-d6) Ppm 6.80 (br. s., 2H) 6.96 (t, 1H) 7.11 (t, 1H) 7.28 (t, 2H) 8.10-8.26 (m, 2H).

### Intermediate L: N-[4′-(2-Amino-benzoimidazol-1-yl)-biphenyl-2-yl]-acetamide

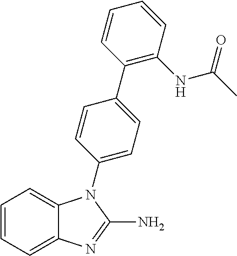 [SMILES: CC(=O)Nc1ccccc1-c1ccc(-n2c(N)nc3ccccc32)cc1]

Intermediate A (355 mg, 1.23 mmol), 2-acetamidophenylboronic acid (249 mg, 1.35 mmol) and Pd(Ph3P)4 (72 mg, 61 μmol) were suspended in DME (6.2 mL) and 2M aqueous Na2CO3 solution (3.1 mL, 6.1 mmol). The resulting reaction mixture was stirred for 17 minutes at 150° C. under microwave irradiation. The reaction mixture was then diluted with MeOH (6 mL) and filtered through a pad of diatomaceous earth which was rinsed with EtOAc (2×10 mL). The resulting organic filtrate was evaporated to dryness, the residue was taken up in EtOAc (30 mL) and water (30 mL), causing a precipitation to occur. The solids were filtered off and dried under vacuum to furnish the title compound, 280 mg (63%). HPLC/MS (method A) t_{R} 0.91 minute, M+H 343.0. ¹H NMR (Dimethylsulfoxide-d6) Ppm 1.97 (s, 3H) 6.27 (bs, 2H) 6.90-7.70 (m, 12H) 9.26 (s, 1H).

### Intermediate M: 1-(4-Bromo-phenyl)-3-(2-chloro-benzyl)-1,3-dihydro-benzoimidazol-2-ylideneamine

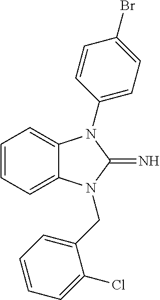 [SMILES: N=c1n(Cc2ccccc2Cl)c2ccccc2n1-c1ccc(Br)cc1]

To a suspension of intermediate A (6.26 g, 21.6 mmol) in MeCN (216 mL) were added potassium iodide (3.66 g, 21.6 mmol) and 2-chlorobenzylbromide (2.82 mL, 21.6 mmol). The resulting slurry was heated to 110° C. for 10 min under microwave irradiation. The medium was evaporated to dryness. The crude was taken up in water (250 mL) and CH2Cl2 (250 mL). The mixture was shaken and the phases were separated. The aqueous phase was extracted with CH2Cl2 (2×100 mL) and the organics were dried over Na2SO4 and concentrated to a crude solid. The crude product was purified by chromatography on silica gel, using a 10% to 100% gradient of eluent B (EtOAc+1% NH4OH) in eluent A (CH2Cl2/heptane:1/1), leading to the title compound as an dark brown solid, 4.43 g (50%). HPLC/MS (method A) t_{R} 1.24 minute, M+H 412-414. ¹H NMR (Dimethylsulfoxide-d6) Ppm 5.21 (s, 2H) 6.90 (m, 4H) 7.08 (d, 1H) 7.31 (m, 2H) 7.56 (m, 3H) 7.82 (d, 2H).

### Intermediate N: 4-Bromomethyl-indole-1-carboxylic acid tert-butyl ester

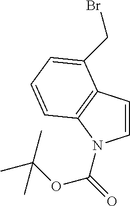 [SMILES: CC(C)(C)OC(=O)n1ccc2c(CBr)cccc21]

To a solution of 4-Hydroxymethyl-indole-1-carboxylic acid tert-butyl ester (step N.1, 17.0 g, 68.7 mmol) in CH2Cl2 (229 ml) was added tetrabromomethane (25.1 g, 76 mmol) and the yellow clear solution cooled to 0° C. Then triphenylphosphine (27.0 g, 103 mmol) was added in portions over the course of 10 minutes. The resulting mixture was stirred at 0° C. for 1 hr. The mixture was then treated with water (150 mL) and the medium was vigorously stirred for an hour at rt. The two phases were separated, the aqueous layer was extracted with CH2Cl2 (250 mL), the combined organic layers were dried over Na2SO4, filtered and concentrated to a clear brown oil. The crude product was purified by chromatography on silica gel, eluting with CH2Cl2/heptane:1/1, leading to the title compound as a clear oil, 16.2 g (76%). HPLC/MS (method A) t_{R} 1.88 minute, M-Br 230. 1H NMR (Dimethylsulfoxide-d₆) Ppm 1.64 (s, 9H) 5.00 (s, 2H) 6.92 (d, 1H) 7.25-7.38 (m, 2H) 7.77 (d, 1H) 8.04 (d, 1H).

### Step N.1: 4-Hydroxymethyl-indole-1-carboxylic acid tert-butyl ester

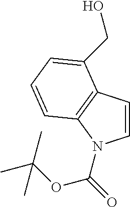 [SMILES: CC(C)(C)OC(=O)n1ccc2c(CO)cccc21]

To a solution of indole-1,4-dicarboxylic acid 1-tert-butyl ester 4-methyl ester (step N.2, 20.0 g, 72.6 mmol) in THF (363 ml) at −78° C. under Ar was added DIBAL-H 1M in cyclohexane (145.0 ml, 145 mmol) dropwise. The resulting solution was stirred at −78° C. for 20 minutes, allowed to reach rt slowly and then stirred at rt temperature for 16 hours. The reaction mixture was then cooled to 0° C. and Rochelle's salt solution (200 mL) was carefully added (strong exotherm). The resulting mixture was stirred for 2 hours. The phases were separated and the organic layer was concentrated to remove the maximum of the solvent. The resulting solution was diluted with AcOEt (400 mL) and the organic phase was then washed with Rochelle's salt solution (2*200 mL), water (1×200 mL) and brine (1×200 mL). The organic layer was then dried over Na2SO4, filtered and evaporated to dryness to give a clear brown oil, 18.1 g (96%). HPLC/MS (method A) t_{R} 1.38 minute. ¹H NMR (Dimethylsulfoxide-d6) Ppm 1.63 (s, 9H) 4.75 (d, 2H) 5.24 (t, 1H) 6.81 (d, 1H) 7.18-7.36 (m, 2H) 7.66 (d, 1H) 7.96 (d, 1H).

### Step N.2: Indole-1,4-dicarboxylic acid 1-tert-butyl ester 4-methyl ester

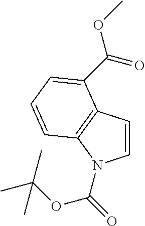 [SMILES: COC(=O)c1cccc2c1ccn2C(=O)OC(C)(C)C]

To a solution of methyl Indole-4-carboxylate (5 g, 28.5 mmol) in MeCN (40.8 ml) was added DMAP (0.174 g, 1.427 mmol) followed by Boc2O (7.95 ml, 34.2 mmol). The resulting solution was stirred for 16 hours. The reaction mixture was then evaporated to dryness. The crude was diluted in EtOAc (200 mL) and washed successively with 10% aqueous citric acid solution (3×100 mL), saturated aqueous NaHCO3 solution (2×100 mL), and saturated aqueous NaCl solution (1×100 mL). The organic layer was dried over Na2SO4, filtered and concentrated to give the title compound as a clear oil, 7.86 g (100%), no further purification carried out. HPLC/MS (method A) t_{R} 1.41 minute, M+H 276. 1H NMR (Dimethylsulfoxide-d6) Ppm 1.64 (s, 9H) 3.92 (s, 3H) 7.20 (d, 1H) 7.44 (t, 1H) 7.84 (d, 1H) 7.90 (dd, 1H) 8.36 (d, 1H).

### Intermediate O: 4-[3-(4-Bromo-phenyl)-2-imino-2,3-dihydro-benzoimidazol-1-ylmethyl]-indole-1-carboxylic acid tert-butyl ester

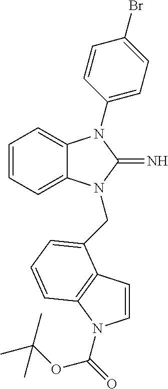 [SMILES: CC(C)(C)OC(=O)n1ccc2c(Cn3c(=N)n(-c4ccc(Br)cc4)c4ccccc43)cccc21]

To a suspension of intermediate A (500 mg, 1.73 mmol) in MeCN (17 mL) were added potassium iodide (288 mg, 1.73 mmol) and intermediate N (538 mg, 1.73 mmol). The resulting slurry was heated to 110° C. for 30 min. The medium was filtered, the cake washed with EtOAc (2×10 mL) and the filtrate evaporated to dryness. The crude product was purified by chromatography on silica gel, using a 15% to 70% gradient of eluent B (EtOAc+1% NH4OH) in eluent A (heptane), leading to the title compound as a brown foam, 615 mg (68%). HPLC/MS (method A) t_{R} 1.46 minute, M+H 517-519. ¹H NMR (Dimethylsulfoxide-d6) Ppm 1.63 (s, 9H) 5.44 (s, 2H) 6.77-6.85 (m, 1H) 6.85-6.95 (m, 3H) 7.07 (d, 1H) 7.21 (d, 1H) 7.30 (t, 1H) 7.56 (d, 2H) 7.73 (d, 1H) 7.82 (d, 2H) 8.01 (d, 1H).

### Intermediate P: [1-(4-Bromo-phenyl)-3-(2-chloro-benzyl)-1,3-dihydro-benzoimidazol-(2E)-ylidene]-carbamic acid tert-butyl ester

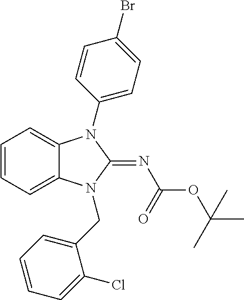 [SMILES: CC(C)(C)OC(=O)/N=c1\n(Cc2ccccc2Cl)c2ccccc2n1-c1ccc(Br)cc1]

To a solution of intermediate M (1000 mg, 2.42 mmol) in DCE (12 ml) were added DIPEA (0.51 mL, 2.91 mmol) and Boc2O (686 mg, 3.15 mmol) and the resulting mixture stirred at 70° C. for 44 hours. The medium was then diluted with CH2Cl2 (50 mL) and washed with water (50 mL). The organic phase was then dried over Na2SO4 and concentrated to an oil which was purified by chromatography on silica gel, eluting with a 90/10 mixture of heptane and EtOAc. The title compound was isolated as a solid, 1.0 g (81%%). HPLC/MS (method A) t_{R} 1.87 minute, M+H 511.9. ¹H NMR (Dimethylsulfoxide-d6) Ppm 1.24 (s, 9H) 5.46 (s, 2H) 7.03 (m, 2H) 7.10-7.30 (m, 5H) 7.43 (m, 3H) 7.71 (d, 2H).

### Intermediate Q: [1-(2′-Amino-biphenyl-4-yl)-3-(2-chloro-benzyl)-1,3-dihydrobenzoimidazol-(2E)-ylidene]-carbamic acid tert-butyl ester

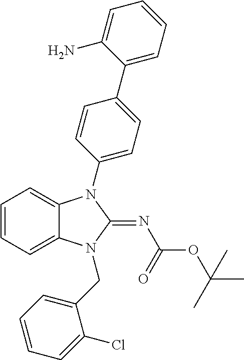 [SMILES: CC(C)(C)OC(=O)/N=c1\n(Cc2ccccc2Cl)c2ccccc2n1-c1ccc(-c2ccccc2N)cc1]

Intermediate P (1008 mg, 1.96 mmol), 2-aminophenylboronic acid pinacol ester (481 mg, 2.15 mmol) and Pd(Ph3P)₄ (115 mg, 98 μmol) were suspended in DME (10 mL) and 2M aqueous Na2CO3 solution (4.9 mL, 9.8 mmol). The resulting reaction mixture was stirred for 150 minutes at 90° C. The reaction mixture was then diluted with MeOH (20 mL) and filtered through a pad of diatomaceous earth which was rinsed with EtOAc (2×50 mL). The resulting organic filtrate was evaporated to dryness, the residue was taken up in EtOAc (50 mL) and water (50 mL). The phases were separated and the organic portion was washed with 10% aqueous sodium carbonate solution (50 mL), brine (50 mL), dried over Na2SO4, and concentrated under reduced pressure to yield the title compound as a brown residue which was not purified further. HPLC/MS (method A) t_{R} 1.80 minute, M+H 525.0 (M+2H)/2 263.0. ¹H NMR (Dimethylsulfoxide-d6) Ppm 1.08 (s, 9H) 4.85 (s, 2H) 5.38 (s, 2H) 6.67 (t, 1H) 6.79 (d, 1H) 6.95-7.10 (m, 3H) 7.15-7.25 (m, 4H) 7.30-7.40 (m, 2H) 7.59 (m, 5H).

### Intermediate R: rac-N-[2-Bromo-4-(1-hydroxy-ethyl)-phenyl]-acetamide

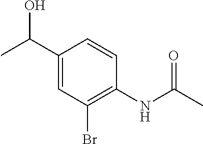

3-Bromo-4-acetamidobenzophenone (500 mg, 1.952 mmol) was suspended in MeOH (3905 μl) and treated with NaBH4 (148 mg, 3.90 mmol). The resulting reaction mixture was stirred at rt for 25 minutes before evaporation to dryness. The residue was taken up in EtOAc (10 mL) and washed successively with 1M aqueous sodium hydroxide solution (3×10 mL) and brine (10 mL). The organic phase was then dried over Na2SO4, filtered and evaporated to dryness to give the title compound as a red oil, 300 mg (59%), which was used without further purification. HPLC/MS (method A) t_{R} 0.83 minute, M+H 257.9. 1H NMR (Dimethylsulfoxide-d6) Ppm 1.30 (d, 3H) 2.06 (s, 3H) 4.70 (m, 1H) 5.28 (d, 1H) 7.29 (d, 1H) 7.47 (d, 1H) 7.59 (s, 1H) 9.44 (s, 1H)

### Intermediate S: 4-[3-(2-Chloro-benzyl)-2-imino-2,3-dihydro-benzoimidazol-1-yl]-phenylboronic acid

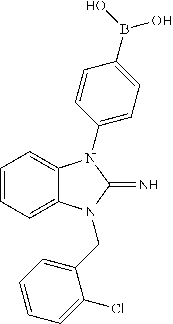 [SMILES: N=c1n(Cc2ccccc2Cl)c2ccccc2n1-c1ccc(B(O)O)cc1]

A mixture of intermediate M (150 mg, 0.363 mmol), bis(pinacolato)diboron (185 mg, 0.727 mmol), PdCl2(dppf). CH2Cl2 adduct (29.7 mg, 0.036 mmol) and potassium acetate (107 mg, 1.090 mmol) in DMF under Ar was stirred for one hour at 80° C. The reaction mixture was then cooled and evaporated to dryness. The residue was triturated in EtOAc (10 mL) and the resulting brown suspension was filtered through a pad of celite which was washed with EtOAc (2×5 mL). The filtrate was then concentrated to a crude oil which was purified by reverse-phase preparative HPLC (Method E, gradient from 20% ((MeCN/MeOH 1/3)+0.1% TFA) in (Water+0.1% TFA) to 80% over 14 minutes). Product-containing fractions were pooled and lyophilized to yield the title compound as its TFA salt, 113 mg (63%). HPLC/MS (method A) t_{R} 1.14 minute, M+H 378.1.

### Intermediate T: 4-{3-(4-Bromo-phenyl)-2-[(E)-tert-butoxycarbonylimino]-2,3-dihydrobenzoimidazol-1-ylmethyl}-indole-1-carboxylic acid tert-butyl ester

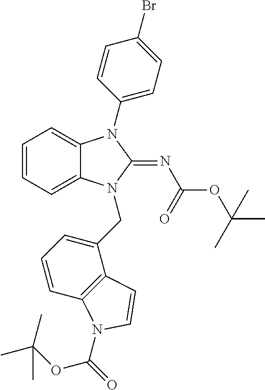 [SMILES: CC(C)(C)OC(=O)/N=c1\n(Cc2cccc3c2ccn3C(=O)OC(C)(C)C)c2ccccc2n1-c1ccc(Br)cc1]

To a solution of intermediate 0 (500 mg, 0.966 mmol) in DCE (4.80 mL) were added DIPEA (0.20 mL, 1.063 mmol) and Boc2O (232 mg, 1.063 mmol). The resulting solution was stirred at 70° C. for 40 hours. The medium was then diluted with CH2Cl2 (20 mL) and washed with 10% aqueous citric acid solution (2×20 mL), 1M aqueous sodium hydroxyide solution (20 mL) and brine (20 mL). The organic phase was dried over Na2SO4 and evaporated to a crude oil. The crude product was purified by chromatography on silica gel, eluting with a 9/1 mixture of (heptane/CH₂Cl₂: 1/1)/EtOAc, to furnish the title compound as a solid, 600 mg (quantitative). HPLC/MS (Method C) t_{R} 1.18 minute, M+H 617.0-619.0.

### Intermediate U: 4-Acetylamino-3-(4,4,5,5-tetramethyl-[1,3,2]dioxaborolan-2-yl)-benzoic acid methyl ester

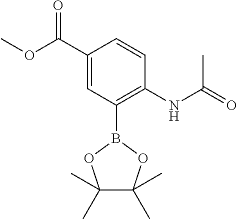 [SMILES: COC(=O)c1ccc(NC(C)=O)c(B2OC(C)(C)C(C)(C)O2)c1]

4-Acetylamino-3-bromo-benzoic acid methyl ester (100 mg, 0.368 mmol), Pd(PPh3)2Cl2 (12.90 mg, 0.018 mmol), bis(pinacolato)diboron (112 mg, 0.441 mmol) and KOAc (108 mg, 1.103 mmol) were placed in a sealed vial under N2. Dioxane (3.7 mL) was added and the resulting reaction mixture was stirred at 80° C. for 1.5 hours. The mixture was then filtered through diatomaceous earth, and evaporated to dryness to furnish the title compound as a black oil which was used without further purification. HPLC/MS (Method A) t_{R} 0.48 minute, fragment ion 220.0 amu in positive ionisation mode (100%).

### Intermediate V: 4-[3-(4-Dihydroxyboronyl-phenyl)-2-imino-2,3-dihydro-benzoimidazol-1-ylmethyl]-indole-1-carboxylic acid tert-butyl ester

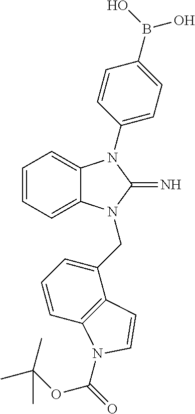 [SMILES: CC(C)(C)OC(=O)n1ccc2c(Cn3c(=N)n(-c4ccc(B(O)O)cc4)c4ccccc43)cccc21]

4-[3-(4-Bromo-phenyl)-2-imino-2,3-dihydro-benzoimidazol-1-ylmethyl]-indole-1-carboxylic acid tert-butyl ester (intermediate O, 135 mg, 0.261 mmol), Pd(dppf)Cl2.CH2Cl2 adduct (21.31 mg, 0.026 mmol), bis(pinacolato)diboron (133 mg, 0.522 mmol) and KOAc (77 mg, 0.783 mmol) were placed in a sealed vial under N2. DMF (1.3 mL) was added and the resulting reaction mixture was stirred at 80° C. for 0.5 hours. The mixture was then evaporated to dryness and purified by reverse-phase preparative HPLC (Method E, Gradient from 40% ((MeCN/MeOH:1/3)+0.1% TFA) in (Water+0.1% TFA) to 70% over 14 minutes) to furnish the title compound, 122 mg (97%). HPLC/MS (Method A) t_{R} 1.29 minute, M+H 483.2.

### Intermediate W: 1-(5-Bromo-pyridin-2-yl)-3-(2-chloro-benzyl)-1,3-dihydro-benzoimidazol-2-ylideneamine

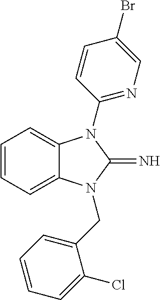 [SMILES: N=c1n(Cc2ccccc2Cl)c2ccccc2n1-c1ccc(Br)cn1]

To a suspension of intermediate F (2.90 g, 10.0 mmol) in MeCN (11.4 mL) were added potassium iodide (1.68 g, 10.0 mmol) and 2-chlorobenzylbromide (1.30 mL, 10.0 mmol). The resulting slurry was heated to 110° C. for 10 min under microwave irradiation. The medium was evaporated to dryness. The crude was taken up in water (100 mL) and CH2Cl2 (200 mL). The mixture was shaken and the phases were separated. The aqueous phase was extracted with CH2Cl2 (2×100 mL) and the organics were dried over Na2SO4 and concentrated to a crude solid. The crude product was purified by chromatography on silica gel, using a 10% to 100% gradient of eluent B (EtOAc+1% (7N NH3 in MeOH)) in eluent A (heptane), leading to the title compound as a yellow, 1.90 g (51%). HPLC/MS (method A) t_{R} 1.20 minute, M+H 412.9-414.9.

### Intermediate X: N-[4-Methyl-2-(4,4,5,5-tetramethyl-[1,3,2]dioxaborolan-2-yl)-phenyl]-acetamide

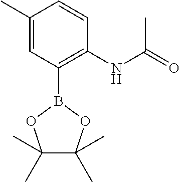 [SMILES: CC(=O)Nc1ccc(C)cc1B1OC(C)(C)C(C)(C)O1]

To a solution of 4-Methyl-2-(4,4,5,5-tetramethyl-[1,3,2]dioxaborolan-2-yl)-phenylamine (2.0 g, 8.58 mmol) in CH2Cl2 (28.6 ml) were added acetyl chloride (1.219 ml, 17.16 mmol) and pyridine (1.388 ml, 17.16 mmol). The reaction mixture was stirred for an hour at rt. The reaction mixture was then diluted with CH2Cl2 (200 mL) and washed with 1N aqueous sodium hydroxide solution (100 mL). The aqueous layer was extracted with CH2Cl2 (2×100 mL). The organic layers were combined and dried over Na2SO4, filtered and concentrated under reduced pressure to give the title product, 1.6 g (68%), which was used without purification. HPLC/MS (method A) t_{R} 0.47 minute, ion detected at 176.0 amu in positive ionisation mode (100%). ¹H NMR (Dimethylsulfoxide-d6) Ppm 1.17 (s, 12H) 2.20-2.31 (m, 6H) 6.91 (d, 1H) 7.04 (dd, 1H) 7.20 (d, 1H) 11.68 (br. s., 1H).

### Intermediate Y: N-[4′-(2-Amino-7-methyl-benzoimidazol-1-yl)-5-methyl-biphenyl-2-yl]-acetamide

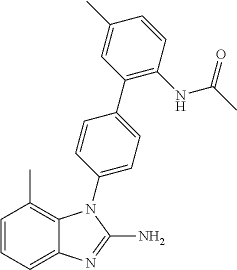 [SMILES: CC(=O)Nc1ccc(C)cc1-c1ccc(-n2c(N)nc3cccc(C)c32)cc1]

A suspension of intermediate G (500 mg, 1.655 mmol), intermediate X (501 mg, 1.820 mmol), and Pd(PPh3)₂Cl2 (58.1 mg, 0.083 mmol) in DME (8.3 mL) and 2M aqueous Na2CO3 solution (4.14 mL, 8.27 mmol) was heated to 150° C. for 16 minutes under microwave irradiation. The reaction mixture was then diluted with MeOH (2 mL) and filtered through a pad of diatomaceous earth which was rinsed with EtOAc (3×10 mL). The resuiting organic filtrate was evaporated to dryness. The residue was purified by chromatography on silica gel, using a 10% to 100% gradient of eluent B (EtOAc+1% NH4OH) in eluent A (heptane), yielding the title product as a grey solid, 522 mg (85%). HPLC/MS (Method A) t_{R} 1.02 minute, M+H 371.0.

### Intermediate Z: 3-(4-Bromo-phenyl)-1-(2-chloro-benzyl)-4-methyl-1,3-dihydrobenzoimidazol-2-ylideneamine

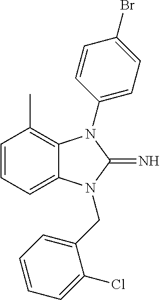 [SMILES: Cc1cccc2c1n(-c1ccc(Br)cc1)c(=N)n2Cc1ccccc1Cl]

The title compound was synthesized in a manner analogous to that used for the synthesis of intermediate W, using intermediate G instead of intermediate F. HPLC/MS (Method A) t_{R} 1.27 minute, M+H 427.

### Intermediate AA: 4-[3-(4-Bromo-phenyl)-2-imino-4-methyl-2,3-dihydro-benzoimidazol-1-ylmethyl]-indole-1-carboxylic acid tert-butyl ester

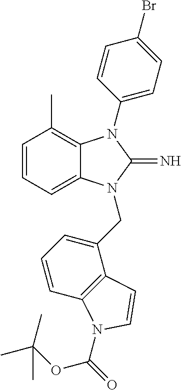 [SMILES: Cc1cccc2c1n(-c1ccc(Br)cc1)c(=N)n2Cc1cccc2c1ccn2C(=O)OC(C)(C)C]

The title compound was synthesized in a manner analogous to that used for the synthesis of intermediate O, using intermediate G instead of intermediate A. HPLC/MS (Method A) t_{R} 1.48 minute, M+H 533.

### Intermediate AB: N-[4′-(2-Amino-5,6-difluoro-benzoimidazol-1-yl)-5-chloro-biphenyl-2-yl]-acetamide

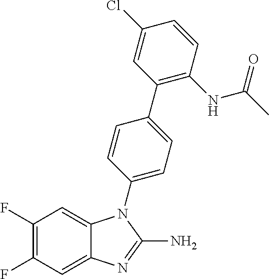 [SMILES: CC(=O)Nc1ccc(Cl)cc1-c1ccc(-n2c(N)nc3cc(F)c(F)cc32)cc1]

The title compound was synthesized in a manner analogous to that used for the synthesis of intermediate Y, using intermediate I instead of intermediate A and 2-Acetamido-5-chlorophenylboronic acid pinacol ester instead of intermediate X. HPLC/MS (Method A) t_{R} 1.86 minute, M+H 413.

### Intermediate AC: 1-(6-Chloro-pyridazin-3-yl)-3-(2-chloro-benzyl)-1,3-dihydrobenzoimidazol-2-ylideneamine

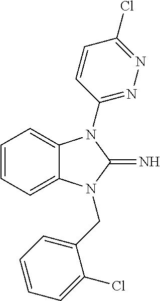 [SMILES: N=c1n(Cc2ccccc2Cl)c2ccccc2n1-c1ccc(Cl)nn1]

The title compound was synthesized in a manner analogous to that used for the synthesis of intermediate W, using intermediate K instead of intermediate F. HPLC/MS (Method A) t_{R} 0.99 minute, M+H 369.9-372.0. ¹H NMR (Dimethylsulfoxide-d₆) Ppm 5.27 (s, 2H) 6.73-7.15 (m, 5H) 7.22-7.41 (m, 2H) 7.55 (dd, 1H) 7.83 (br. s., 1H) 8.09 (d, 1H) 8.86 (br. s., 1H).

### Intermediate AD: 4-[3-(6-Chloro-pyridazin-3-yl)-2-imino-2,3-dihydro-benzoimidazol-1-ylmethyl]-indole-1-carboxylic acid tert-butyl ester

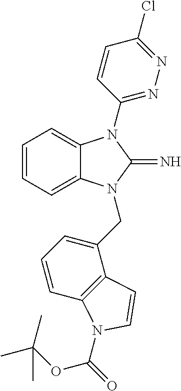 [SMILES: CC(C)(C)OC(=O)n1ccc2c(Cn3c(=N)n(-c4ccc(Cl)nn4)c4ccccc43)cccc21]

The title compound was synthesized in a manner analogous to that used for the synthesis of intermediate O, using intermediate K instead of intermediate A. HPLC/MS (Method A) t_{R} 1.31 minute, M+H 475.0-477.0.

### Intermediate AE: 1-(2-Chloro-benzyl)-3-(6-chloro-pyridazin-3-yl)-4-methyl-1,3-dihydrobenzoimidazol-2-ylideneamine

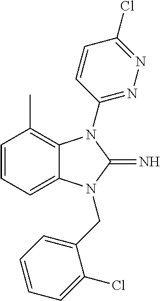 [SMILES: Cc1cccc2c1n(-c1ccc(Cl)nn1)c(=N)n2Cc1ccccc1Cl]

The title compound was synthesized in a manner analogous to that used for the synthesis of intermediate W, using intermediate J instead of intermediate F. HPLC/MS (Method A) t_{R} 1.12 minute, M+H 384.0-386.0

### Intermediate AF: 4-[3-(6-Chloro-pyridazin-3-yl)-2-imino-4-methyl-2,3-dihydrobenzoimidazol-1-ylmethyl]-indole-1-carboxylic acid tert-butyl ester

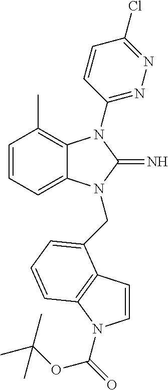 [SMILES: Cc1cccc2c1n(-c1ccc(Cl)nn1)c(=N)n2Cc1cccc2c1ccn2C(=O)OC(C)(C)C]

The title compound was synthesized in a manner analogous to that used for the synthesis of intermediate 0, using intermediate J instead of intermediate A. HPLC/MS (Method A) t_{R} 1.36 minute, M+H 489.0-491.0.

### Intermediate AG: 4-Bromomethyl-benzoimidazole-1-carboxylic acid tert-butyl ester

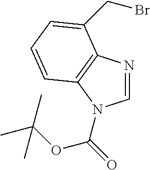 [SMILES: CC(C)(C)OC(=O)n1cnc2c(CBr)cccc21]

To a solution of 4-Hydroxymethyl-benzoimidazole-1-carboxylic acid tert-butyl ester (Step AG1, 330 mg, 1.329 mmol) in Et₂₀ (5 ml) at 0° C. under Ar was added PBr3 (1M in CH₂Cl₂) (1.462 ml, 1.462 mmol), dropwise, causing a thick precipitation. After 5 minutes at 0° C., the medium was allowed to reach rt. After 1 h at rt, PBr3 (1M in CH₂Cl₂) (0.133 ml, 0.133 mmol) was added under stirring, followed by CH2Cl2 (2 ml). After a further 1 h at rt, the medium was carefully quenched with saturated sodium bicarbonate solution (20 ml, strong CO2 evolution) and extracted with EtOAc (3×15 ml). Drying of the combined organics over Na2SO4 and concentration afforded a clear oil. The oil was purified by chromatography on silica gel, using a 0% to 100% gradient of eluent B (EtOAc/CH₂Cl₂/heptane:1/2/2) in eluent A (heptane/CH2Cl2:1/1), yielding the title product as a colorless oil, 153 mg (37%). HPLC/MS (Method A) t_{R} 1.70 minute, M+H 310.9-312.9. 1H NMR (DMSO-d₆) Ppm 1.69 (s, 9H) 4.95 (s, 2H) 7.38 (m, 2H) 7.94 (d, 1H) 8.49 (s, 1H).

### Step AG1: 4-Hydroxymethyl-benzoimidazole-1-carboxylic acid tert-butyl ester

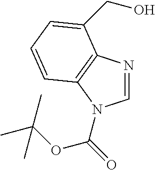 [SMILES: CC(C)(C)OC(=O)n1cnc2c(CO)cccc21]

To a solution of (1H-Benzoimidazol-4-yl)-methanol (Step AG2, 600 mg, 4.05 mmol) in MeCN (32 mL) and Water (8 mL) was added sodium bicarbonate (680 mg, 8.10 mmol) followed by BOC₂O (1.081 mL, 4.66 mmol). The resulting mixture was stirred at rt for 4 hrs before careful evaporation to dryness. The crude was taken up in AcOEt (200 mL) and extracted with 10% aqueous citric acid solution (3×100 mL), water (2×100 mL) and brine (100 mL). The organic layer was dried over Na2SO4, filtered and concentrated to give an oily white solid (855 mg, 85%). No purification was required. HPLC/MS (Method A) t_{R} 1.16 minute, M+H 249.0. 1H NMR (DMSO-d₆) Ppm 1.66 (s, 9H) 4.93 (d, 2H) 5.26 (t, 1H) 7.36-7.47 (m, 2H) 7.82 (dd, 1H) 8.61 (s, 1H).

### Step AG2: (1H-benzoimidazol-4-yl)-methanol

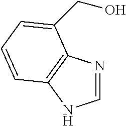 [SMILES: OCc1cccc2[nH]cnc12]

To a solution of 1H-Benzoimidazole-4-carboxylic acid methyl ester (Step AG3, 1.0 g, 5.68 mmol) in THF (56.8 mL) under argon was added LiAlH4 (1M in THF) (6.24 mL, 6.24 mmol), dropwise, causing a yellow coloration and a slight gas evolution. The reaction mixture was stirred at rt for 75 min. The medium was carefully quenched by addition of saturated aqueous NH₄Cl solution (50 mL). The slurry of aluminium salts was stirred for an hour at rt. The organic supernatant was decanted and the insoluble aluminium salts suspension was extracted with AcOEt (3×100 mL). The combined organic layers were dried over Na2SO4, filtered and concentrated under reduced pressure to give a colorless oil (600 mg, 71%). HPLC/MS (Method A) t_{R} 0.64 minute, M+H 149.0. 1H NMR (DMSO-d₆) Ppm 4.85 (br. s., 2H) 5.19 (br. s., 1H) 7.07-7.26 (m, 2H) 7.48 (d, J=7.34 Hz, 1H) 8.19 (s, 1H) 12.35-12.64 (m, 1H).

### Step AG3: 1H-Benzoimidazole-4-carboxylic acid methyl ester

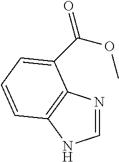

To a solution of 1H-benzimidazole-7-carboxylic acid (1 g, 6.17 mmol) in MeOH (20.56 mL) at 0° C. was added DMAP (0.151 g, 1.233 mmol) followed by DIC (1.057 mL, 6.78 mmol). The resulting reaction mixture was warmed up to rt and was stirred at rt for 4 hrs. The reaction mixture was then concentrated to dryness under vacuum. The crude was taken up in AcOEt (200 mL) and washed with saturated aqueous sodium bicarbonate solution (3×100 mL) and brine (100 mL). The organic layer was dried over Na2SO4, filtered and evaporated to give a clear oil. The residue was purified by chromatography on silica gel, using a 5% to 100% gradient of eluent B (EtOAc/MeOH/NH4OH:90/9/1) in eluent A (heptane/CH2Cl2:1/1), yielding the title product as a white solid, 550 mg (51%). HPLC/MS (Method A) t_{R} 0.70 minute, M+H 177.0. 1H NMR (DMSO-d₆) Ppm 3.95 (s, 3H) 7.32 (t, 1H) 7.86 (d, 1H) 7.97 (d, 1H) 8.31 (s, 1H) 12.57 (br. s., 1H)

### Intermediate AH: 4-Bromomethyl-benzotriazole-1-carboxylic acid tert-butyl ester

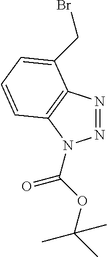 [SMILES: CC(C)(C)OC(=O)n1nnc2c(CBr)cccc21]

To a solution of 4-hydroxymethyl-benzotriazole-1-carboxylic acid tert-butyl ester (Step AH1, 1.58 g, 6.34 mmol) in CH2Cl2 (24 mL) was added triphenylphosphine (2.494 g, 9.51 mmol). The mixture was chilled to 0° C. then a solution of CBr₄ (3.15 g, 9.51 mmol) in CH2Cl2 (24.00 mL) was added dropwise and stirring was maintained at 0° C. for 1.5 hrs. The reaction micture was then evaporated to a crude residue which was purified by chromatography on silica gel, using a 3% to 15% gradient of eluent B (EtOAc) in eluent A (cyclohexane), yielding the title product as a clear oil, 1.59 g (80%). HPLC/MS (Method D) t_{R} 2.83 minute, M+H 312.314. 1H NMR (DMSO-d₆) Ppm 1.71 (s, 9H) 5.18 (s, 2H) 7.66 (d, 1H) 7.75 (t, 1H) 7.99 (d, 1H)

### Step AH1: 4-Hydroxymethyl-benzotriazole-1-carboxylic acid tert-butyl ester

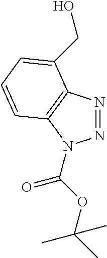 [SMILES: CC(C)(C)OC(=O)n1nnc2c(CO)cccc21]

To a well stirred suspension of (1H-benzotriazol-4-yl)-methanol (1.9 g, 12.74 mmol, synthesized as described in: Hurt, Clarence Ray; Pennell, Andrew. K.; Wright, John Jessen; Wang, Qiang; Leleti, Manmohan; Reddy; Thomas, William D.; Li, Yandong; Dragoli, Dean R. Substituted dihydropyridines as C5a receptor modulators and their preparation, pharmaceutical compositions and use in the treatment of diseases. WO2007051062) and sodium bicarbonate (2.140 g, 25.5 mmol) in MeCN (18 mL) and water (12 mL) was added a solution of Boc₂O (3.40 mL, 14.65 mmol) in MeCN (18 mL). Stirring was kept at RT for 2 hr. The reaction mixture was poured onto 30 ml of aqueous 0.5 M citric acid solution then extracted with EtOAc (3×50 mL). The combined organic layers were washed with brine then dried over MgSO4. The residue was purified by chromatography on silica gel, using a 10% to 40% gradient of eluent B (EtOAc) in eluent A (cyclohexane), yielding the title product as a clear oil, 2.97 g (94%). HPLC/MS (Method D) t_{R} 1.81 minute, M+H 250.0. 1H NMR (DMSO-d₆) Ppm 1.71 (s, 9H) 5.10 (d, 2H) 5.54 (t, 1H) 7.58 (d, 1H) 7.73 (t, 1H) 7.89 (d, 1H)

### Intermediate AK: 1-(4-Bromo-phenyl)-5-methyl-1H-benzoimidazol-2-ylamine

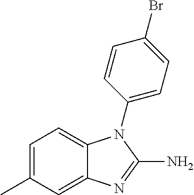 [SMILES: Cc1ccc2c(c1)nc(N)n2-c1ccc(Br)cc1]

N*1*-(4-Bromo-phenyl)-4-methyl-benzene-1,2-diamine (step AK1, 494 mg, 1.78 mmol) and cyanic bromide (191 mg, 1.78 mmol) were dissolved in acetonitrile (9 mL) and water (0.6 mL). The resulting mixture was stirred at rt for 16 h. The medium was evaporated to dryness and the residual purple oil was diluted with 1M aqueous NaOH (20 mL) and EtOAc (50 mL). The phases were separated. The aqueous phase was extracted with EtOAc (2×50 mL) and the combined organics were dried over Na2SO4 and concentrated to a crude brown oil. The oil was purified by chromatography on silica gel using a 10% to 100% gradient of eluent B (AcOEt+1% NH4OH) in eluent A (Heptane), leading to the title compound as a grey solid, 475 mg (88%). HPLC/MS (method A) t_{R} 1.02 minute, M+H 301.9-303.9.

### Step AK1: N*1*-(4-Bromo-phenyl)-4-methyl-benzene-1,2-diamine

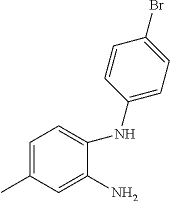 [SMILES: Cc1ccc(Nc2ccc(Br)cc2)c(N)c1]

A mixture of (4-Bromo-phenyl)-(4-methyl-2-nitro-phenyl)-amine (step AK2, 1.1 g, 3.5 mmol) and tin(II) chloride dihydrate (4.0 g, 17.5 mmol) in EtOH (11.7 mL) was heated to 70° C. for 120 minutes. The medium was then evaporated to dryness. The resulting crude oil was dissolved in EtOAc (100 mL) and treated with NaOH 1M (50 mL). The biphasic system was vigorously stirred for 20 minutes to allow complete precipitation of the tin salts. The medium was then filtered over diatomaceous earth through a sintered funnel and copiously washed with EtOAc. The biphasic filtrate was decanted, the aqueous layer was washed with EtOAc (100 mL) and the combined organics finally dried over Na₂SO₄. Evaporation of the volatiles yielded the title product, 494 mg (51%). HPLC/MS (method A) t_{R} 1.69 minute, M+H 276.9-278.9.

### Step AK2: (4-Bromo-phenyl)-(4-methyl-2-nitro-phenyl)-amine

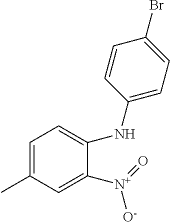 [SMILES: Cc1ccc(Nc2ccc(Br)cc2)c([N+](=O)[O-])c1]

A mixture of 1-bromo-4-iodo-benzene (1.00 g, 3.43 mmol), 4-amino-3-nitrotoluene (520 mg, 3.43 mmol), cesium carbonate (5.59 g, 17.20 mmol), palladium diacetate (23 mg, 0.10 mmol), triethylamine (0.48 mL, 3.43 mmol) and rac-BINAP (64 mg, 0.10 mmol) in toluene (7 mL) under argon was heated to reflux for 72 hours. The reaction mixture was then diluted in 20 mL of AcOEt, filtered over a pad of celite and concentrated under reduced pressure to give a dark solid. The red solid was taken up in EtOAc (100 mL) and water (50 mL). The phases were separated, the aqueous portion further extracted with EtOAc (2×100 mL), the combined organic layers dried over sodium sulfate and concentrated. The crude product was purified by chromatography on silica gel, using a 10% to 100% gradient of eluent B (MTBE+5% (7N NH3 in MeOH)) in eluent A (heptane), leading to the title compound as a brown solid, 1.19 g (89%). HPLC/MS (method D) t_{R} 1.72 minute (85% UV diode array purity), M+H 306.9-308.9.

### Intermediate AL: 1-(4-Bromo-phenyl)-7-chloro-1H-benzoimidazol-2-ylamine

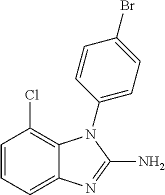 [SMILES: Nc1nc2cccc(Cl)c2n1-c1ccc(Br)cc1]

N*2*-(4-Bromo-phenyl)-3-chloro-benzene-1,2-diamine (step AL1, 200 mg, 0.638 mmol) was dissolved in MeCN (3.2 mL) and BrCN (135 mg, 1.277 mmol) was added. The resulting reaction mixture was shaken for 4 hours at rt before the addition of more BrCN (135 mg, 1.277 mmol). The temperature was raised to 50° C. and the mixture was stirred for 24 hours. The reaction mixture was cooled down to rt and treated with 1N aqueous sodium hydroxide solution (20 mL). The resulting aqueous mixture was extracted with CH₂Cl₂ (3×20 mL). The combined organic layers were then evaporated to dryness. The crude residue was purified by chromatography on amino-derivatized silica gel (Biotage cartridge 25+S-KP-NH), using a 0% to 100% gradient of eluent B (EtOAc) in eluent A (heptane), leading to the title compound as a solid, 120 mg (58%). HPLC/MS (method A) t_{R} 1.10 minute, M+H 321.9-323.9. ¹H NMR (DMSO-d₆) Ppm 6.34 (bs, 2H) 6.84 (d, 1H) 6.99 (t, 1H) 7.18 (d, 1H) 7.41 (d, 2H) 7.74 (d, 2H).

### Step AL1: N*2*-(4-Bromo-phenyl)-3-chloro-benzene-1,2-diamine

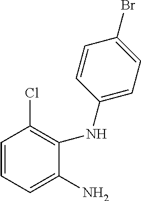 [SMILES: Nc1cccc(Cl)c1Nc1ccc(Br)cc1]

(4-Bromo-phenyl)-(2-chloro-6-nitro-phenyl)-amine (step AL2, 241 mg, 0.662 mmol) was dissolved in EtOH (3.3 mL) and SnCl2.2H2O (374 mg, 1.655 mmol) was added. The resulting reaction mixture was stirred for 16 hours at 80° C., after which time SnCl2.2H2O (374 mg, 1.655 mmol) was added. The reaction was stirred further for 2 hours at 80° C., after which time the reaction was allowed to cool down to rt. The reaction mixture was quenched with saturated aqueous sodium bicarbonate solution (10 mL) and the resulting suspension was filtered through a pad of celite. The cake was washed with EtOAc (3×10 mL). The layers were separated and the organic portion was further extracted with water (2×10 mL) and brine (10 mL). The organic phase was then dried over Na2SO4, filtered and evaporated to dryness to furnish the title compound as a solid, 200 mg (96%). HPLC/MS (method A) t_{R} 1.62 minute, M+H 296.9-298.9. ¹H NMR (DMSO-d₆) Ppm 5.13 (bs, 2H) 6.42 (d, 2H) 6.70 (dd, 2H) 6.97 (t, 1H) 7.23 (d, 1H) 7.40 (s, 1H).

### Step AL2: (4-Bromo-phenyl)-(2-chloro-6-nitro-phenyl)-amine

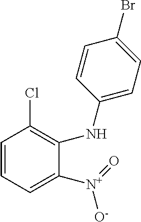 [SMILES: O=[N+]([O-])c1cccc(Cl)c1Nc1ccc(Br)cc1]

3-Chloro-2-fluoro-1-nitro-benzene (100 μl, 0.851 mmol), 4-bromoaniline (146 mg, 0.851 mmol) and NEt₃ (119 μl, 0.851 mmol) were mixed together. The vessel was sealed and the reaction was stirred for 1 hour at 180° C. under microwave irradiation. The reaction was quenched with 1N aqueous HCl solution (10 mL). This aqueous suspension was then extracted with EtOAc (10 mL). The resulting organic portion was washed with 1N aqueous HCl solution (2×10 mL), dried over Na2SO4, filtered and evaporated to dryness to furnish the title compound, 241 mg (90% UV diode array purity, 78%). HPLC/MS (method A) t_{R} 1.73 minute (90% UV diode array purity), M+H 325.0-326.9.

### Intermediate AM: 1-(4-Bromo-phenyl)-6-methoxy-1H-benzoimidazol-2-ylamine

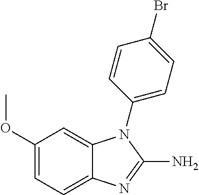 [SMILES: COc1ccc2nc(N)n(-c3ccc(Br)cc3)c2c1]

To a solution of N*2*-(4-Bromo-phenyl)-4-methoxy-benzene-1,2-diamine (step AM1, 2.3 g, 9.21 mmol) in acetonitrile (17.13 mL) and water (1.290 mL) was added cyanogen bromide (0.976 g, 9.21 mmol) at RT. The reaction mixture was stirred for 16 hours at rt before concentration under reduced pressure to a dark oil. This residue was taken up in AcOEt (200 mL) and washed with saturated aqueous sodium bicarbonate solution (150 mL). The organic layer was dried over Na2SO4, filtered and concentrated to a dark solid. The crude product was purified by chromatography on silica gel, using a 0% to 100% gradient of eluent B (EtOAc/MeOH/NH₄OH:89/10/1) in eluent A (heptane/CH₂Cl₂:1/1), leading to the title compound as a deep red solid, 1.83 g (72%). HPLC/MS (method A) t_{R} 0.79 minute, M+H 275.0-277.0. ¹H NMR (DMSO-d₆) Ppm 3.67 (s, 3H) 6.26 (s, 2H) 6.48 (d, 1H) 6.65 (dd, 1H) 7.12 (d, 1H) 7.76 (d, 1H) 8.03 (dd, 1H) 8.57 (d, 1H).

### Step AM1: N*2*-(4-Bromo-phenyl)-4-methoxy-benzene-1,2-diamine

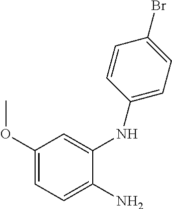 [SMILES: COc1ccc(N)c(Nc2ccc(Br)cc2)c1]

To a suspension of (4-Bromo-phenyl)-(5-methoxy-2-nitro-phenyl)-amine (step AM2, 35.5 g, 133 mmol) in EtOH (59.6 ml) was added tin(II) chloride dihydrate (12.10 g, 53.6 mmol) and the mixture was stirred for 3 hours at 70° C. The medium was allowed to cool down to rt. The reaction mixture was treated with 1N aqueous sodium hydroxide solution (143 mL, 143 mmol) and filtered over diatomeceous earth. The solid cake was washed with MeOH (2×100 mL). The filtrate was concentrated to a brown essentially aqueous suspension which was diluted with AcOEt (300 mL). The phases were separated and the aqueous layer was backextracted with AcOEt (2×200 mL). The combined organic phases were dried over Na₂SO₄, filtered and concentrated to give the title product as a brown solid, 2.3 g (52%). HPLC/MS (method A) t_{R} 0.85 minute, M+H 250.0-252.0.

### Step AM2: (4-Bromo-phenyl)-(5-methoxy-2-nitro-phenyl)-amine

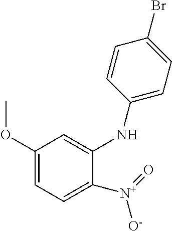 [SMILES: COc1ccc([N+](=O)[O-])c(Nc2ccc(Br)cc2)c1]

NaH 60% in mineral oil (46.7 g, 1167 mmol) was suspended in THF (194 mL) and the white suspension was stirred under argon at 0° C. To this mixture was added a solution of 2-chloro-5-aminopyridine (30.0 g, 233 mmol) in THF (194 mL) causing a clear red suspension. When the addition was finished, the reaction mixture was allowed to warm up to rt and was stirred for 30 minutes. The mixture was then cooled to 0° C. and a solution of 2,4-difluoro-1-nitro-benzene (51.2 mL, 467 mmol) in THF (389 mL) was added slowly at 0° C. The suspension was allowed to warm up to rt under stirring over the course of 1 hour. The reaction was cooled again to 0° C. and MeOH (200 mL) was added dropwise (strong exotherm) under stirring, followed by water (150 mL). The mixture was concentrated under vacuum and the residual suspension was partitioned between diethyl ether (1000 mL) and 1N aqueous HCl solution (500 mL). The biphasic medium was stirred at rt for 10 minutes, after which time diethyl ether (500 mL) and 1N aqueous HCl solution (500 mL) were added, causing a precipitation to occur. The solid was filtered, washed with diethyl ether and dried under vacuum to afford the title product as a dark yellow powder, 54.6 g (84%). HPLC/MS (method A) t_{R} 1.44 minute, M+H 280.0-282.0. ¹H NMR (Acetone-d₆) Ppm 3.85 (s, 3H) 6.56 (dd, 1H) 6.66 (d, 1H) 7.53 (d, 1H) 7.96 (dd, 1H) 8.20 (d, 1H) 8.51 (d, 1H) 9.62 (br. s., 1H).

### III Chemical Synthesis—Compounds According to the Invention

### Example 1

### N-{5-Chloro-4′-[3-(2-chloro-benzyl)-2-imino-2,3-dihydro-benzoimidazol-1-yl]-biphenyl-2-yl}-acetamide

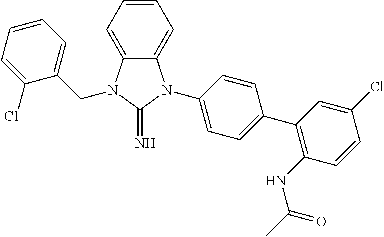 [SMILES: CC(=O)Nc1ccc(Cl)cc1-c1ccc(-n2c(=N)n(Cc3ccccc3Cl)c3ccccc32)cc1]

Intermediate M (70 mg, 0.170 mmol), 2-acetamido-5-chlorophenyl boronic acid pinacol ester (55.1 mg, 0.187 mmol) and Pd(Ph3P)4 (9.80 mg, 8.48 μmol) were suspended in DME (848 μL) and 2M aqueous Na2CO3 solution (424 μL, 0.848 mmol). The resulting reaction mixture was stirred for 17 minutes at 150° C. under microwave irradiation. The reaction mixture was then diluted with EtOAc (2 mL) and filtered through a pad of diatomaceous earth which was rinsed with EtOAc (3×3 mL). The resulting organic filtrate was evaporated to dryness and the crude product purified by reverse-phase preparative HPLC (Method E). Product-containing fractions were pooled and evaporated to dryness. The residue was dissolved in a minimum of MeOH and flowed through a StratoSphere PL-carbonate cartridge (Polymer laboratories, Varian inc.) to remove residual trifluoroacetic acid. Evaporation of the resulting filtrate yielded the title compound as a white solid, 26 mg (30%). HPLC/MS (Method D) t_{R} 2.32 min, M+H 500.8. 1H NMR (dmso-d6) Ppm 1.95 (s, 3H) 5.23 (s, 2H) 6.79-7.02 (m, 3H) 7.03-7.17 (m, 1H) 7.19-7.40 (m, 2H) 7.42-7.51 (m, 2H) 7.51-7.75 (m, 8H) 9.43 (s, 1H)

### Example 2

### N-{4′-[3-(2-Chloro-benzyl)-2-imino-2,3-dihydro-benzoimidazol-1-yl]-biphenyl-2-yl}-acetamide

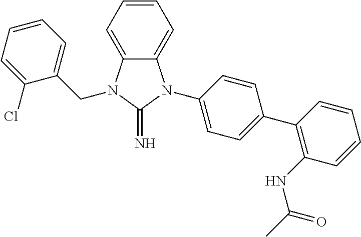 [SMILES: CC(=O)Nc1ccccc1-c1ccc(-n2c(=N)n(Cc3ccccc3Cl)c3ccccc32)cc1]

Intermediate M (200.0 mg, 0.485 mmol), 2-acetamidophenylboronic acid (95.0 mg, 0.533 mmol) and Pd(Ph3P)4 (28.0 mg, 0.024 mmol) were suspended in DME (2400 μL) and 2M aqueous Na2CO3 solution (1200 μl, 2.423 mmol). The resulting reaction mixture was stirred for 18 hours at 90° C. The reaction mixture was then diluted with MeOH (3 mL) and filtered through a pad of diatomaceous earth which was rinsed with EtOAc (3×10 mL). The resulting organic filtrate was evaporated to dryness. The crude medium was taken up in EtOAc (50 mL) and washed successively with aqueous 0.1 N sodium hydroxide solution (2×25 mL) and brine (25 mL). The organic phase was dried over Na2SO4 and concentrated to a crude solid. The crude product was purified by reverse-phase preparative HPLC (Method E). Product-containing fractions were pooled and evaporated to dryness. The residue was dissolved in CH2Cl2 (10 mL), washed with saturated sodium bicarbonate solution (2×10 mL), dried again over Na2SO4 and finally evaporated to dryness to afford the title product as a grey solid, 82 mg (36%). HPLC/MS (Method A) t_{R} 1.19 min, M+H 467.0. ¹H NMR (dmso-d6) Ppm 1.94 (s, 3H) 5.22 (s, 2H) 6.78-7.00 (m, 4H) 7.03-7.19 (m, 1H) 7.26-7.36 (m, 3H) 7.37-7.47 (m, 2H) 7.49-7.74 (m, 7H) 9.37 (br. s., 1H).

### Example 3

### N-{4′-[3-(2-Chloro-benzyl)-2-imino-2,3-dihydro-benzoimidazol-1-yl]-biphenyl-4-yl}-acetamide

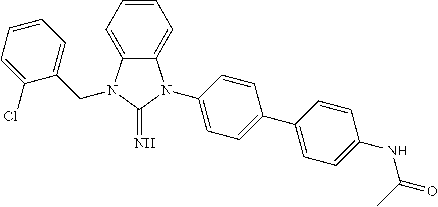 [SMILES: CC(=O)Nc1ccc(-c2ccc(-n3c(=N)n(Cc4ccccc4Cl)c4ccccc43)cc2)cc1]

Intermediate M (50 mg, 0.121 mmol), 4-Acetamidophenylboronic acid (23.7 mg, 0.133 mmol) and Pd(Ph3P)₄ (7.1 mg, 6.0 μmol) were suspended in DME (605 μL) and 2M aqueous Na2CO3 solution (300 μL, 0.600 mmol). The resulting reaction mixture was stirred for 17 minutes at 150° C. under microwave irradiation. The reaction mixture was then diluted with MeOH (2 mL) and filtered through a pad of diatomaceous earth which was rinsed with EtOAc (2×2 mL). The resulting organic filtrate was evaporated to dryness and the crude product purified by reverse-phase preparative HPLC (Method E, gradient from 5% MeCN (+0.1% TFA) in water (+0.1% TFA) to 100% over 14 minutes). Product-containing fractions were pooled and evaporated to dryness to furnish the title compound as a TFA salt, 19 mg (27%). HPLC/MS (Method A) t_{R} 1.22 min, M+H 466.9. ¹H-NMR (dmso-d6) Ppm 2.09 (s, 3H) 5.58 (s, 2H) 7.07-7.21 (m, 2H) 7.27-7.37 (m, 3H) 7.38-7.49 (m, 2H) 7.62 (d, 1H) 7.72-7.86 (m, 6H) 8.03 (d, 2H) 8.92 (s, 2H) 10.12 (s, 1H).

The following examples 3.1 to 3.25 were synthesized in a manner analogous to that used for the synthesis of example 3, from intermediate M and various aryl boronic acids or boronate esters.

| Ex. | structure | name | HPLC* | MS** | 1H NMR*** |
|---|---|---|---|---|---|
| 3.1 | 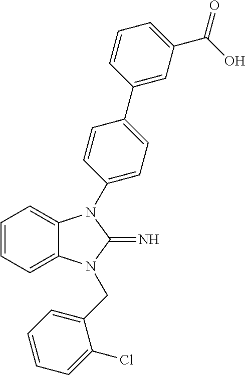 [SMILES: N=c1n(Cc2ccccc2Cl)c2ccccc2n1-c1ccc(-c2cccc(C(=O)O)c2)cc1] | 4′-[3-(2-Chloro- benzyl)-2-imino-2,3- dihydro- benzoimidazol-1-yl]- biphenyl- 3-carboxylic acid | 1.23 (A) | 453.9 | |
| 3.2 | 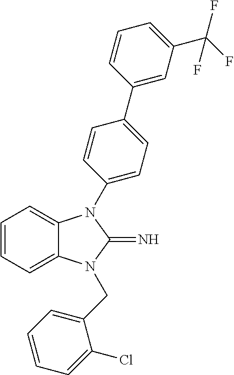 [SMILES: N=c1n(Cc2ccccc2Cl)c2ccccc2n1-c1ccc(-c2cccc(C(F)(F)F)c2)cc1] | 1-(2-Chloro-benzyl)- 3-(3′-trifluoro methyl-biphenyl-4- yl)-1,3-dihydro- benzoimidazol-2- ylideneamine | 1.48 (A) | 478.0 | |
| 3.3 | 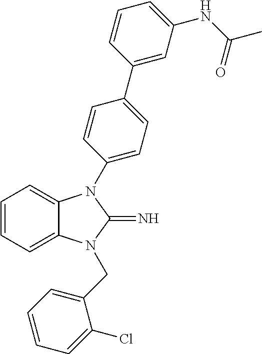 [SMILES: CC(=O)Nc1cccc(-c2ccc(-n3c(=N)n(Cc4ccccc4Cl)c4ccccc43)cc2)c1] | N-{4′-[3-(2-Chloro- benzyl)-2-imino- 2,3-dihydro- benzoimidazol-1-yl]- biphenyl-3-yl}- acetamide | 1.21 (A) | 466.9 | |
| 3.4 | 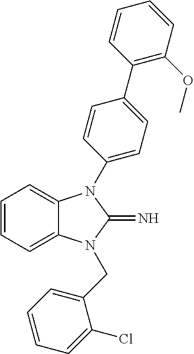 [SMILES: COc1ccccc1-c1ccc(-n2c(=N)n(Cc3ccccc3Cl)c3ccccc32)cc1] | 1-(2-Chloro-benzyl)- 3-(2′-methoxy- biphenyl-4-yl)-1,3- dihydro-benzoimidazol- 2-ylideneamine | 1.40 (A) | 440.0 | |
| 3.5 | 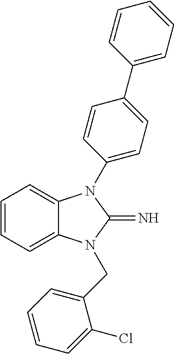 [SMILES: N=c1n(Cc2ccccc2Cl)c2ccccc2n1-c1ccc(-c2ccccc2)cc1] | 1-Biphenyl-4-yl-3- (2-chloro-benzyl)- 1,3-dihydro- benzoimidazol-2- ylideneamine | 1.39 (A) | 410.0 | |
| 3.6 | 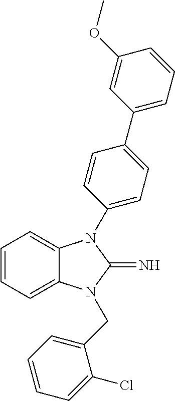 [SMILES: COc1cccc(-c2ccc(-n3c(=N)n(Cc4ccccc4Cl)c4ccccc43)cc2)c1] | 1-(2-Chloro-benzyl)- 3-(3′-methoxy- biphenyl-4-yl)-1,3- dihydro-benzoimidazol- 2-ylideneamine | 1.39 (A) | 440.0 | |
| 3.7 | 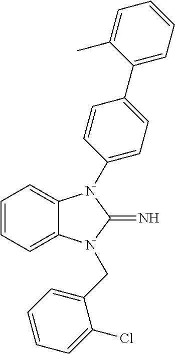 [SMILES: Cc1ccccc1-c1ccc(-n2c(=N)n(Cc3ccccc3Cl)c3ccccc32)cc1] | 1-(2-Chloro-benzyl)- 3-(2′-methyl- biphenyl-4-yl)-1,3- dihydro-benzoimidazol- 2-ylideneamine | 1.43 (A) | 424.0 | |
| 3.8 | 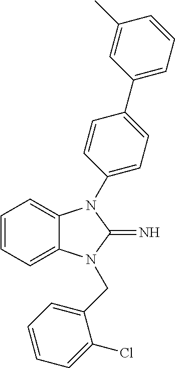 [SMILES: Cc1cccc(-c2ccc(-n3c(=N)n(Cc4ccccc4Cl)c4ccccc43)cc2)c1] | 1-(2-Chloro-benzyl)- 3-(3′-methyl- biphenyl-4-yl)-1,3- dihydro-benzoimidazol- 2-ylideneamine | 1.45 (A) | 424.0 | Ppm 2.44 (s, 3H) 5.59 (s, 2H) 7.15 (dd, 2H) 7.27-7.65 (m, 10H) 7.85 (d, 2H) 8.05 (d, 2H) 8.94 (s, 2H) |
| 3.9 | 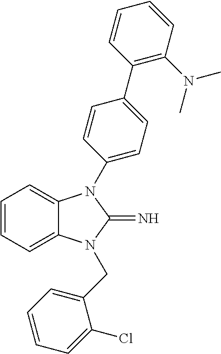 [SMILES: CN(C)c1ccccc1-c1ccc(-n2c(=N)n(Cc3ccccc3Cl)c3ccccc32)cc1] | {4′-[3-(2-Chloro- benzyl)-2-imino-2,3- dihydro- benzoimidazol-1-yl]- biphenyl- 2-yl}-dimethyl- amine | 1.40 (A) | 227.0 (M + 2H)/ 2 | |
| 3.10 | 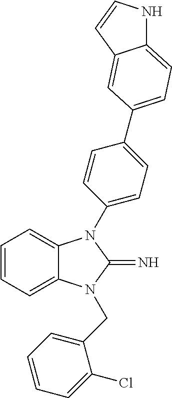 [SMILES: N=c1n(Cc2ccccc2Cl)c2ccccc2n1-c1ccc(-c2ccc3[nH]ccc3c2)cc1] | 1-(2-Chloro-benzyl)- 3-[4-(1H-indol- 5-yl)-phenyl]-1,3- dihydro-benzoimidazol- 2-ylideneamine | 1.33 (A) | 449.0 | |
| 3.11 | 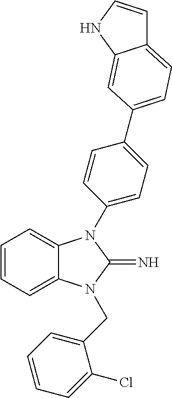 [SMILES: N=c1n(Cc2ccccc2Cl)c2ccccc2n1-c1ccc(-c2ccc3cc[nH]c3c2)cc1] | 1-(2-Chloro-benzyl)- 3-[4-(1H-indol- 6-yl)-phenyl]-1,3- dihydro-benzoimidazol- 2-ylideneamine | 1.36 (A) | 449.0 | |
| 3.12 | 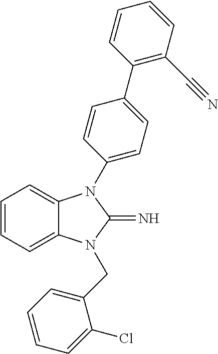 [SMILES: N#Cc1ccccc1-c1ccc(-n2c(=N)n(Cc3ccccc3Cl)c3ccccc32)cc1] | 4′-[3-(2-Chloro- benzyl)-2-imino-2,3- dihydro- benzoimidazol-1-yl]- biphenyl- 2-carbonitrile | 1.30 (A) | 434.9 | |
| 3.13 | 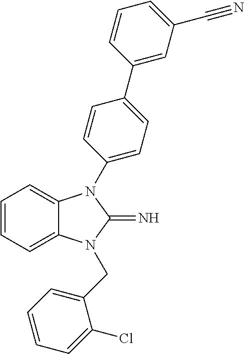 [SMILES: N#Cc1cccc(-c2ccc(-n3c(=N)n(Cc4ccccc4Cl)c4ccccc43)cc2)c1] | 4′-[3-(2-Chloro- benzyl)-2-imino-2,3- dihydro- benzoimidazol-1-yl]- biphenyl- 3-carbonitrile | 1.31 (A) | 434.9 | |
| 3.14 | 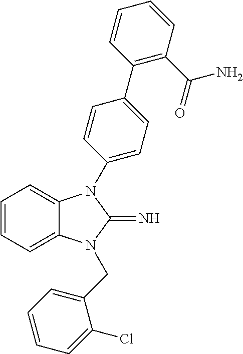 [SMILES: N=c1n(Cc2ccccc2Cl)c2ccccc2n1-c1ccc(-c2ccccc2C(N)=O)cc1] | 4′-[3-(2-Chloro- benzyl)-2-imino-2,3- dihydro- benzoimidazol-1-yl]- biphenyl- 2-carboxylic acid amide | 1.14 (A) | 452.9 | |
| 3.15 | 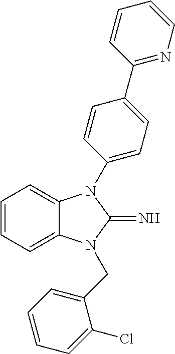 [SMILES: N=c1n(Cc2ccccc2Cl)c2ccccc2n1-c1ccc(-c2ccccn2)cc1] | 1-(2-Chloro-benzyl)- 3-(4-pyridin-2- yl-phenyl)-1,3- dihydro- benzoimidazol- 2-ylideneamine | 1.23 (A) | 411.9 | |
| 3.16 | 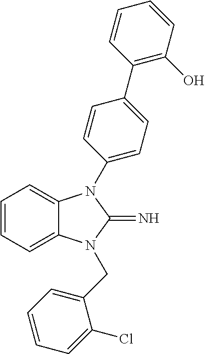 [SMILES: N=c1n(Cc2ccccc2Cl)c2ccccc2n1-c1ccc(-c2ccccc2O)cc1] | 4′-[3-(2-Chloro- benzyl)-2-imino-2,3- dihydro- benzoimidazol-1-yl]- biphenyl- 2-ol | 1.18 (G) | 426 | |
| 3.17 | 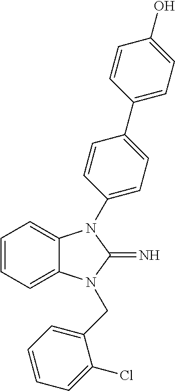 [SMILES: N=c1n(Cc2ccccc2Cl)c2ccccc2n1-c1ccc(-c2ccc(O)cc2)cc1] | 4′-[3-(2-Chloro- benzyl)-2-imino-2,3- dihydro- benzoimidazol-1-yl]- biphenyl- 4-ol | 1.14 (G) | 426 | |
| 3.18 | 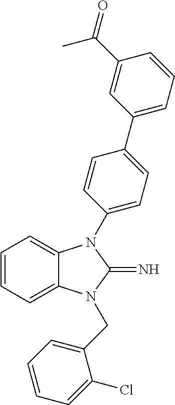 [SMILES: CC(=O)c1cccc(-c2ccc(-n3c(=N)n(Cc4ccccc4Cl)c4ccccc43)cc2)c1] | 1-{4′-[3-(2-Chloro- benzyl)-2-imino- 2,3-dihydro- benzoimidazol-1-yl]- biphenyl-3-yl}- ethanone | 1.20 (G) | 452 | |
| 3.19 | 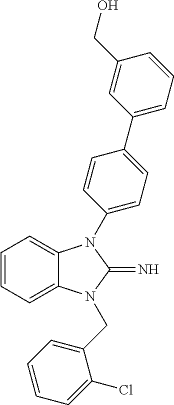 [SMILES: N=c1n(Cc2ccccc2Cl)c2ccccc2n1-c1ccc(-c2cccc(CO)c2)cc1] | {4′-[3-(2-Chloro- benzyl)-2-imino-2,3- dihydro- benzoimidazol-1-yl]- biphenyl-3-yl}- methanol | 1.11 (G) | 440 | |
| 3.20 | 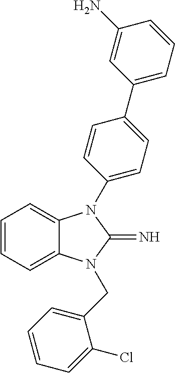 [SMILES: N=c1n(Cc2ccccc2Cl)c2ccccc2n1-c1ccc(-c2cccc(N)c2)cc1] | 4′-[3-(2-Chloro- benzyl)-2-imino-2,3- dihydro- benzoimidazol-1-yl]- biphenyl-3-ylamine | 0.96 (G) | 425 | |
| 3.21 | 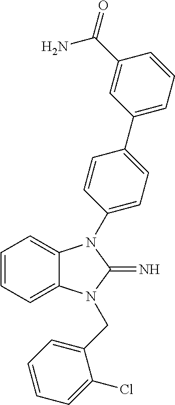 [SMILES: N=c1n(Cc2ccccc2Cl)c2ccccc2n1-c1ccc(-c2cccc(C(N)=O)c2)cc1] | 4′-[3-(2-Chloro- benzyl)-2-imino-2,3- dihydro- benzoimidazol-1-yl]- biphenyl-3- carboxylic acid amide | 1.05 (G) | 453 | |
| 3.22 | 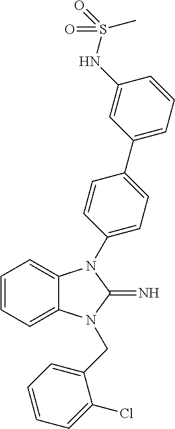 [SMILES: CS(=O)(=O)Nc1cccc(-c2ccc(-n3c(=N)n(Cc4ccccc4Cl)c4ccccc43)cc2)c1] | N-{4′-[3-(2-Chloro- benzyl)-2-imino- 2,3-dihydro- benzoimidazol-1-yl]- biphenyl-3-yl}- methanesulfon- amide | 1.13 (G) | 503 | |
| 3.23 | 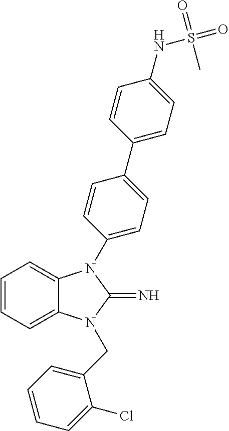 [SMILES: CS(=O)(=O)Nc1ccc(-c2ccc(-n3c(=N)n(Cc4ccccc4Cl)c4ccccc43)cc2)cc1] | N-{4′-[3-(2-Chloro- benzyl)-2-imino- 2,3-dihydro- benzoimidazol-1-yl]- biphenyl-4-yl}- methanesulfon- amide | 1.13 (G) | 503 | |
| 3.24 | 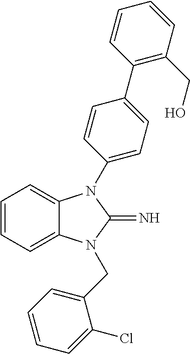 [SMILES: N=c1n(Cc2ccccc2Cl)c2ccccc2n1-c1ccc(-c2ccccc2CO)cc1] | {4′-[3-(2-Chloro- benzyl)-2-imino-2,3- dihydro- benzoimidazol-1-yl]- biphenyl-2-yl}- methanol | 1.29 (A) | 440.1 | Ppm 4.53 (s, 2H) 5.27 (bs, 1H) 5.59 (s, 2H) 7.16 (m, 2H) 7.30-7.50 (m, 8H) 7.65 (m, 2H) 7.80 (m, 4H) 8.95 (s, 2H) |
| 3.25 | 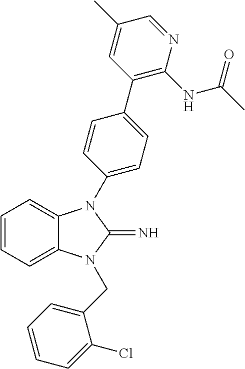 [SMILES: CC(=O)Nc1ncc(C)cc1-c1ccc(-n2c(=N)n(Cc3ccccc3Cl)c3ccccc32)cc1] | N-(3-{4-[3-(2- Chloro-benzyl)-2- imino-2,3-dihydro- benzoimidazol-1-yl]- phenyl}-5-methyl- pyridin-2-yl)- acetamide | 1.22 (A) | 482.0; (M + 2H)/ 2 241.5 | |

*t_{R} [min] (method); **M + H (or specified); ***d⁶-dmso

### Example 4

### 4′-[3-(2-Chloro-benzyl)-2-imino-2,3-dihydro-benzoimidazol-1-yl]-biphenyl-2-ylamine

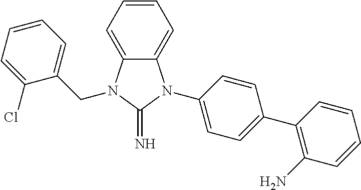 [SMILES: N=c1n(Cc2ccccc2Cl)c2ccccc2n1-c1ccc(-c2ccccc2N)cc1]

Intermediate M (1850 mg, 4.46 mmol), 2-Aminophenylboronic acid pinacol ester (1100 mg, 4.91 mmol) and Pd(Ph3P)₄ (263 mg, 223 μmol) were suspended in DME (22 mL) and 2M aqueous Na2CO3 solution (11 mL, 22.0 mmol). The resulting reaction mixture was stirred for 17 minutes at 150° C. under microwave irradiation. The reaction mixture was then diluted with MeOH (40 mL) and filtered through a pad of diatomaceous earth which was rinsed with EtOAc (2×30 mL). The resulting organic filtrate was evaporated to dryness and the crude product purified by chromatography on silica gel, using a 20% to 80% gradient of eluent B (EtOAc+1% NH4OH) in eluent A (heptane/Ch2Cl2:1/1), leading to the title compound as an orange gum, 1280 mg (68%). HPLC/MS (method A) t_{R} 1.27 minute, M+H 425.0. 1H NMR (dmso-d6) Ppm 4.91 (bs, 2H) 5.23 (s, 2H) 6.68 (t, 1H) 6.92 (d, 1H) 7.08 (m, 4H) 7.10 (m, 3H) 7.32 (m, 2H) 7.54 (d, 1H) 7.64 (m, 5H).

### Example 5

### N-{4′-[3-(2-Chloro-benzyl)-2-imino-2,3-dihydro-benzoimidazol-1-yl]-biphenyl-2-yl}-3-cyano-benzamide

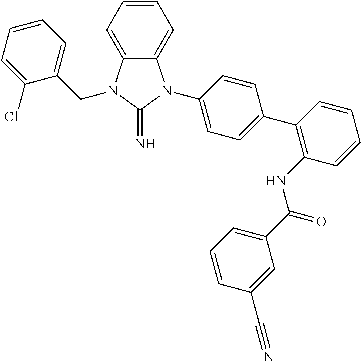 [SMILES: N#Cc1cccc(C(=O)Nc2ccccc2-c2ccc(-n3c(=N)n(Cc4ccccc4Cl)c4ccccc43)cc2)c1]

To a solution of 4′-[3-(2-Chloro-benzyl)-2-imino-2,3-dihydro-benzoimidazol-1-yl]-biphenyl-2-ylamine (example 4, 40.0 mg, 0.094 mmol) and pyridine (13.4 μL, 0.187 mmol) in CH2Cl2 (313 μL) was added 3-Cyanobenzoyl chloride (16.6 mg, 0.098 mmol) and the resulting mixture stirred at rt for one hour. The medium was then diluted with CH2Cl2 (5 mL), washed with 0.1 N aqueous sodium hydroxide solution (3×5 mL), brine (5 mL), dried over Na2SO4 and finally evaporated to dryness under reduced pressure. The crude product was purified by reverse-phase preparative HPLC (Method E). Product-containing fractions were pooled and evaporated to dryness to furnish the title compound as a TFA salt, 16 mg (26%). HPLC/MS (Method A) t_{R} 1.33 min, M+H 554.0. ¹H NMR (dmso-d6) Ppm 5.57 (s, 2H) 6.77 (d, 1H) 7.14 (d, 1H) 7.25-7.40 (m, 5H) 7.50-7.65 (m, 5H) 7.71-7.81 (m, 5H) 8.06 (d, 1H) 8.15 (d, 1H) 8.25 (s, 1H) 8.95 (s, 2H) 10.27 (s, 1H).

The following examples 5.1 to 5.20 were synthesized in a manner analogous to that used for the synthesis of example 5, from example 4 and various aryl acyl chlorides.

| Ex. | structure | name | HPLC* | MS** | 1H NMR*** |
|---|---|---|---|---|---|
| 5.1 | 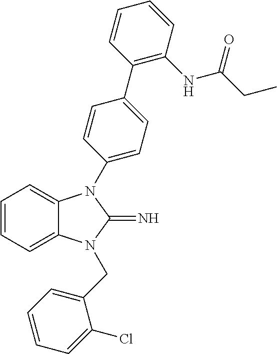 [SMILES: CCC(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}- propionamide | 1.25 (A) | 481.0 | |
| 5.2 | 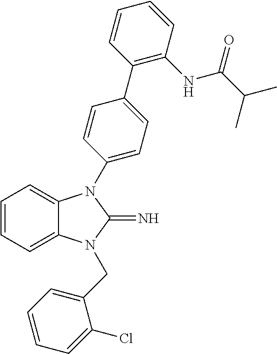 [SMILES: CC(C)C(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}- isobutyramide | 1.25 (A) | 495.0 | |
| 5.3 | 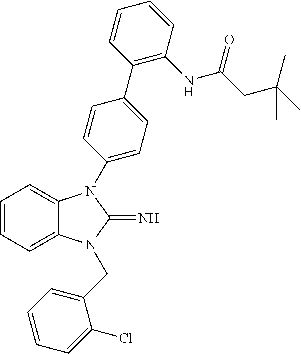 [SMILES: CC(C)(C)CC(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}-3,3- dimethyl- butyramide | 1.37 (A) | 523.0 | |
| 5.4 | 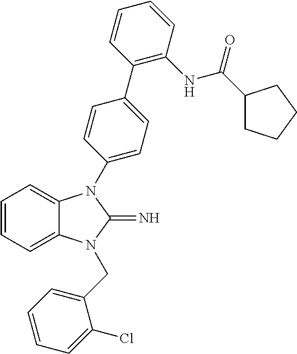 [SMILES: N=c1n(Cc2ccccc2Cl)c2ccccc2n1-c1ccc(-c2ccccc2NC(=O)C2CCCC2)cc1] | Cyclopentane- carboxylic acid {4′-[3-(2-chloro- benzyl)-2- imino-2,3- dihydro- benzoimidazol- 1-yl]-biphenyl- 2-yl}-amide | 1.33 (A) | 521.0 | |
| 5.5 | 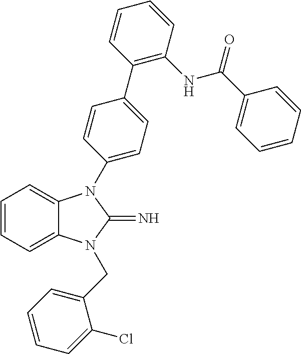 [SMILES: N=c1n(Cc2ccccc2Cl)c2ccccc2n1-c1ccc(-c2ccccc2NC(=O)c2ccccc2)cc1] | N-{4′-[3-(2- Chloro-benzyl)- 2-imino- 2,3-dihydro- benzoimidazol- 1-yl]-biphenyl- 2-yl}- benzamide | 1.32 (A) | 529.0 | |
| 5.6 | 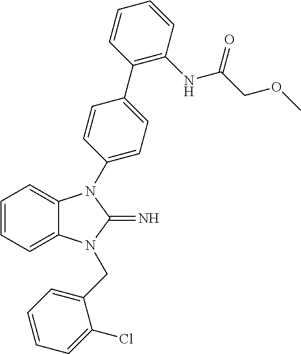 [SMILES: COCC(=O)Nc1ccccc1-c1ccc(-n2c(=N)n(Cc3ccccc3Cl)c3ccccc32)cc1] | N-{4′-[3-(2- Chloro-benzyl)- 2-imino-2,3- dihydro- benzoimidazol- 1-yl]-biphenyl- 2-yl}-2- methoxy- acetamide | 1.23 (A) | 497.0 | |
| 5.7 | 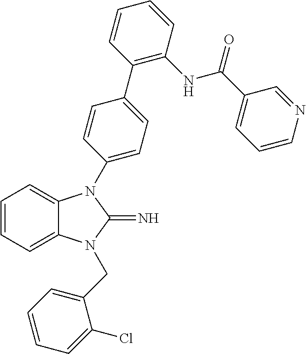 [SMILES: N=c1n(Cc2ccccc2Cl)c2ccccc2n1-c1ccc(-c2ccccc2NC(=O)c2cccnc2)cc1] | N-{4′-[3-(2- Chloro-benzyl)- 2-imino-2,3- dihydrobenzo- imidazol-1-yl]- biphenyl-2-yl}- nicotinamide | 1.18 (A) | 530.0 | Ppm 5.56 (s, 2H) 6.76 (d, 1H) 7.15 (d, 1H) 7.25-7.65 (m, 10H) 7.79 (m, 4H) 8.21 (bd, 1H) 8.74 (m, 1H) 8.95 (m, 3H) 10.28 (s, 1H) |
| 5.8 | 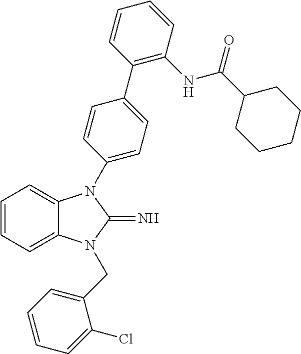 [SMILES: N=c1n(Cc2ccccc2Cl)c2ccccc2n1-c1ccc(-c2ccccc2NC(=O)C2CCCCC2)cc1] | Cyclohexane- carboxylic acid {4′-[3-(2-chloro- benzyl)-2- imino-2,3- dihydro- benzoimidazol- 1-yl]-biphenyl- 2-yl}-amide | 1.36 (A) | 535.0 | Ppm 1.00-1.50 (m, 6H) 1.50-1.75 (m, 4H) 2.24 (t, 1H) 5.58 (s, 2H) 6.99 (d, 1H) 7.18 (d, 1H) 7.30- 7.80 (m, 14H) 8.96 (s, 2H) 9.29 (s, 1H) |
| 5.9 | 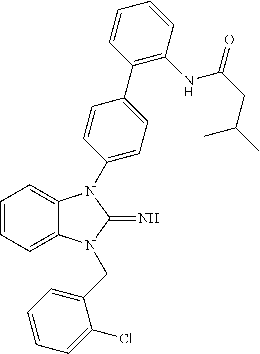 [SMILES: CC(C)CC(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}-3-methyl- butyramide | 1.30 (A) | 509.0 | Ppm 0.88 (d, 6H) 2.00 (q, 1H) 2.10 (d, 2H) 5.61 (s, 2H) 7.04 (d, 1H) 7.18 (d, 1H) 7.30-7.85 (m, 14H) 8.92 (s, 2H) 9.31 (s, 1H) |
| 5.10 | 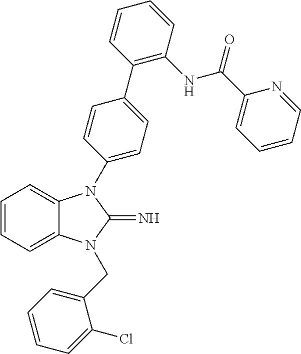 [SMILES: N=c1n(Cc2ccccc2Cl)c2ccccc2n1-c1ccc(-c2ccccc2NC(=O)c2ccccn2)cc1] | Pyridine-2- carboxylic acid {4′-[3-(2-chloro- benzyl)-2- imino-2,3- dihydro- benzoimidazol- 1-yl]-biphenyl- 2-yl}-amide | 1.36 (A) | 530.0 | Ppm 5.58 (s, 2H) 6.98 (d, 1H) 7.18 (s, 1H) 7.30-7.70 (m, 10H) 7.88 (s, 4H) 8.08 (t, 1H) 8.15 (d, 1H) 8.27 (d, 1H) 8.54 (d, 1H) 9.01 (s, 2H) 10.40 (s, 1H) |
| 5.11 | 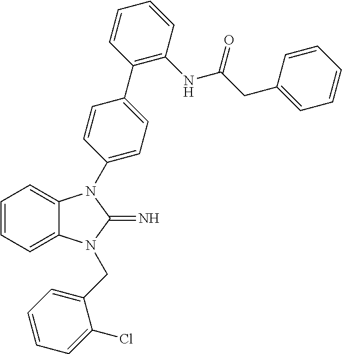 [SMILES: N=c1n(Cc2ccccc2Cl)c2ccccc2n1-c1ccc(-c2ccccc2NC(=O)Cc2ccccc2)cc1] | N-{4′-[3-(2- Chloro-benzyl)- 2-imino- 2,3-dihydro- benzoimidazol- 1-yl]-biphenyl- 2-yl}-2-phenyl- acetamide | 1.32 (A) | 543.0 | Ppm 3.65 (s, 2H) 5.60 (s, 2H) 7.06 (d, 1H) 7.15-7.75 (m, 20H) 8.92 (bs, 2H) 9.53 (s, 1H) |
| 5.12 | 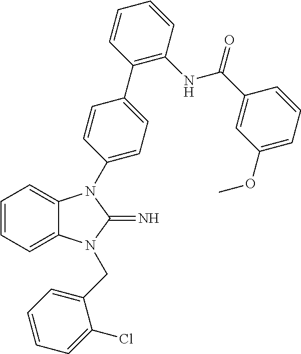 [SMILES: COc1cccc(C(=O)Nc2ccccc2-c2ccc(-n3c(=N)n(Cc4ccccc4Cl)c4ccccc43)cc2)c1] | N-{4′-[3-(2- Chloro-benzyl)- 2-imino- 2,3-dihydro- benzoimidazol- 1-yl]-biphenyl- 2-yl}-3- methoxy- benzamide | 1.36 (A) | 559.1 | |
| 5.13 | 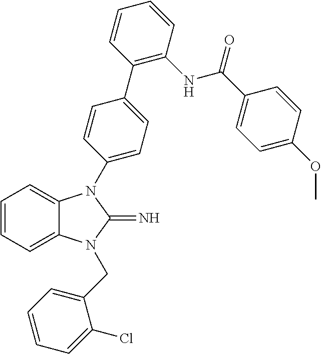 [SMILES: COc1ccc(C(=O)Nc2ccccc2-c2ccc(-n3c(=N)n(Cc4ccccc4Cl)c4ccccc43)cc2)cc1] | N-{4′-[3-(2- Chloro-benzyl)- 2-imino- 2,3-dihydro- benzoimidazol- 1-yl]-biphenyl- 2-yl}-4- methoxy- benzamide | 1.35 (A) | 559.1 | |
| 5.14 | 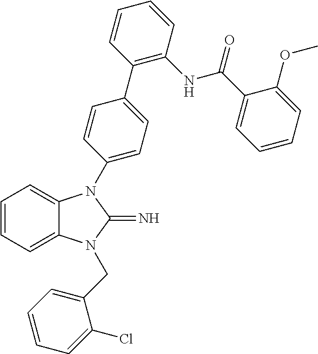 [SMILES: COc1ccccc1C(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}-2- methoxy- benzamide | 1.42 (A) | 559.1 | Ppm 3.70 (s, 3H) 5.59 (s, 2H) 7.03- 7.18 (m, 4H) 7.31- 7.65 (m, 11H) 7.89 (m, 4H) 8.21 (d, 1H) 8.96 (bs, 2H) 9.94 (s, 1H) |
| 5.15 | 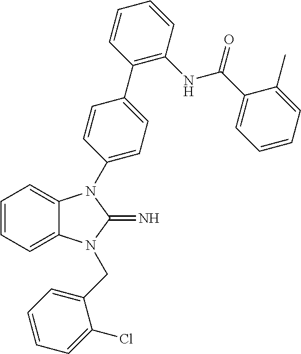 [SMILES: Cc1ccccc1C(=O)Nc1ccccc1-c1ccc(-n2c(=N)n(Cc3ccccc3Cl)c3ccccc32)cc1] | N-{4′-[3-(2- Chloro-benzyl)- 2-imino- 2,3-dihydro- benzoimidazol- 1-yl]-biphenyl- 2-yl}-2-methyl- benzamide | 1.38 (A) | 543.0 | |
| 5.16 | 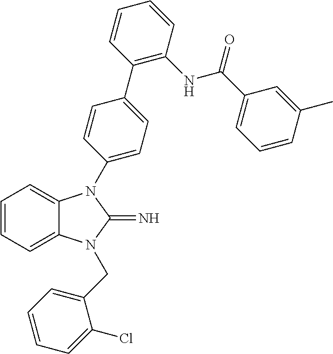 [SMILES: Cc1cccc(C(=O)Nc2ccccc2-c2ccc(-n3c(=N)n(Cc4ccccc4Cl)c4ccccc43)cc2)c1] | N-{4′-[3-(2- Chloro-benzyl)- 2-imino- 2,3-dihydro- benzoimidazol- 1-yl]-biphenyl- 2-yl}-3-methyl- benzamide | 1.39 (A) | 543.0 | |
| 5.17 | 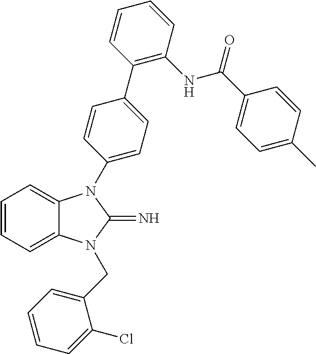 [SMILES: Cc1ccc(C(=O)Nc2ccccc2-c2ccc(-n3c(=N)n(Cc4ccccc4Cl)c4ccccc43)cc2)cc1] | N-{4′-[3-(2- Chloro-benzyl)- 2-imino- 2,3-dihydro- benzoimidazol- 1-yl]-biphenyl- 2-yl}-4-methyl- benzamide | 1.39 (A) | 543.1 | Ppm 2.30 (s, 3H) 5.56 (s, 2H) 6.71 (d, 1H) 7.13 (d, 1H) 7.20-7.65 (m, 12H) 7.77 (m, 6H) 8.94 (s, 2H) 9.91 (s, 1H) |
| 5.18 | 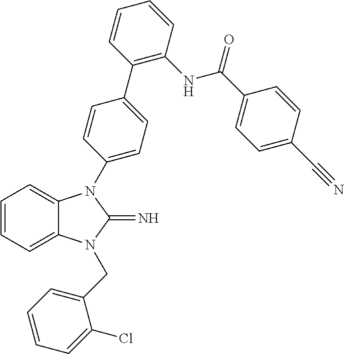 [SMILES: N#Cc1ccc(C(=O)Nc2ccccc2-c2ccc(-n3c(=N)n(Cc4ccccc4Cl)c4ccccc43)cc2)cc1] | N-{4′-[3-(2- Chloro-benzyl)- 2-imino- 2,3-dihydro- benzoimidazol- 1-yl]-biphenyl- 2-yl}-4-cyano- benzamide | 1.33 (A) | 554.0 | |
| 5.19 | 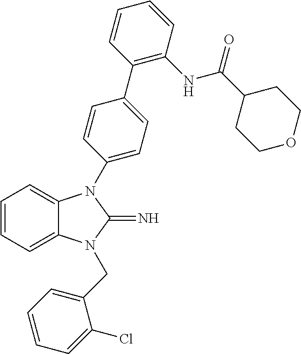 [SMILES: N=c1n(Cc2ccccc2Cl)c2ccccc2n1-c1ccc(-c2ccccc2NC(=O)C2CCOCC2)cc1] | Tetrahydro- pyran-4- carboxylic acid {4′-[3-(2-chloro- benzyl)-2- imino-2,3- dihydro- benzoimidazol- 1-yl]-biphenyl- 2-yl}-amide | 1.24 (A) | 537.1 | |
| 5.20 | 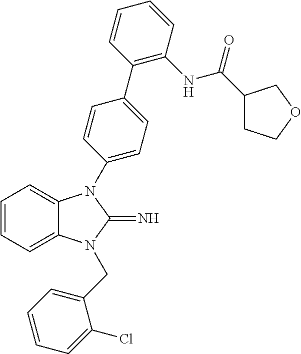 [SMILES: N=c1n(Cc2ccccc2Cl)c2ccccc2n1-c1ccc(-c2ccccc2NC(=O)C2CCOC2)cc1] | Tetrahydro- furan-3- carboxylic acid {4′-[3-(2-chloro- benzyl)-2- imino-2,3- dihydro- benzoimidazol- 1-yl]-biphenyl- 2-yl}-amide | 1.27 (A) | 523.0 | |

*t_{R} [min] (method); **M + H (or specified); ***d⁶-dmso

### Example 6

### N-{4′-[3-(2-Chloro-benzyl)-2-imino-2,3-dihydro-benzoimidazol-1-yl]-4-methylbiphenyl-2-yl}-acetamide

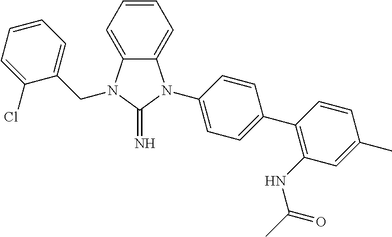 [SMILES: CC(=O)Nc1cc(C)ccc1-c1ccc(-n2c(=N)n(Cc3ccccc3Cl)c3ccccc32)cc1]

To a solution of 4′-[3-(2-Chloro-benzyl)-2-imino-2,3-dihydro-benzoimidazol-1-yl]-4-methyl-biphenyl-2-ylamine (step 1, 33.0 mg, 0.059 mmol) and pyridine (10.6 μL, 0.148 mmol) in CH2Cl2 (500 μL) was added acetyl chloride (5.3 μL, 0.074 mmol) and the resulting mixture stirred at rt for one hour. The medium was then diluted with CH2Cl2 (5 mL), washed with 0.1 N aqueous sodium hydroxide solution (3×5 mL), brine (5 mL), dried over Na2SO4 and finally evaporated to dryness under reduced pressure. The crude product was purified by reverse-phase preparative HPLC (Method E). Product-containing fractions were pooled and evaporated to dryness to furnish the title compound as a TFA salt, 4.0 mg (6%). HPLC/MS (Method A) t_{R} 1.31 minute, M+H 481.0.

### Step 1: 4′-[3-(2-Chloro-benzyl)-2-imino-2,3-dihydro-benzoimidazol-1-yl]-4-methyl-biphenyl-2-ylamine

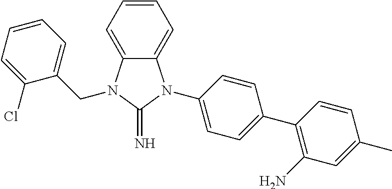 [SMILES: Cc1ccc(-c2ccc(-n3c(=N)n(Cc4ccccc4Cl)c4ccccc43)cc2)c(N)c1]

Intermediate M (100 mg, 0.241 mmol), 2-Amino-4-Methylphenylboronic acid pinacol ester (Boron Molecular BM367) (63.1 mg, 0.265 mmol) and Pd(Ph3P)₄ (14.2 mg, 12.1 μmol) were suspended in DME (1.2 mL) and 2M aqueous Na2CO3 solution (0.6 mL, 1.20 mmol). The resulting reaction mixture was stirred for 17 minutes at 150° C. under microwave irradiation. The reaction mixture was then diluted with MeOH (2 mL) and filtered through a pad of diatomaceous earth which was rinsed with EtOAc (2×4 mL). The resulting organic filtrate was evaporated to dryness and the crude product purified by reverse-phase preparative HPLC (Method E). Product-containing fractions were pooled and evaporated to dryness to furnish the title compound as a TFA salt, 33 mg (25%). HPLC/MS (Method A) t_{R} 1.40 minute, M+H 339.

### Example 7

### 2-Amino-N-{4′-[3-(2-chloro-benzyl)-2-imino-2,3-dihydro-benzoimidazol-1-yl]-biphenyl-2-yl}-acetamide

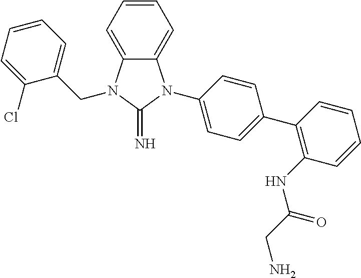 [SMILES: N=c1n(Cc2ccccc2Cl)c2ccccc2n1-c1ccc(-c2ccccc2NC(=O)CN)cc1]

4′-[3-(2-Chloro-benzyl)-2-imino-2,3-dihydro-benzoimidazol-1-yl]-biphenyl-2-ylamine (example 4, 30.0 mg, 0.057 mmol) was treated with a preformed solution of tert-butoxycarbonylamino-acetic acid (11.2 mg, 0.063 mmol), HATU (24.3 mg, 0.063 mmol) and DIPEA (14.9 μL, 0.085 mmol) in NMP (570 μL) for 5 hours at 60° C. The medium was then diluted with CH2Cl2 (5 mL), washed with 0.1 N aqueous sodium hydroxide solution (2×5 mL), brine (5 mL), dried over Na2SO4 and finally evaporated to dryness under reduced pressure. The crude product was treated with a 20% solution of TFA in CH2Cl2 (1.0 ml) for 7 hours at rt. The reaction mixture was then evaporated to dryness. The residue was partitioned between CH2Cl2 (5 mL) and saturated aqueous sodium bicarbonate solution (5 mL). The organic phase was then evaporated to dryness and submitted to purification by reverse-phase preparative HPLC (Method E). Product-containing fractions were pooled and evaporated to dryness to furnish the title compound as a TFA salt, 11.0 mg (32%). HPLC/MS (Method A) t_{R} 0.97 min, [(M+2H)/2] 241.5. 1H NMR (dmso-d6) Ppm 3.66 (bd, 2H) 5.60 (s, 2H) 7.17 (m, 2H) 7.35 (m, 3H) 7.47 (m, 5H) 7.63 (m, 2H) 7.76 (d, 2H) 7.83 (m, 2H) 8.07 (bs, 3H) 8.92 (bs, 2H) 9.85 (s, 1H).

The following examples 7.1 to 7.6 were synthesized in a manner analogous to that used for the synthesis of ex. 7, from ex. 4 and various suitably protected amino acids.

| Ex. | structure | name | HPLC* | MS** | 1H NMR*** |
|---|---|---|---|---|---|
| 7.1 | 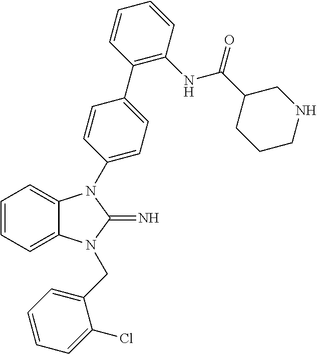 [SMILES: N=c1n(Cc2ccccc2Cl)c2ccccc2n1-c1ccc(-c2ccccc2NC(=O)C2CCCNC2)cc1] | Rac-Piperidine-3- carboxylic acid {4′-[3- (2-chloro-benzyl)-2- imino-2,3-dihydro- benzoimidazol-1-yl]- biphenyl-2-yl}-amide | 1.02 (A) | 536.1 268.6 (M + 2H)/ 2 | |
| 7.2 | 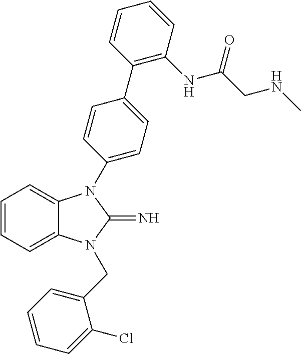 [SMILES: CNCC(=O)Nc1ccccc1-c1ccc(-n2c(=N)n(Cc3ccccc3Cl)c3ccccc32)cc1] | N-{4′-[3-(2-Chloro- benzyl)-2-imino- 2,3-dihydro- benzoimidazol-1-yl]- biphenyl-2-yl}-2- methylamino- acetamide | 1.00 (A) | 248.5 (M + 2H)/ 2 | Ppm 2.45 (s, 3H) 3.82 (bs, 2H) 5.60 (s, 2H) 7.14 (m, 2H) 7.30-7.55 (m, 8H) 7.61 (t, 2H) 7.78 (dd, 4H) 8.81 (bs, 2H) 8.94 (bs, 2H) 10.04 (s, 1H) |
| 7.3 | 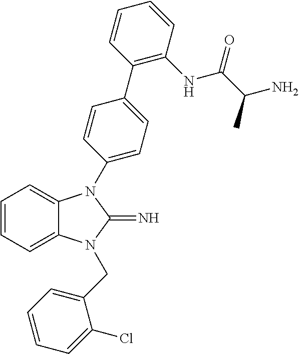 [SMILES: C[C@H](N)C(=O)Nc1ccccc1-c1ccc(-n2c(=N)n(Cc3ccccc3Cl)c3ccccc32)cc1] | (S)-2-Amino-N-{4′-[3- (2-chloro-benzyl)- 2-imino-2,3- dihydro-benzoimidazol- 1-yl]-biphenyl-2-yl}- propionamide | 0.99 (A) | 248.5 (M + 2H)/ 2 | |
| 7.4 | 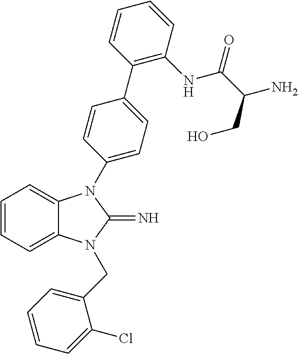 [SMILES: N=c1n(Cc2ccccc2Cl)c2ccccc2n1-c1ccc(-c2ccccc2NC(=O)[C@@H](N)CO)cc1] | (S)-2-Amino-N-{4′-[3- (2-chloro-benzyl)- 2-imino-2,3- dihydro-benzoimidazol- 1-yl]-biphenyl-2-yl}- 3-hydroxy- propionamide | 0.95 (A) | 256.5 (M + 2H)/ 2 | |
| 7.5 | 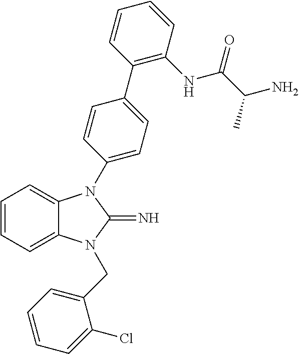 [SMILES: C[C@@H](N)C(=O)Nc1ccccc1-c1ccc(-n2c(=N)n(Cc3ccccc3Cl)c3ccccc32)cc1] | (R)-2-Amino-N-{4′-[3- (2-chloro-benzyl)- 2-imino-2,3- dihydro-benzoimidazol- 1-yl]-biphenyl-2-yl}- propionamide | 0.99 (A) | 248.5 (M + 2H)/ 2 | |
| 7.6 | 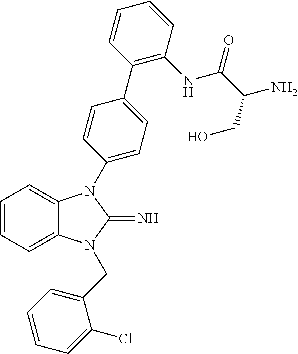 [SMILES: N=c1n(Cc2ccccc2Cl)c2ccccc2n1-c1ccc(-c2ccccc2NC(=O)[C@H](N)CO)cc1] | (R)-2-Amino-N-{4′-[3- (2-chloro-benzyl)- 2-imino-2,3- dihydro-benzoimidazol- 1-yl]-biphenyl-2-yl}- 3-hydroxy- propionamide | 0.95 (A) | 256.5 (M + 2H)/ 2 | |

*t_{R} [min] (method); **M + H (or specified); ***d⁶-dmso

### Example 8

### N-{4′-[3-(2-Chloro-benzyl)-2-imino-2,3-dihydro-benzoimidazol-1-yl]-5-methyl-biphenyl-2-yl}-acetamide

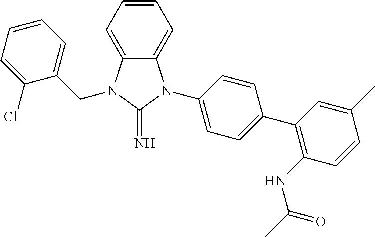 [SMILES: CC(=O)Nc1ccc(C)cc1-c1ccc(-n2c(=N)n(Cc3ccccc3Cl)c3ccccc32)cc1]

The title compound N-{4′-[3-(2-Chloro-benzyl)-2-imino-2,3-dihydro-benzoimidazol-1-yl]-5-methyl-biphenyl-2-yl}-acetamide was synthesized in two steps from intermediate M in a manner analogous to that used for the synthesis of example 6, using 2-amino-3-methylphenylboronic acid pinacol ester instead of 2-amino-4-methylphenylboronic acid pinacol ester in the first synthetic step. HPLC/MS (method A) t_{R} 1.30 min, M+H 481.0. 1H NMR (dmso-d6) Ppm 1.93 (s, 3H) 2.38 (s, 3H) 5.59 (s, 2H) 7.10 (d, 1H) 7.17 (d 1H) 7.25 (d, 2H) 7.30-7.45 (m, 6H) 7.62 (d, 1H) 7.73 (d, 2H) 7.81 (d, 2H) 8.93 (bs, 2H) 9.26 (s, 1H).

### Example 9

### Cyclohexanecarboxylic acid {4′-[3-(2-chloro-benzyl)-2-imino-2,3-dihydro-benzoimidazol-1-yl]-5-methyl-biphenyl-2-yl}-amide

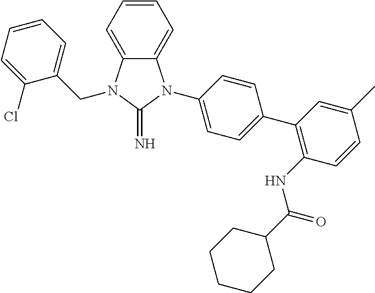 [SMILES: Cc1ccc(NC(=O)C2CCCCC2)c(-c2ccc(-n3c(=N)n(Cc4ccccc4Cl)c4ccccc43)cc2)c1]

The title compound cyclohexanecarboxylic acid {4′-[3-(2-chloro-benzyl)-2-imino-2,3-dihydro-benzoimidazol-1-yl]-5-methyl-biphenyl-2-yl}-amide, TFA salt, was synthesized in two steps from intermediate M in a manner analogous to that used for the synthesis of example 6, using 2-amino-3-methylphenylboronic acid pinacol ester instead of 2-amino-4-methylphenylboronic acid pinacol ester in the first synthetic step and cyclohexyl carbonyl chloride instead of acetyl chloride in the second step. HPLC/MS (method A) t_{R} 1.50 min, M+H 549.1. ¹H NMR (dmso-d6) Ppm 1.22 (m, 6H) 1.64 (m, 4H) 2.21 (t, 1H) 2.38 (s, 3H) 5.58 (s, 2H) 6.97 (s, 1H) 7.15-7.35 (m, 7H) 7.43 (m, 2H) 7.62 (d, 1H) 7.68 (d, 2H) 7.79 (d, 2H) 8.93 (bs, 2H) 9.20 (s, 1H).

### Example 10

### 4′-[3-(2-Chloro-benzyl)-2-imino-2,3-dihydro-benzoimidazol-1-yl]-5-fluorobiphenyl-2-ylamine

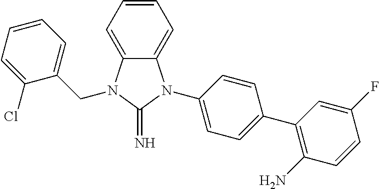 [SMILES: N=c1n(Cc2ccccc2Cl)c2ccccc2n1-c1ccc(-c2cc(F)ccc2N)cc1]

Intermediate M (150 mg, 0.362 mmol), 2-amino-5-fluorophenylboronic acid (63 mg, 0.398 mmol) and Pd(Ph3P)₄ (21 mg, 18 μmol) were suspended in DME (1.8 mL) and 2M aqueous Na2CO3 solution (0.9 mL, 1.8 mmol). The resulting reaction mixture was stirred for 17 minutes at 150° C. under microwave irradiation. The reaction mixture was then diluted with MeOH (40 mL) and filtered through a pad of diatomaceous earth which was rinsed with EtOAc (2×30 mL). The resulting organic filtrate was evaporated to dryness, the residue was taken up in CH2Cl2 (30 mL) and 0.1 N aqueous sodium hydroxide solution (25 mL). The phases were separated and the aqueous portion was back-extracted with CH2Cl2 (25 mL). The combined organic layers were washed with brine, dried over Na2SO4 and evaporated to give the title compound as a grey solid. No further purification was carried out. HPLC/MS (method A) t_{R} 1.36 min, M+H 443.0, (M+2H)/2 222.0. ¹H NMR (dmso-d6) Ppm 4.85 (bs, 2H) 5.21 (s, 2H) 6.75-7.00 (m, 8H) 7.10 (bs, 1H) 7.32 (m, 2H) 7.50-7.75 (m, 8H).

### Example 11

### N-{4′-[3-(2-Chloro-benzyl)-2-imino-2,3-dihydro-benzoimidazol-1-yl]-5-fluoro-biphenyl-2-yl}-acetamide

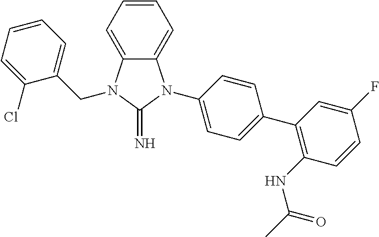 [SMILES: CC(=O)Nc1ccc(F)cc1-c1ccc(-n2c(=N)n(Cc3ccccc3Cl)c3ccccc32)cc1]

The title compound N-{4′-[3-(2-Chloro-benzyl)-2-imino-2,3-dihydro-benzoimidazol-1-yl]-5-fluoro-biphenyl-2-yl}-acetamide, TFA salt, was synthesized in one step from example 10 in a manner analogous to that used for the synthesis of example 5, using acetyl chloride instead of 3-cyanobenzoyl chloride. HPLC/MS (method A) t_{R} 1.19 minute, M+H 485.0. 1H NMR (dmso-d6) ppm 1.94 (s, 3H) 5.59 (s, 2H) 7.11 (d, 1H) 7.28 (d, 1H) 725-7.45 (m, 7H) 7.55 (m, 1H) 7.62 (d, 1H) 7.78 (d, 2H) 7.84 (d, 2H) 8.94 (bs, 2H) 9.37 (s, 1H).

### Example 12

### 1-{4′-[3-(2-Chloro-benzyl)-2-imino-2,3-dihydro-benzoimidazol-1-yl]-biphenyl-2-yl}-3-methyl-urea

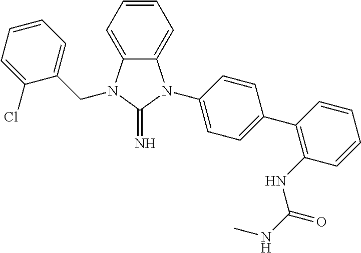 [SMILES: CNC(=O)Nc1ccccc1-c1ccc(-n2c(=N)n(Cc3ccccc3Cl)c3ccccc32)cc1]

To a solution of intermediate Q (50 mg, 0.095 mmol) in CH2Cl2 were added pyridine (27.6 μL, 0.342 mmol) and methyl isocyanate (80.3 μL, 1.36 mmol) and the resulting solution stirred 24 hours at rt. The reaction mixture was then diluted with CH2Cl2 (5 mL) and washed successively with 1N aqueous sodium hydroxide solution (3×5 mL) and brine (5 mL). The organic phase was dried over Na2SO4 and evaporated to dryness. The residue was treated with a 3/7 mixture of TFA in CH2Cl2 (1 mL) for one hour at rt. The reaction mixture was then diluted with CH2Cl2 (5 mL) and washed with 1N aqueous sodium hydroxide solution (3×5 mL) and brine (5 mL). The organic phase was dried over Na2SO4 and evaporated to dryness. The residue was purified by reverse phase preparative HPLC (method E) to afford the title compound as a TFA salt, 11 mg (19%). HPLC/MS (method A) t_{R} 1.18 minute, M+H 482.1. ¹H NMR (dmso-d6) ppm 2.65 (d, 3H) 5.59 (s, 2H) 6.35 (q, 1H) 7.10-7.95 (m, 17H) 8.91 (bs, 2H).

### Example 13

### 1-{4′-[3-(2-Chloro-benzyl)-2-imino-2,3-dihydro-benzoimidazol-1-yl]-biphenyl-2-yl}-3-ethyl-urea

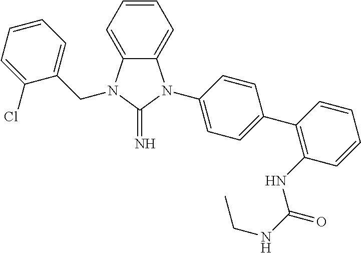 [SMILES: CCNC(=O)Nc1ccccc1-c1ccc(-n2c(=N)n(Cc3ccccc3Cl)c3ccccc32)cc1]

The title compound 1-{4′-[3-(2-Chloro-benzyl)-2-imino-2,3-dihydro-benzoimidazol-1-yl]-biphenyl-2-yl}-3-ethyl-urea, TFA salt, was synthesized in one step from intermediate Q in a manner analogous to that used for the synthesis of example 12, using ethyl isocyanate instead of methyl isocyanate. HPLC/MS (method A) t_{R} 1.23 min., M+H 496.1; 1H NMR (dmso-d6) ppm 1.02 (t, 3H) 3.08 (q, 2H) 5.59 (s, 2H) 6.45 (t, 1H) 7.15-7.95 (m, 17H) 8.94 (bs, 2H).

### Example 14

### 5-Chloro-4′-[3-(2-chloro-benzyl)-2-imino-2,3-dihydro-benzoimidazol-1-yl]-biphenyl-2-carboxylic acid methylamide

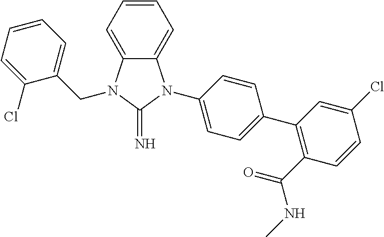 [SMILES: CNC(=O)c1ccc(Cl)cc1-c1ccc(-n2c(=N)n(Cc3ccccc3Cl)c3ccccc32)cc1]

To a solution of 4′-[2-[(E)-tert-Butoxycarbonylimino]-3-(2-chloro-benzyl)-2,3-dihydro-benzoimidazol-1-yl]-5-chloro-biphenyl-2-carboxylic acid (step 1, 120 mg, 0.204 mmol) in DMF (2.0 mL) were added HATU (85 mg, 0.224 mmol) and DIPEA (53.4 μL, 0.306 mmol). The resulting yellow solution was stirred for 5 minutes at rt prior to the addition of a solution of methylamine 2M in THF (510 μL, 1.020 mmol). The mixture was stirred further at 60° C. for one hour before the reaction was quenched by the addition of 10% aqueous citric acid solution (15 mL). The phases were separated and the aqueous layer was extracted with EtOAc (3×10 mL). The combined organic phases were washed successively with 10% aqueous citric acid solution (3×10 mL) and 1M aqueous sodium hydroxide solution (2×10 mL), dried over Na2SO4 and evaporated to dryness. The residue was taken up in CH2CL2 (1.4 mL) and treated with TFA (0.6 mL) for one hour at rt. The mixture was again evaporated to dryness and the residue partitioned between EtOAc (5 mL) and saturated aqueous sodium bicarbonate solution (5 mL). The organic phase was dried over Na2SO4 and concentrated. The crude product was purified by reverse phase preparative HPLC (method E). Product-containing fractions were lyophilized and the product salt was desalted by partitioning between CH2Cl2 and saturated aqueous sodium bicarbonate solution, to finally afford the title compound as a white solid, 40 mg (39%). HPLC/MS (method A) t_{R} 1.31 minute, M+H 501.0. 1H NMR (Dimethylsulfoxide-d6) ppm 2.62 (d, 3H) 5.23 (s, 2H) 6.80-7.70 (m, 15H) 8.21 (m, 1H).

### Step 1: 4′-[2-[(E)-tert-Butoxycarbonylimino]-3-(2-chloro-benzyl)-2,3-dihydro-benzoimidazol-1-yl]-5-chloro-biphenyl-2-carboxylic acid

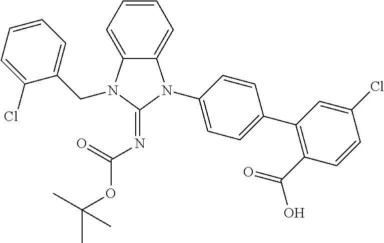 [SMILES: CC(C)(C)OC(=O)/N=c1\n(Cc2ccccc2Cl)c2ccccc2n1-c1ccc(-c2cc(Cl)ccc2C(=O)O)cc1]

The title compound 4′-[2-[(E)-tert-Butoxycarbonylimino]-3-(2-chloro-benzyl)-2,3-dihydro-benzoimidazol-1-yl]-5-chloro-biphenyl-2-carboxylic acid was synthesized from intermediate P and 2-Carboxy-5-Chlorophenylboronic acid in a manner analogous to that used for the synthesis of intermediate Q, heating for 26 hours at 90° C. HPLC/MS (method A) t_{R} 1.75 minute, M+H 588.0. 1H NMR (Dimethylsulfoxide-d6) ppm 1.08 (s, 9H) 5.39 (s, 2H) 7.01-7.82 (m, 15H).

### Example 15

### N-{5-Acetyl-4′-[3-(2-chloro-benzyl)-2-imino-2,3-dihydro-benzoimidazol-1-yl]-biphenyl-2-yl}-acetamide

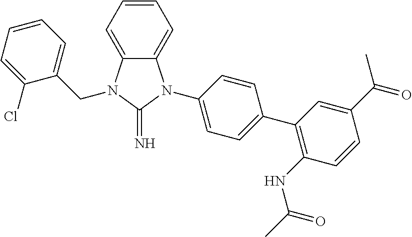 [SMILES: CC(=O)Nc1ccc(C(C)=O)cc1-c1ccc(-n2c(=N)n(Cc3ccccc3Cl)c3ccccc32)cc1]

N-(4-Acetyl-2-bromo-phenyl)-acetamide (186 mg, 0.727 mmol), bis(pinacolato)diboron (240 mg, 0.945 mmol), Pd(PPh3)2Cl2 (25.5 mg, 0.036 mmol) and potassium acetate (214 mg, 2.181 mmol) as a suspension in dioxane (7.27 mL) in a schlenk tube under Argon were stirred at 80° C. for 150 minutes. The reaction was then cooled and filtered through a pad of celite. The pad was washed with EtOAc (3×5 mL) and the filtrate was evaporated to dryness. The residue was dissolved in DME (2.4 ml) and intermediate M (200 mg, 0.485 mmol), Pd(PPh3)4 (28.0 mg, 0.024 mmol), Na2CO3 (1.212 ml, 2.423 mmol) were added. The resulting reaction mixture was stirred for 17 minutes at 150° C. under microwave irradiation. The medium was then filtered again through a pad of celite which was washed with EtOAc (3×10 mL). The filtrate was evaporated to dryness and purified by chromatography on amine-functionalized silica (Biotage™ 25+S KP-NH column), eluting with a 0% to 100% gradient of eluent B (EtOAc) in eluent A (heptane/CH₂Cl₂:1/1). Product containing fractions were pooled and evaporated to yield the title compound as a brown solid, 82 mg (30%). HPLC/MS (method A) t_{R} 1.14 min, M+H 509.1.

### Example 16

### N-{4′-[3-(2-Chloro-benzyl)-2-imino-2,3-dihydro-benzoimidazol-1-yl]-5-trifluoromethoxy-biphenyl-2-yl}-acetamide

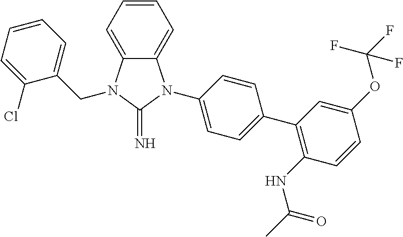 [SMILES: CC(=O)Nc1ccc(OC(F)(F)F)cc1-c1ccc(-n2c(=N)n(Cc3ccccc3Cl)c3ccccc32)cc1]

The title compound N-{4′-[3-(2-Chloro-benzyl)-2-imino-2,3-dihydro-benzoimidazol-1-yl]-5-trifluoromethoxy-biphenyl-2-yl}-acetamide was synthesized from intermediate M and N-(2-Bromo-4-trifluoromethoxy-phenyl)-acetamide in a manner analogous to that used for the synthesis of example 15. HPLC/MS (method A) t_{R} 1.53 min, M+H 551.1. 1H NMR (Dimethylsulfoxide-d6) Ppm 1.95 (s, 3H), 5.3 (s, 2H) 6.95-7.80 (m, 15H) 9.5 (s, 1H).

### Example 17

### N-{4′-[3-(2-Chloro-benzyl)-2-imino-2,3-dihydro-benzoimidazol-1-yl]-5-trifluoromethyl-biphenyl-2-yl}-acetamide

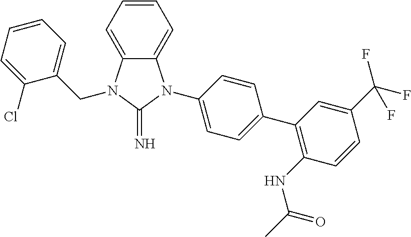 [SMILES: CC(=O)Nc1ccc(C(F)(F)F)cc1-c1ccc(-n2c(=N)n(Cc3ccccc3Cl)c3ccccc32)cc1]

The title compound N-{4′-[3-(2-Chloro-benzyl)-2-imino-2,3-dihydro-benzoimidazol-1-yl]-5-trifluoromethyl-biphenyl-2-yl}-acetamide was synthesized from intermediate M and N-(2-Bromo-4-trifluoromethyl-phenyl)-acetamide in a manner analogous to that used for the synthesis of example 15. HPLC/MS (method A) t_{R} 1.55 min, M+H 535.0. 1H NMR (dmso-d6) Ppm 2.01 (s, 3H) 5.59 (s, 2H) 7.17 (m, 2H) 7.39 (m, 5H) 7.62 (m, 2H) 7.84 (m, 5H) 7.98 (d, 1H) 8.90 (s, 2H) 9.49 (s, 1H).

### Example 18

### N-{5-tert-Butyl-4′-[3-(2-chloro-benzyl)-2-imino-2,3-dihydro-benzoimidazol-1-yl]-biphenyl-2-yl}-acetamide

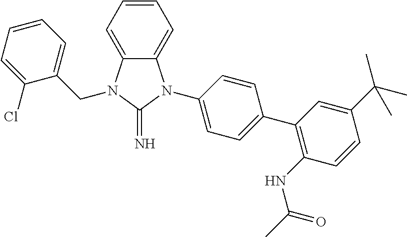 [SMILES: CC(=O)Nc1ccc(C(C)(C)C)cc1-c1ccc(-n2c(=N)n(Cc3ccccc3Cl)c3ccccc32)cc1]

The title compound N-{5-tert-Butyl-4′-[3-(2-chloro-benzyl)-2-imino-2,3-dihydro-benzoimidazol-1-yl]biphenyl-2-yl}-acetamide was synthesized from intermediate M and N-(2-Bromo-4-tert-butyl-phenyl)-acetamide in a manner analogous to that used for the synthesis of example 15. HPLC/MS (method A) t_{R} 1.40 minute, M+H 523.1.

### Example 19

### N-[4′-[3-(2-Chloro-benzyl)-2-imino-2,3-dihydro-benzoimidazol-1-yl]-5-(1-hydroxy-ethyl)-biphenyl-2-yl]-acetamide

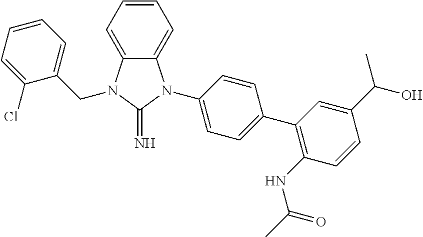 [SMILES: CC(=O)Nc1ccc(C(C)O)cc1-c1ccc(-n2c(=N)n(Cc3ccccc3Cl)c3ccccc32)cc1]

The title compound N-[4′-[3-(2-Chloro-benzyl)-2-imino-2,3-dihydro-benzoimidazol-1-yl]-5-(1-hydroxy-ethyl)-biphenyl-2-yl]-acetamide was synthesized from intermediate M and N-[2-Bromo-4-(1-hydroxy-ethyl)-phenyl]-acetamide (intermediate R) in a manner analogous to that used for the synthesis of example 15. HPLC/MS (method A) t_{R} 0.99 min, M+H 511.0.

### Example 20

### N-[4′-[3-(2-Chloro-benzyl)-2-imino-2,3-dihydro-benzoimidazol-1-yl]-5-(1-hydroxy-1-methyl-ethyl)-biphenyl-2-yl]-acetamide

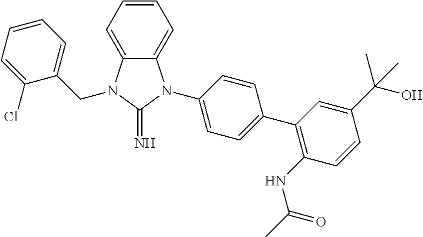 [SMILES: CC(=O)Nc1ccc(C(C)(C)O)cc1-c1ccc(-n2c(=N)n(Cc3ccccc3Cl)c3ccccc32)cc1]

To a solution of N-{5-Acetyl-4′-[3-(2-chloro-benzyl)-2-imino-2,3-dihydro-benzoimidazol-1-yl]-biphenyl-2-yl}-acetamide (example 15, 82 mg, 0.145 mmol) in THF (1450 μL) under Ar was added MeMgBr (1.4M solution in toluene/THF 3/1, 311 μL, 0.435 mmol) dropwise. The resulting reaction mixture was stirred for one hour at rt, resulting in a roughly 30% HPLC UV conversion. The reaction was quenched with water (2 mL) and then diluted with CH2Cl2 (5 mL). The phases were separated and the organic layer was dried over Na2SO4 and concentrated to a dark residue. The crude product was purified reverse-phase preparative HPLC (Method E, gradient from 10% MeCN (+0.1% TFA) in water (+0.1% TFA) to 70% over 14 minutes) yielding, after pooling and lyophilisation of product-containing fractions, to the pre title compound as its TFA salt, 8 mg (8%). HPLC/MS (Method D) t_{R} 1.93 minute, M+H 524.8. 1H NMR (Dimethylsulfoxide-d6) Ppm 1.64 (s, 6H) 2.03 (s, 3H) 5.70 (s, 2H) 7.10-7.90 (m, 15H) 8.73 (s, 1H) 8.94 (bs, 2H).

### Example 21

### N-{4′-[3-(2-Chloro-benzyl)-2-imino-2,3-dihydro-benzoimidazol-1-yl]-6-methyl-biphenyl-2-yl}-acetamide

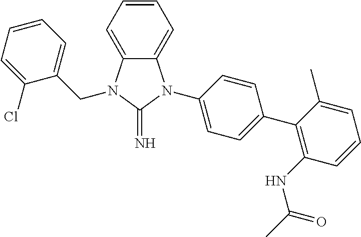 [SMILES: CC(=O)Nc1cccc(C)c1-c1ccc(-n2c(=N)n(Cc3ccccc3Cl)c3ccccc32)cc1]

The title compound N-{4′-[3-(2-Chloro-benzyl)-2-imino-2,3-dihydro-benzoimidazol-1-yl]-6-methyl-biphenyl-2-yl}-acetamide was synthesized from intermediate M and N-(2-Bromo-3-methyl-phenyl)-acetamide in a manner analogous to that used for the synthesis of example 15. HPLC/MS (Method D) t_{R} 2.18 minute, M+H 480.8. 1H NMR (Acetone-d6) Ppm 1.90 (s, 3H) 2.16 (s, 3H) 5.77 (s, 2H) 7.13-7.90 (m, 17H) 9.36 (bs, 2H).

### Example 22

### N-(5-Chloro-3-{4-[3-(2-chloro-benzyl)-2-imino-2,3-dihydro-benzoimidazol-1-yl]-phenyl}-pyridin-2-yl)-acetamide

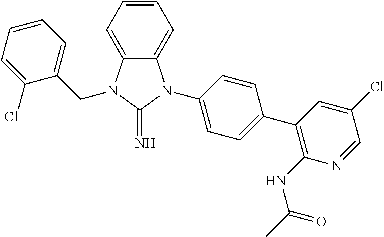 [SMILES: CC(=O)Nc1ncc(Cl)cc1-c1ccc(-n2c(=N)n(Cc3ccccc3Cl)c3ccccc32)cc1]

Intermediate S (35 mg, 0.070 mmol), N-(3-Bromo-5-chloro-pyridin-2-yl)-acetamide (obtained trivially by acetylation of commercially available 3-Bromo-5-chloro-pyridin-2-ylamine by acetyl chloride in CH2CL2 in the presence of pyridine, 25.4 mg, 0.102 mmol) and Pd(Ph3P)4 (5.4 mg, 4.6 μmol) were suspended in DME (463 μL) and 2M aqueous Na2CO3 solution (232 μL, 0.463 mmol). The resulting reaction mixture was stirred for 17 minutes at 150° C. under microwave irradiation. The reaction mixture was then diluted with EtOAc (2 mL) and filtered through a pad of diatomaceous earth which was rinsed with EtOAc (2×2 mL). The resulting organic filtrate was evaporated to dryness and the crude product purified by reverse-phase preparative HPLC (Method E, Gradient from 35% ((MeCN/MeOH 1/3)+0.1% TFA) in (Water+0.1% TFA) to 65% over 14 minutes). Product-containing fractions were pooled and evaporated to dryness. The residue was further purified by preparative thin layer chromatography on silica (20×20 cm plate from Analtech, 500 um thickness), eluting with ethyl acetate+1% NH4OH, to yield the title compound as a solid, 7.3 mg (21%). HPLC/MS (Method F) t_{R} 2.02 minute, M+H 502.7.

### Example 23

### N-(6-Chloro-2-{4-[3-(2-chloro-benzyl)-2-imino-2,3-dihydro-benzoimidazol-1-yl]-phenyl}-pyridin-3-yl)-acetamide

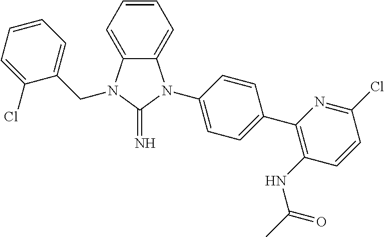 [SMILES: CC(=O)Nc1ccc(Cl)nc1-c1ccc(-n2c(=N)n(Cc3ccccc3Cl)c3ccccc32)cc1]

The title compound N-(6-Chloro-2-{4-[3-(2-chloro-benzyl)-2-imino-2,3-dihydrobenzoimidazol-1-yl]-phenyl}-pyridin-3-yl)-acetamide was synthesized from intermediate S and N-(2-Bromo-6-chloro-pyridin-3-yl)-acetamide (obtained trivially by acetylation of commercially available 2-bromo-6-chloro-pyridin-3-ylamine by acetyl chloride in CH2CL2 in the presence of pyridine) in a manner analogous to that used for the synthesis of example 22, without the need for thin layer chromatography after the reverse-phase prepartitive HPLC purification step. HPLC/MS (Method D) t_{R} 2.08 minute, M+H 502.1. 1H NMR (Dimethylsulfoxide-d6) Ppm 2.03 (s, 3H) 5.23 (s, 2H) 6.86-7.02 (m, 4H) 7.25-7.38 (m, 2H) 7.54 (d, 2H) 7.71 (m, 2H) 7.88 (m, 2H) 8.07 (d, 1H) 9.81 (s, 1H).

### Example 24

### N-{4′-[2-Imino-3-(1H-indol-4-ylmethyl)-2,3-dihydro-benzoimidazol-1-yl]-biphenyl-2-yl}-acetamide

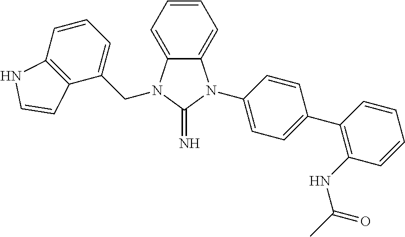 [SMILES: CC(=O)Nc1ccccc1-c1ccc(-n2c(=N)n(Cc3cccc4[nH]ccc34)c3ccccc32)cc1]

A mixture of 4-[3-(4-Bromo-phenyl)-2-imino-2,3-dihydro-benzoimidazol-1-ylmethyl]-indole-1-carboxylic acid tert-butyl ester (intermediate 0, 200 mg, 0.387 mmol), 2-acetamidophenylboronic acid (76 mg, 0.425 mmol) and Pd(Ph3P)₄ (22 mg, 19 μmol) in DME (2.0 mL) and 2M aqueous sodium carbonate solution (0.96 mL, 1.933 mmol) in a sealed pressure vial was stirred for 18 hours at 90° C. The temperature was then raised to 150° C. to effect thermal removal of the Boc protecting group. The reaction mixture was then diluted with MeOH (3 mL) and filtered through a pad of diatomaceous earth which was rinsed with EtOAc (3×10 mL). The resulting organic filtrate was evaporated to dryness. The residue was taken up in EtOAc (50 mL) and washed with 0.1 N aqueous sodium hydroxide solution (2×25 mL) and brine (25 mL). The organic layer was then dried over Na2SO4 and evaporated to a crude solid. The crude product was purified by reverse-phase preparative HPLC (Method E, Gradient from 20% (MeCN+0.1% TFA) in (Water+0.1% TFA) to 80% over 14 minutes). Product-containing fractions were pooled and evaporated to dryness. The residual TFA salt was partitioned between CH2Cl2 and saturated aqueous sodium bicarbonate solution to finally obtain the title product as a free base, 47 mg (26%). HPLC/MS (Method A) t_{R} 1.15 minute, M+H 472.1. 1H NMR (Dimethylsulfoxide-d6) Ppm 1.94 (s., 3H) 5.38 (s., 2H) 6.84 (m., 5H) 7.02 (m., 3H) 7.38 (m., 5H) 7.60 (m., 5H) 9.39 (s., 1H) 11.19 (s., 1H).

### Example 25

### N-{5-Chloro-4′-[2-imino-3-(1H-indol-4-ylmethyl)-2,3-dihydro-benzoimidazol-1-yl]-biphenyl-2-yl}-acetamide

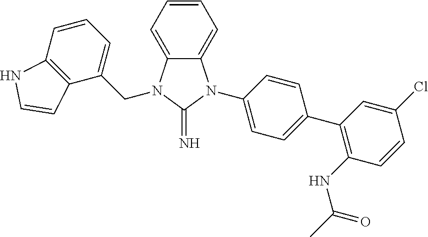 [SMILES: CC(=O)Nc1ccc(Cl)cc1-c1ccc(-n2c(=N)n(Cc3cccc4[nH]ccc34)c3ccccc32)cc1]

A mixture of 4-[3-(4-Bromo-phenyl)-2-imino-2,3-dihydro-benzoimidazol-1-ylmethyl]-indole-1-carboxylic acid tert-butyl ester (intermediate 0, 200 mg, 0.387 mmol), 2-acetamido-5-chlorophenylboronic acid (91 mg, 0.425 mmol) and Pd(Ph3P)₄ (22 mg, 19 μmol) in DME (2.0 mL) and 2M aqueous sodium carbonate solution (0.96 mL, 1.933 mmol) in a sealed pressure vial was stirred for 18 hours at 90° C. After this time, 2-acetamido-5-chlorophenylboronic acid (45 mg, 0.213 mmol) and Pd(Ph3P)4 (22 mg, 19 μmol) were added and the temperature was raised to 150° C. to effect total conversion of the starting material to product and the thermal removal of the Boc protecting group. The reaction mixture was then diluted with MeOH (3 mL) and filtered through a pad of diatomaceous earth which was rinsed with EtOAc (3×10 mL). The resulting organic filtrate was evaporated to dryness. The residue was taken up in EtOAc (50 mL) and washed with 0.1 N aqueous sodium hydroxide solution (2×25 mL) and brine (25 mL). The organic layer was then dried over Na2SO4 and evaporated to a crude solid. The crude product was purified by reverse-phase preparative HPLC (Method E, Gradient from 20% (MeCN+0.1% TFA) in (Water+0.1% TFA) to 80% over 14 minutes). Product-containing fractions were pooled and evaporated to dryness. The residual TFA salt was partitioned between CH2Cl2 and saturated aqueous sodium bicarbonate solution to finally obtain the title product as a free base, 41 mg (21%). HPLC/MS (Method A) t_{R} 1.26 minute, M+H 506.1. 1H NMR (Acetone-d6) Ppm 1.94 (s, 3H) 5.49 (s, 2H) 6.91 (m, 5H) 7.11 (m, 2H) 7.40 (m, 4H) 7.73 (m, 4H) 8.05 (d, 1H) 8.70 (s, 1H) 10.40 (s, 1H).

The following ex. 25.1 to 25.6 were synthesized in analogy to the synthesis of ex. 25, from intermediate O and various aryl boronic acids or boronate esters.

| Ex. | structure | name | HPLC* | MS** | 1H NMR*** |
|---|---|---|---|---|---|
| 25.1 | 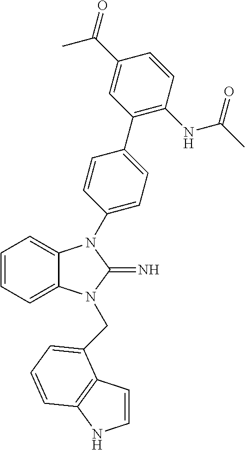 [SMILES: CC(=O)Nc1ccc(C(C)=O)cc1-c1ccc(-n2c(=N)n(Cc3cccc4[nH]ccc34)c3ccccc32)cc1] | N-{5-Acetyl-4′-[2- imino-3-(1H-indol- 4-ylmethyl)-2,3- dihydro-benzoimidazol- 1-yl]-biphenyl- 2-yl}-acetamide | 1.23 (A) | 514.0 | |
| 25.2 | 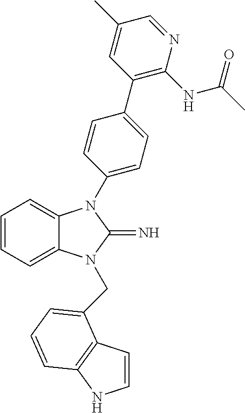 [SMILES: CC(=O)Nc1ncc(C)cc1-c1ccc(-n2c(=N)n(Cc3cccc4[nH]ccc34)c3ccccc32)cc1] | N-(3-{4-[2-Imino-3- (1H-indol-4-ylmethyl)- 2,3-dihydro- benzoimidazol-1-yl]- phenyl}-5-methyl- pyridin-2-yl)- acetamide | 1.17 (A) | 487.1 (60%); (M + 2H)/ 2 244.1 (100%) | |
| 25.3 | 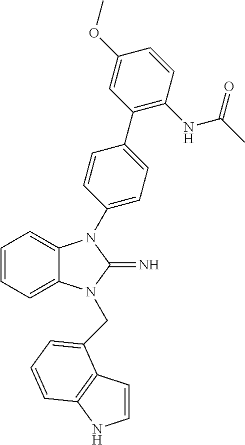 [SMILES: COc1ccc(NC(C)=O)c(-c2ccc(-n3c(=N)n(Cc4cccc5[nH]ccc45)c4ccccc43)cc2)c1] | N-{4′-[2-Imino-3- (1H-indol-4-ylmethyl)- 2,3-dihydro- benzoimidazol-1-yl]- 5-methoxy- biphenyl-2-yl}- acetamide | 1.11 (A) | 502.1 | Ppm 1.91 (s, 3H) 3.82 (s, 3H) 5.38 (s, 2H) 6.75-7.75 (m, 17H) 9.27 (s, 1H) 11.19 (s, 1H) |
| 25.4 | 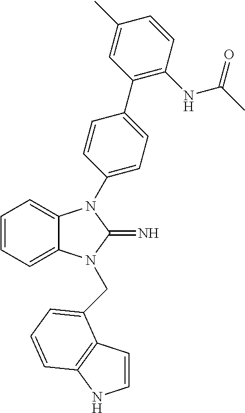 [SMILES: CC(=O)Nc1ccc(C)cc1-c1ccc(-n2c(=N)n(Cc3cccc4[nH]ccc34)c3ccccc32)cc1] | N-{4′-[2-Imino-3- (1H-indol-4-ylmethyl)- 2,3-dihydro- benzoimidazol-1-yl]- 5-methyl-biphenyl- 2-yl}-acetamide | 2.09 (D) | 485.9 | |
| 25.5^{A} | 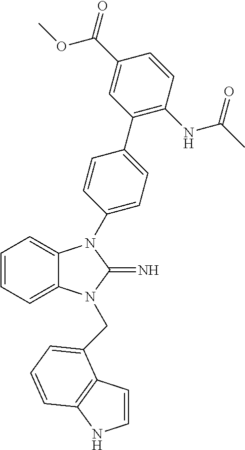 [SMILES: COC(=O)c1ccc(NC(C)=O)c(-c2ccc(-n3c(=N)n(Cc4cccc5[nH]ccc45)c4ccccc43)cc2)c1] | 6-Acetylamino-4′- [2-imino-3-(1H-indol- 4-ylmethyl)-2,3- dihydro-benzoimidazol- 1-yl]- biphenyl-3- carboxylic acid methyl ester | 1.15 (A) | 530.1 | Ppm 2.00 (s, 3H) 3.88 (s, 3H) 5.40 (s, 2H) 6.60-8.00 (m, 16H) 9.52 (s, 1H) 11.20 (s, 1H) |
| 25.6^{B} | 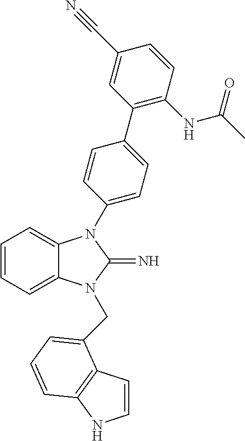 [SMILES: CC(=O)Nc1ccc(C#N)cc1-c1ccc(-n2c(=N)n(Cc3cccc4[nH]ccc34)c3ccccc32)cc1] | N-{5-Cyano-4′-[2- imino-3-(1H-indol- 4-ylmethyl)-2,3- dihydro-enzoimidazol- 1-yl]-biphenyl-2- yl}-acetamide | 1.10 (A) | 497.1 | Ppm 2.09 (s, 3H) 5.48 (s, 2H) 6.90 (m, 5H) 7.10 (m, 2H) 7.38 (m, 2H) 7.79 (m, 6H) 8.45 (d, 1H) 8.82 (s, 1H) 10.38 (s, 1H), recorded in aceton-d6 instead of dimethylsulfoxyde- d6 |

*t_{R} [min] (method); **M + H (or specified); ***d⁶-dmso;
^{A}Synthesized using 4-Acetylamino-3-(4,4,5,5-tetramethyl-[1,3,2]dioxaborolan-2-yl)-benzoic acid methyl ester (intermediate U).
^{B}synthesized using N-[4-Cyano-2-(4,4,5,5-tetramethyl[1,3,2]dioxaborolan-2-yl)-phenyl]-acetamide, obtained from N-(2-Bromo-4-cyano-phenyl)-acetamide in a manner analogous to that used for the synthesis of intermediate U.

### Example 26

### 5-Chloro-4′-[2-imino-3-(1H-indol-4-ylmethyl)-2,3-dihydro-benzoimidazol-1-yl]-biphenyl-2-carboxylic acid methylamide

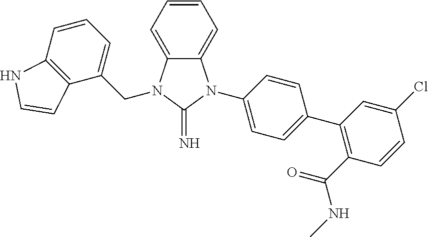 [SMILES: CNC(=O)c1ccc(Cl)cc1-c1ccc(-n2c(=N)n(Cc3cccc4[nH]ccc34)c3ccccc32)cc1]

To a solution of 4-[2-[(E)-tert-Butoxycarbonylimino]-3-(2′-carboxy-5′-chloro-biphenyl-4-yl)-2,3-dihydro-benzoimidazol-1-ylmethyl]-indole-1-carboxylic acid tert-butyl ester (step 1, 248 mg, 0.358 mmol) was added HATU (150 mg, 0.394 mmol) followed by DIPEA (0.094 mL, 0.537 mmol). This mixture was stirred for 5 minutes at rt before the addition of 2M methylamine solution in THF (0.895 mL, 1.790 mmol). The reaction mixture was heated to 60° C. and stirred for one hour. The medium was then diluted with EtOAc (10 mL) and washed successively with 0.1 N aqueous sodium hydroxide solution (3×10 mL), 10% aqueous citric acid solution (2×10 mL) and brine (10 mL). The organic phase was then dried over Na2SO4 and evaporated to dryness. The resulting residue was taken up in CH2Cl2 (2 mL) and treated with TFA (0.7 mL) for 90 minutes at rt. The medium was then diluted with CH2Cl2 (10 mL) and washed with saturated aqueous sodium bicarbonate solution (5 mL). The organic phase was then evaporated to dryness. The crude product was purified by reverse-phase preparative HPLC (Method E, Gradient from 20% (MeCN+0.1% TFA) in (Water+0.1% TFA) to 80% over 14 minutes). Product-containing fractions were pooled and evaporated to dryness. The residual TFA salt was partitioned between CH2Cl2 and saturated aqueous sodium bicarbonate solution to finally obtain the title product as a free base, 28 mg (15%). HPLC/MS (Method A) t_{R} 1.33 minute, M+H 506.0. 1H NMR (Dimethylsulfoxide-d6) Ppm 2.65 (s, 3H) 5.40 (s, 2H) 6.75-7.70 (m, 17H) 8.20 (bd, 1H) 11.19 (s, 1H).

### Step 1: 4-[2-[(E)-tert-Butoxycarbonylimino]-3-(2′-carboxy-5′-chloro-biphenyl-4-yl)-2,3-dihydro-benzoimidazol-1-ylmethyl]-indole-1-carboxylic acid tert-butyl ester

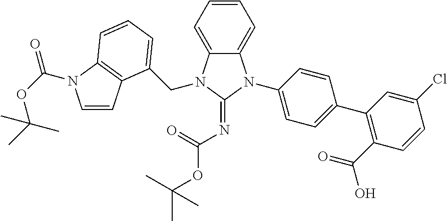 [SMILES: CC(C)(C)OC(=O)/N=c1\n(Cc2cccc3c2ccn3C(=O)OC(C)(C)C)c2ccccc2n1-c1ccc(-c2cc(Cl)ccc2C(=O)O)cc1]

A mixture of 4-{3-(4-Bromo-phenyl)-2-[(E)-tert-butoxycarbonylimino]-2,3-dihydro-benzoimidazol-1-ylmethyl}-indole-1-carboxylic acid tert-butyl ester (intermediate T, 664 mg, 1.075 mmol), 2-carboxy-5-chlorophenylboronic acid (237 mg, 1.183 mmol) and Pd(Ph3P)4 (62.1 mg, 54 μmol) in DME (5.3 mL) and 2M aqueous sodium carbonate solution (2.68 mL, 5.38 mmol) in a sealed pressure vial was stirred for 18 hours at 90° C. The reaction mixture was then diluted with MeOH (5 mL) and filtered through a pad of diatomaceous earth which was rinsed with EtOAc (2×10 mL). The resulting organic filtrate was evaporated to dryness. The residue was taken up in EtOAc (20 mL) and washed with 0.1 N aqueous sodium hydroxide solution (3×10 mL) and brine (10 mL). The organis layer was then dried over Na2SO4 and evaporated to a crude solid. The resulting crude title compound was used in the next step without purification, 495 mg (67%). HPLC/MS (Method C) t_{R} 1.07 minute, M+H 693.1.

### Example 27

### rac-N-{5-(1-Hydroxy-ethyl)-4′-[2-imino-3-(1H-indol-4-ylmethyl)-2,3-dihydro-benzoimidazol-1-yl]biphenyl-2-yl}-acetamide

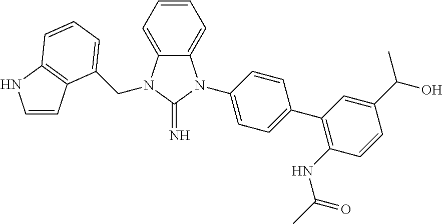 [SMILES: CC(=O)Nc1ccc(C(C)O)cc1-c1ccc(-n2c(=N)n(Cc3cccc4[nH]ccc34)c3ccccc32)cc1]

To a solution of N-{5-Acetyl-4′-[2-imino-3-(1H-indol-4-ylmethyl)-2,3-dihydro-benzoimidazol-1-yl]-biphenyl-2-yl}-acetamide (example 25.1, 50.2 mg, 0.082 mmol) in MeOH (1.0 mL) was added NaBH4 (5.6 mg, 0.147 mmol) and the resulting mixture stirred for 10 minutes at rt. The medium was then evaporated to dryness and partitioned between CH2Cl2 (5 mL) and 1M aqueous sodium hydroxide solution (5 mL). The phases were separated, the aqueous portion was extracted with CH2Cl2 (5 mL) and the combined organic phases were washed with brine (5 mL) before drying over Na2SO4 and concentration. The resulting blue solid was shown to be the title compound, 48 mg (95%) which was not purified further. HPLC/MS (Method A) t_{R} 1.01 minute, M+H 516.1. 1H NMR (dimethylsulfoxide-d6) Ppm 1.40 (d, 3H) 1.93 (s, 3H) 4.78 (m, 1H) 5.20 (d, 1H) 5.40 (s, 2H) 6.8-7.61 (m, 16H) 9.35 (s, 1H) 11.20 (s, 1H).

### Example 28

### 6-Acetylamino-4′-[2-imino-3-(1H-indol-4-ylmethyl)-2,3-dihydro-benzoimidazol-1-yl]-biphenyl-3-carboxylic acid dimethylamide

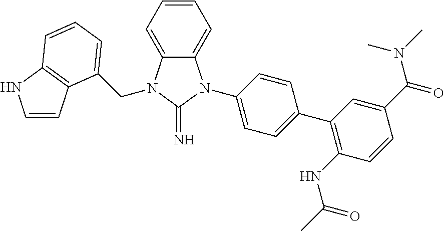 [SMILES: CC(=O)Nc1ccc(C(=O)N(C)C)cc1-c1ccc(-n2c(=N)n(Cc3cccc4[nH]ccc34)c3ccccc32)cc1]

A mixture of 4-[3-(4-Bromo-phenyl)-2-imino-2,3-dihydro-benzoimidazol-1-ylmethyl]-indole-1-carboxylic acid tert-butyl ester (intermediate O, 60 mg, 0.116 mmol), 4-Acetylamino-N,N-dimethyl-3-(4,4,5,5-tetramethyl-[1,3,2]dioxaborolan-2-yl)-benzamide (obtained from 4-Acetylamino-3-bromo-N,N-dimethyl-benzamide in a manner analogous to that used for the synthesis of intermediate U, 58 mg, 0.174 mmol) and Pd(Ph3P)4 (6.7 mg, 5.8 μmol) in DME (1.1 mL) and 2M aqueous sodium carbonate solution (0.29 mL, 0.58 mmol) in a sealed pressure vial was stirred for 1 hour at 90° C. The reaction mixture was then filtered through a pad of diatomaceous earth which was rinsed with EtOAc (3×3 mL). The resulting organic filtrate was evaporated to dryness. The residue was purified by silica gel chromatography using a gradient elution from 0% eluent B (EtOAc/MeOH/(30% aqueous NH4OH): 89/10/1) in eluent A (EtOAc+5% (7N NH3 in MeOH)) to 100% eluent B in eluent A. The product-containing fractions were pooled and concentrated. The residue was taken up in CH2Cl2 (0.7 mL) and treated with TFA (0.3 mL) for one hour at rt. The medium was then diluted with CH2Cl2 (5 mL) and washed with saturated aqueous sodium bicarbonate solution (3×5 mL) and brine (5 mL). The organic phase was dried over Na2SO4 and concentrated to give the title compound as a grey solid, 48 mg (76%). HPLC/MS (Method A) t_{R} 0.99 min, M+H 543.1. ¹H NMR (dmso-d6) Ppm 1.98 (s, 3H) 3.01 (s, 6H) 5.42 (s, 2H) 6.75-7.66 (m, 16H) 9.45 (s, 1H) 11.21 (s, 1H).

### Example 29

### 6-Acetylamino-4′-[2-imino-3-(1H-indol-4-ylmethyl)-2,3-dihydro-benzoimidazol-1-yl]-biphenyl-3-carboxylic acid amide

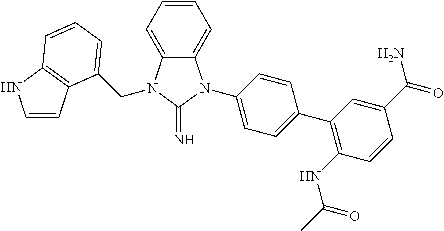 [SMILES: CC(=O)Nc1ccc(C(N)=O)cc1-c1ccc(-n2c(=N)n(Cc3cccc4[nH]ccc34)c3ccccc32)cc1]

The title compound was synthesized in a manner analogous to that used for the synthesis of example 28, using 4-Acetylamino-3-(4,4,5,5-tetramethyl-[1,3,2]dioxaborolan-2-yl)-benzamide (obtained from 4-Acetylamino-3-bromo-benzamide in a manner analogous to that used for the synthesis of intermediate U) instead of 4-Acetylamino-N,N-dimethyl-3-(4,4,5,5-tetramethyl-[1,3,2]dioxaborolan-2-yl)-benzamide. HPLC/MS (Method A) t_{R} 0.95 min, M+H 515.1. ¹H NMR (dmso-d₆) Ppm 1.98 (s, 3H) 5.54 (s, 2H) 6.7-8.05 (m, 18H) 9.45 (s, 1H) 11.25 (s, 1H).

### Example 30

### N-(6-Chloro-2-{4-[2-imino-3-(1H-indol-4-ylmethyl)-2,3-dihydro-benzoimidazol-1-yl]-phenyl}-pyridin-3-yl)-acetamide

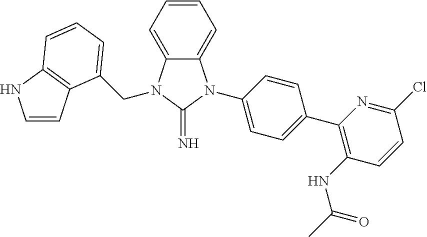 [SMILES: CC(=O)Nc1ccc(Cl)nc1-c1ccc(-n2c(=N)n(Cc3cccc4[nH]ccc34)c3ccccc32)cc1]

A mixture of intermediate V (40 mg, 0.083 mmol), N-(2-Bromo-6-chloro-pyridin-3-yl)-acetamide (obtained by trivial acetylation of commercially available 2-bromo-6-chloropyridin-3-ylamine, 22.7 mg, 0.091 mmol) and Pd(Ph3P)4 (4.8 mg, 4.1 μmol) in DME (0.41 mL) and 2M aqueous sodium carbonate solution (0.20 mL, 0.415 mmol) in a sealed pressure vial was stirred for 1 hour at 90° C. The reaction mixture was then diluted with EtOAc (2 mL) and filtered through a pad of diatomaceous earth which was rinsed with EtOAc (2×2 mL). The resulting organic filtrate was evaporated to dryness. The crude product was purified by reverse-phase preparative HPLC (Method E, gradient from 45% ((MeCN/MeOH:1/3)+0.1% TFA) in (Water+0.1% TFA) to 75% over 14 minutes). Product-containing fractions were pooled and evaporated to dryness. The residue was treated with TFA (1 mL) for 5 minutes at rt before the medium was quenched by the addition of water (1 mL) and MeOH (1 mL). The solution was then evaporated to dryness and the residue was partitioned between CH2Cl2 and saturated aqueous sodium bicarbonate solution to finally obtain the title product as a free base, 14 mg (33%). HPLC/MS (Method A) t_{R} 1.12 minute, M+H 507.2-509.2. 1H NMR (Acetone-d6) Ppm 2.04 (s, 3H) 5.40 (s, 2H) 6.72-6.96 (m, 7H) 7.06 (t, 1H) 7.30-7.34 (m, 2H) 7.55 (d, 1H) 7.70 (br. s., 2H) 7.87 (br. s., 2H) 8.08 (d, 1H) 9.83 (s, 1H) 11.20 (br. s., 1H).

### Example 31

### N-(5-Chloro-3-{4-[2-imino-3-(1H-indol-4-ylmethyl)-2,3-dihydro-benzoimidazol-1-yl]-phenyl}-pyridin-2-yl)-acetamide

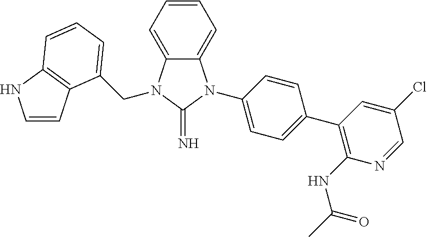 [SMILES: CC(=O)Nc1ncc(Cl)cc1-c1ccc(-n2c(=N)n(Cc3cccc4[nH]ccc34)c3ccccc32)cc1]

A mixture of intermediate V (40 mg, 0.083 mmol), N-(3-Bromo-5-chloro-pyridin-2-yl)-acetamide (obtained by trivial acetylation of commercially available 3-Bromo-5-chloropyridin-2-ylamine, 22.7 mg, 0.091 mmol) and Pd(Ph3P)4 (4.8 mg, 4.1 μmol) in DME (0.41 mL) and 2M aqueous sodium carbonate solution (0.20 mL, 0.415 mmol) in a sealed pressure vial was stirred for 1 hour at 90° C. The reaction mixture was then diluted with EtOAc (2 mL) and filtered through a pad of diatomaceous earth which was rinsed with EtOAc (2×2 mL). The resulting organic filtrate was evaporated to dryness. The crude product was purified by reverse-phase preparative HPLC (Method E, gradient from 45% ((MeCN/MeOH:1/3)+0.1% TFA) in (Water+0.1% TFA) to 75% over 14 minutes). Product-containing fractions were pooled and evaporated to dryness. The residue was treated with TFA (1 mL) for 5 minutes at rt before the medium was quenched by the addition of water (1 mL) and MeOH (1 mL). The solution was then evaporated to dryness and the residue was partitioned between CH2Cl2 and saturated aqueous sodium bicarbonate solution to finally obtain the title product as a free base, 6 mg (14%). HPLC/MS (Method A) t_{R} 1.12 minute, M+H 507.2-509.2.

### Example 32

### N-{4′-[3-(2-Chloro-benzyl)-2-imino-2,3-dihydro-benzoimidazol-1-yl]-3′-methyl-biphenyl-2-yl}-acetamide

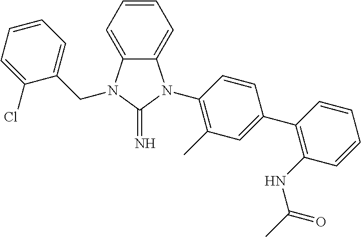 [SMILES: CC(=O)Nc1ccccc1-c1ccc(-n2c(=N)n(Cc3ccccc3Cl)c3ccccc32)c(C)c1]

To a suspension of N-[4′-(2-Amino-benzoimidazol-1-yl)-3′-methyl-biphenyl-2-yl]-acetamide (step 1, 50 mg, 0.140 mmol) in MeCN (2 mL) were added potassium iodide (23 mg, 0.14 mmol) and 2-chlorobenzylbromide (18 μL, 0.14 mmol). The resulting slurry was heated to 110° C. for 10 min under microwave irradiation. The residue was taken up in CH2Cl2 (6 mL) and washed with 0.1M aqueous sodium hydroxide solution (3×5 mL) and brine (5 mL). The crude product was purified by reverse-phase preparative HPLC (Method E, gradient from 20% (MeCN+0.1% TFA) in (Water+0.1% TFA) to 80% over 14 minutes). Product-containing fractions were pooled and evaporated to dryness to furnish the title product as its TFA salt, 18 mg (21%). HPLC/MS (method A) t_{R} 1.24 min., M+H 481.0-483.0; ¹H NMR (dmso-d6) Ppm 1.95 (s, 3H) 2.19 (s, 3H) 5.62 (s, 2H) 6.98 (d, 1H) 7.15 (d, 1H) 7.39 (m, 9H) 7.55 (d, 1H) 7.62 (m, 3H) 7.73 (d, 1H) 8.94 (s, 2H) 9.20 (s, 1H)

### Step 1: N-[4′-(2-Amino-benzoimidazol-1-yl)-3′-methyl-biphenyl-2-yl]-acetamide

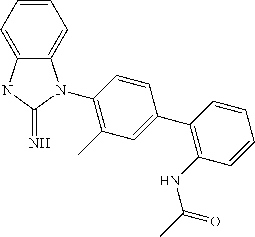 [SMILES: CC(=O)Nc1ccccc1-c1ccc(-n2c(=N)[nH]c3ccccc32)c(C)c1]

A mixture of intermediate D (200 mg, 0.662 mmol), 2-Acetamidophenylboronic acid (130 mg, 0.728 mmol) and Pd(Ph3P)₄ (38.2 mg, 33 μmol) in DME (3.3 mL) and 2M aqueous sodium carbonate solution (1.65 mL, 3.31 mmol) in a sealed pressure vial was stirred for 2 hours at 90° C. The reaction mixture was then diluted with MeOH (2 mL) and filtered through a pad of diatomaceous earth which was rinsed with EtOAc (2×10 mL). The resulting organic filtrate was evaporated to dryness. The residue was taken up in CH2Cl2 (20 mL) and washed with 1M aqueous sodium hydroxide solution (3×20 mL) and brine (20 mL). The organic phqse was dried over Na2SO4 and evaporated to dryness to furnish the title compound 236 mg (95%) which was used without further purification. HPLC/MS (Method A) t_{R} 0.96 minute, M+H 357.1.

### Example 33

### N-{4′-[3-(2-Chloro-benzyl)-2-imino-2,3-dihydro-benzoimidazol-1-yl]-2′-methyl-biphenyl-2-yl}-acetamide

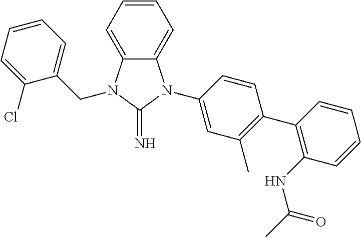 [SMILES: CC(=O)Nc1ccccc1-c1ccc(-n2c(=N)n(Cc3ccccc3Cl)c3ccccc32)cc1C]

The title compound N-{4′-[3-(2-Chloro-benzyl)-2-imino-2,3-dihydro-benzoimidazol-1-yl]-2′-methyl-biphenyl-2-yl}-acetamide was synthesized in a manner analogous to that used for the synthesis of example 32, using intermediate E instead of intermediate D in the first step. HPLC/MS (method A) t_{R} 1.24 minute, M+H 481.0-483.0. 1H NMR (Dimethylsulfoxide-d6) ppm 1.89 (s, 3H) 2.18 (s, 3H) 5.59 (s, 2H) 7.15 (m, 2H) 7.25-7.50 (m, 9H) 7.60-7.75 (m, 4H) 8.89 (bs, 2H).

### Example 34

### N-(2-{6-[3-(2-Chloro-benzyl)-2-imino-2,3-dihydro-benzoimidazol-1-yl]-pyridin-3-yl}-phenyl)-acetamide

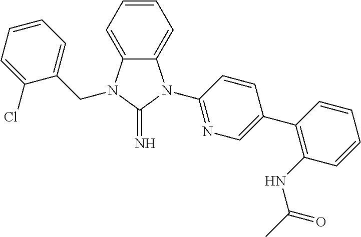 [SMILES: CC(=O)Nc1ccccc1-c1ccc(-n2c(=N)n(Cc3ccccc3Cl)c3ccccc32)nc1]

The title compound N-(2-{6-[3-(2-Chloro-benzyl)-2-imino-2,3-dihydro-benzoimidazol-1-yl]-pyridin-3-yl}-phenyl)-acetamide was synthesized in a manner analogous to that used for the synthesis of example 2, using intermediate W instead of intermediate M. HPLC/MS (method A) t_{R} 1.13 min, M+H 468.

### Example 35

### N-(2-{6-[3-(2-Chloro-benzyl)-2-imino-2,3-dihydro-benzoimidazol-1-yl]-pyridin-3-yl}-phenyl)-N-methyl-acetamide

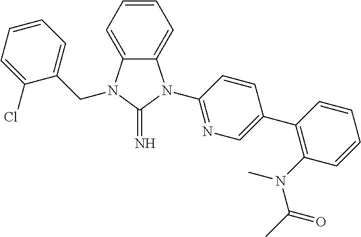 [SMILES: CC(=O)N(C)c1ccccc1-c1ccc(-n2c(=N)n(Cc3ccccc3Cl)c3ccccc32)nc1]

The title compound N-(2-{6-[3-(2-Chloro-benzyl)-2-imino-2,3-dihydro-benzoimidazol-1-yl]-pyridin-3-yl}-phenyl)-N-methyl-acetamide was synthesized in a manner analogous to that used for the synthesis of example 2, using intermediate W instead of intermediate M and N-Methyl-N-[2-(4,4,5,5-tetramethyl-[1,3,2]dioxaborolan-2-yl)-phenyl]-acetamide (obtained by methylation of N-[2-(4,4,5,5-Tetramethyl-[1,3,2]dioxaborolan-2-yl)-phenyl]-acetamide by methyl iodide in the presence of potassium carbonate in DMF at rt) instead of 2-acetamidophenylboronic acid. HPLC/MS (method A) t_{R} 1.13 min, M+H 482.

### Example 36

### N-{5-Chloro-4′-[3-(2-chloro-benzyl)-2-imino-7-methyl-2,3-dihydro-benzoimidazol-1-yl]-biphenyl-2-yl}-acetamide

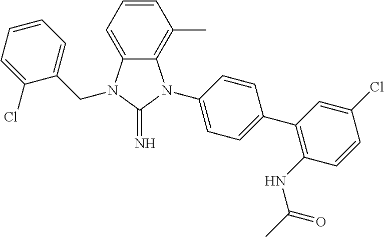 [SMILES: CC(=O)Nc1ccc(Cl)cc1-c1ccc(-n2c(=N)n(Cc3ccccc3Cl)c3cccc(C)c32)cc1]

The title compound was synthesized in a manner analogous to that used for the synthesis of example 1, using intermediate Z instead of intermediate M. HPLC/MS (Method A) t_{R} 1.40 min, M+H 515-517.

### Example 37

### N-{4′-[3-(2-Chloro-benzyl)-2-imino-7-methyl-2,3-dihydro-benzoimidazol-1-yl]-5-methyl-biphenyl-2-yl}-acetamide

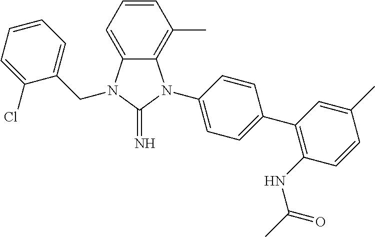 [SMILES: CC(=O)Nc1ccc(C)cc1-c1ccc(-n2c(=N)n(Cc3ccccc3Cl)c3cccc(C)c32)cc1]

The title compound was synthesized in a manner analogous to that used for the synthesis of example 1, using intermediate Z instead of intermediate M and intermediate X instead of 2-acetamido-5-chlorophenyl boronic acid pinacol ester. HPLC/MS (Method A) t_{R} 1.330 minute, M+H 495-497.

### Example 38

### N-[4′-(3-Benzyl-2-imino-7-methyl-2,3-dihydro-benzoimidazol-1-yl)-5-methyl-biphenyl-2-yl]-acetamide

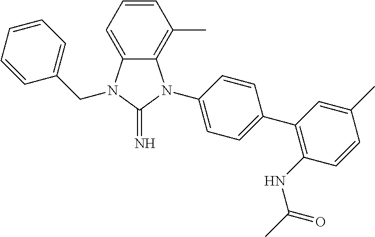 [SMILES: CC(=O)Nc1ccc(C)cc1-c1ccc(-n2c(=N)n(Cc3ccccc3)c3cccc(C)c32)cc1]

A suspension of intermediate Y (50.0 mg, 0.135 mmol), benzyl bromide (16.0 μL, 0.135 mmol), and KI (22.4 mg, 0.135 mmol) in acetonitrile (675 μl) was stirred at 110° C. for 10 minutes under microwave irradiation. The reaction mixture was then concentrated to dryness under vacuum. The crude was dissolved in EtOAc (5 mL) and washed with saturated sodium bicarbonate solution (2×5 mL). The organic phase was dried over Na2SO4, filtered and concentrated to a brown oil. The crude product was purified by reverse-phase preparative HPLC (Method E, gradient from 20% (MeCN+0.1% TFA) in (Water+0.1% TFA) to 80% over 14 minutes). Product-containing fractions were pooled and evaporated to dryness to furnish the title product as its TFA salt, 25 mg (32%). HPLC/MS (Method A) t_{R} 1.31 minute, M+H 461.0.

### Example 40

### N-{5-Chloro-4′-[2-imino-3-(1H-indol-4-ylmethyl)-7-methyl-2,3-dihydro-benzoimidazol-1-yl]-biphenyl-2-yl}-acetamide

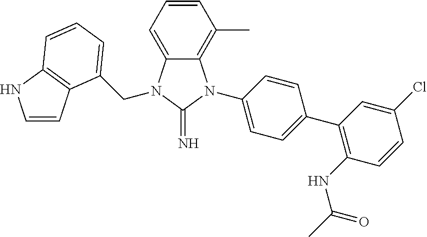 [SMILES: CC(=O)Nc1ccc(Cl)cc1-c1ccc(-n2c(=N)n(Cc3cccc4[nH]ccc34)c3cccc(C)c32)cc1]

A mixture composed of intermediate AA (50 mg, 0.094 mmol), 2-Acetamido-5-chlorophenylboronic acid, pinacol ester (27.8 mg, 0.094 mmol), Pd(PPh3)2Cl2 (3.30 mg, 4.70 μmol) and Na2CO3 2M (235 μl, 0.470 mmol) in DME (470 μl) was heated to 150° C. for 16 min under microwave irradiation. The medium was then diluted with EtOAc (5 mL) and saturated sodium bicarbonate solution (5 mL). The phases were separated, the aqueous phase was washed with EtOAc (2 ml), the organics dried over Na2SO4 and concentrated. The residue was treated for 5 minutes with TFA (4 mL) before quenching with water (10 mL). The medium was diluted with CH2Cl2 (25 mL) and water (25 mL) then basified using solid NaHCO3. The phases were separated, the aqueous portion was washed with CH2Cl2 (3×25 mL), the combined organics were dried over Na2SO4, and evaporated to dryness. The crude product was purified by reverse-phase preparative HPLC (Method E, gradient from 20% (MeCN+0.1% TFA) in (Water+0.1% TFA) to 80% over 14 minutes). Product-containing fractions were pooled and evaporated to dryness. The residue was partitioned between AcOEt (5 mL) and saturated sodium bicarbonate solution (5 mL) to effect the removal of the TFA salt and furnish, after drying of the organic portion, the title compound as a white solid, 16 mg (33%). HPLC/MS (Method A) t_{R} 1.24 minute, M+H 520.0-522.0. 1H NMR (Acetone-d6) Ppm 1.85 (s, 3H) 2.01 (s, 3H) 5.46 (s, 2H) 6.55-6.68 (m, 1H) 6.70-6.83 (m, 2H) 6.91 (d, J=2.45 Hz, 1H) 7.01-7.17 (m, 2H) 7.28-7.50 (m, 4H) 7.54-7.65 (m, 2H) 7.66-7.79 (m, 2H) 7.87-8.09 (m, 1H) 8.60 (br. s., 1H) 10.36 (br. s., 1H).

The following examples 40.1 to 40.2 were synthesized in a manner analogous to that used for the synthesis of example 40, from intermediate AA and various aryl boronic acids or boronate esters.

| Ex. | structure | name | HPLC* | MS** | 1H NMR*** |
|---|---|---|---|---|---|
| 40.1 | 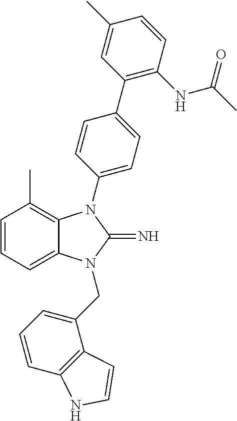 [SMILES: CC(=O)Nc1ccc(C)cc1-c1ccc(-n2c(=N)n(Cc3cccc4[nH]ccc34)c3cccc(C)c32)cc1] | N-{4′-[2-Imino-3-(1H- indol-4-ylmethyl)- 7-methyl-2,3- dihydro-benzoimi- dazol-1-yl]-5-methyl- biphenyl-2-yl}-aceta- mide | 1.18 (A) | 500.0 | |
| 40.2 | 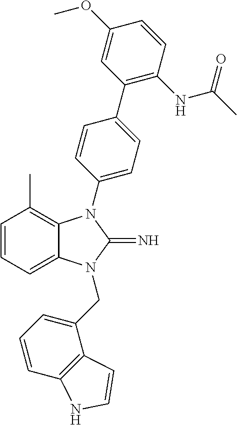 [SMILES: COc1ccc(NC(C)=O)c(-c2ccc(-n3c(=N)n(Cc4cccc5[nH]ccc45)c4cccc(C)c43)cc2)c1] | N-{4′-[2-Imino-3-(1H- indol-4-ylmethyl)- 7-methyl-2,3- dihydro-benzoimi- dazol-1-yl]-5-methoxy- biphenyl-2-yl}- acetamide | 1.14 (A) | 516.0 | |

*t_{R} [min] (method);
**M + H (or specified);
***d⁶-dmso

### Example 41

### N-{5-Chloro-4′-[3-(2-chloro-benzyl)-5,6-difluoro-2-imino-2,3-dihydro-benzoimidazol-1-yl]-biphenyl-2-yl}-acetamide

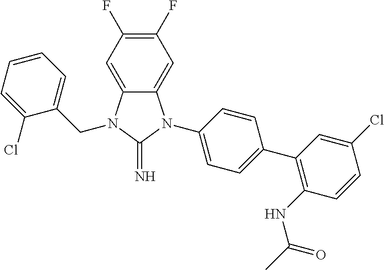 [SMILES: CC(=O)Nc1ccc(Cl)cc1-c1ccc(-n2c(=N)n(Cc3ccccc3Cl)c3cc(F)c(F)cc32)cc1]

The title compound was synthesized in a manner analogous to that used for the synthesis of example 32, using intermediate AB instead of N-[4′-(2-Amino-benzoimidazol-1-yl)-3′-methyl-biphenyl-2-yl]-acetamide. HPLC/MS (Method D) t_{R} 2.38 minute, M+H 537.0-539.0.

### Example 42

### N-{5-Chloro-4′-[5,6-difluoro-2-imino-3-(1H-indol-4-ylmethyl)-2,3-dihydro-benzoimidazol-1-yl]biphenyl-2-yl}-acetamide

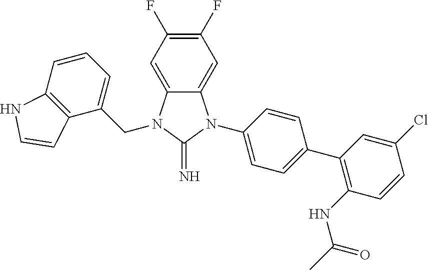 [SMILES: CC(=O)Nc1ccc(Cl)cc1-c1ccc(-n2c(=N)n(Cc3cccc4[nH]ccc34)c3cc(F)c(F)cc32)cc1]

The title compound was synthesized in a manner analogous to that used for the synthesis of example 38, using intermediate AB instead of intermediate Y and intermediate N instead of benzyl bromide, removing the Boc protecting group as described in the synthesis of example 40 (4 minute treatment with TFA before water quenching). HPLC/MS (Method A) t_{R} 1.23 minute, M+H 542.0-544.0.

### Example 43

### N-(4-Chloro-2-{6-[3-(2-chloro-benzyl)-2-imino-2,3-dihydro-benzoimidazol-1-yl]-pyridazin-3-yl}-phenyl)-acetamide

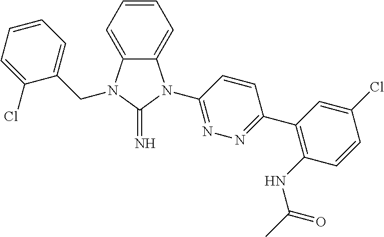 [SMILES: CC(=O)Nc1ccc(Cl)cc1-c1ccc(-n2c(=N)n(Cc3ccccc3Cl)c3ccccc32)nn1]

The title compound was synthesized in a manner analogous to that used for the synthesis of example 1, using intermediate AC instead of intermediate M. HPLC/MS (Method A) t_{R} 1.11 minute, M+H 502.9-504.9. 1H NMR (TFA salt) (Dimethylsulfoxide-d6) Ppm 2.03 (s, 3H) 5.63 (s, 2H) 7.20 (dd, 1H) 7.36 (td, 1H) 7.39-7.52 (m, 5H) 7.62-7.71 (m, 2H) 7.85 (d, 1H) 7.96 (d, 1H) 8.40 (d, 1H) 8.51 (d, 1H) 9.42 (br. s., 2H) 10.35 (br. s., 1H).

The following examples 43.1 to 43.3 were synthesized in a manner analogous to that used for the synthesis of example 43, from intermediate AC and various aryl boronic acids or boronate esters.

| | | | | MS | |
|---|---|---|---|---|---|
| Ex. | structure | name | HPLC* | M + H** | 1H NMR*** |
| 43.1 | 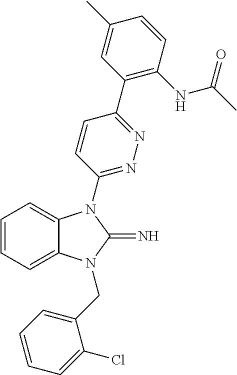 [SMILES: CC(=O)Nc1ccc(C)cc1-c1ccc(-n2c(=N)n(Cc3ccccc3Cl)c3ccccc32)nn1] | N-(2-{6-[3-(2- Chloro-benzyl)- 2-imino- 2,3-dihydro- benzoimidazol- 1-yl]-pyri- dazin-3-yl}-4- methyl-phenyl)- acetamide | 1.07 (A) | 483.0- 485.0 | Ppm 2.00 (s, 3 H) 2.42 (s, 3 H) 5.63 (s, 2 H) 7.20 (dd, 1 H) 7.29-7.52 (m, 8 H) 7.60-7.67 (m, 2 H) 7.78 (d, 1 H) 8.37 (d, 1 H) 8.42 (d, 1 H) 9.41 (br. s., 2 H) 10.23 (br. s., 1 H), TFA salt |
| 43.2 | 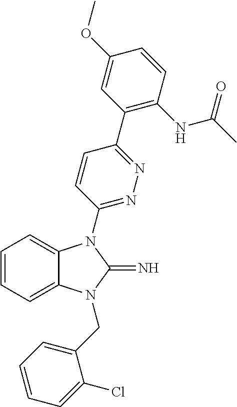 [SMILES: COc1ccc(NC(C)=O)c(-c2ccc(-n3c(=N)n(Cc4ccccc4Cl)c4ccccc43)nn2)c1] | N-(2-{6-[3-(2- Chloro-benzyl)- 2-imino- 2,3-dihydro- benzoimidazol- 1-yl]-pyri- dazin-3-yl}-4- methoxy- phenyl)-ace- tamide | 1.01 (A) | 499.0- 501.0 | Ppm 1.98 (s, 3 H) 3.86 (s, 3 H) 5.63 (s, 2 H) 7.07-7.25 (m, 2 H) 7.28-7.56 (m, 8 H) 7.59-7.74 (m, 2 H) 8.31-8.44 (m, 2 H) 9.42 (br. s, 2 H) 9.98 (s, 1 H), TFA salt |
| 43.3 | 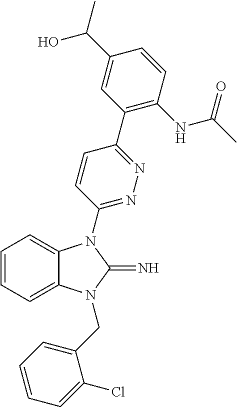 [SMILES: CC(=O)Nc1ccc(C(C)O)cc1-c1ccc(-n2c(=N)n(Cc3ccccc3Cl)c3ccccc32)nn1] | Rac-N-[2-{6-[3- (2-Chloro- benzyl)-2-imino- 2,3-dihydro- benzoimidazol- 1-yl]-pyri- dazin-3-yl}-4- (1-hydroxy- ethyl)- phenyl]- acetamide | 0.98 (A) | 513.0- 515.0 | Ppm 1.40 (d, 3 H) 2.01 (s, 3 H) 4.84 (q, 1 H) 5.63 (s, 2 H) 7.20 (dd, 1 H) 7.32-7.50 (m, 6 H) 7.53 (dd, 1 H) 7.64 (dd, 1 H) 7.72- 7.84 (m, 2 H) 8.24- 8.45 (m, 2 H) 9.41 (br. s., 2 H) 10.20 (s, 1 H) |

*t_{R} [min] (method);
**M + H (or specified);
***d⁶-dmso

### Example 44

### N-(2-{6-[2-Imino-3-(1H-indol-4-ylmethyl)-2,3-dihydro-benzoimidazol-1-yl]-pyridazin-3-yl}-phenyl)-acetamide

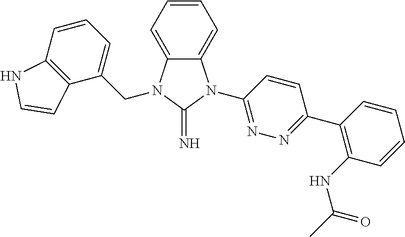 [SMILES: CC(=O)Nc1ccccc1-c1ccc(-n2c(=N)n(Cc3cccc4[nH]ccc34)c3ccccc32)nn1]

The title compound was synthesized in a manner analogous to that used for the synthesis of example 24, using intermediate AD instead of intermediate 0. HPLC/MS (Method D) t_{R} 1.53 minute, M+H 473.9. 1H NMR (TFA salt) (Acetone-d₆) 1H NMR (400 MHz, Acetone) Ppm 2.11 (s, 3H) 6.03 (s, 2H) 6.64-6.80 (m, 1H) 6.97 (d, 1H) 7.12 (t, 1H) 7.35 (td, 1H) 7.41-7.54 (m, 4H) 7.54-7.63 (m, 2H) 7.64-7.73 (m, 1H) 7.87 (dd, 1H) 8.39 (d, 1H) 8.47-8.62 (m, 2H) 9.61 (br. s., 1H) 10.65 (br. s., 1H) 10.89 (br. s., 1H)

The following examples 44.1 to 44.2 were synthesized in a manner analogous to that used for the synthesis of example 44, from intermediate AD and various aryl boronic acids or boronate esters.

| Ex. | structure | name | HPLC* | MS** | 1H NMR*** |
|---|---|---|---|---|---|
| 44.1 | 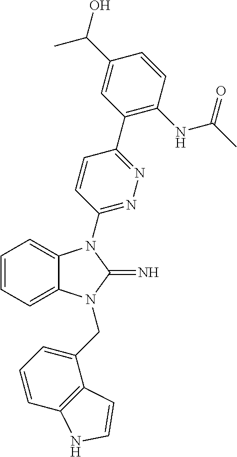 [SMILES: CC(=O)Nc1ccc(C(C)O)cc1-c1ccc(-n2c(=N)n(Cc3cccc4[nH]ccc34)c3ccccc32)nn1] | N-(4-(1-Hydroxy- ethyl)-2-{6-[2-imino- 3-(1H-indol-4- ylmethyl)-2,3-dihydro- benzoimidazol- 1-yl]-pyridazin-3- yl}-phenyl)- acetamide | 1.18 (D) | 517.9 | 3.17 (d, 3H) 2.12 (s, 3H) 4.96 (q, 1H) 6.03 (s, 2H) 6.72-8.48 (m, 15H) 10.70 (2s, 2H) |
| 44.2 | 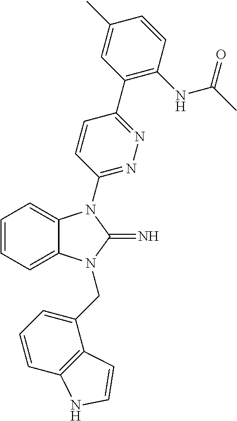 [SMILES: CC(=O)Nc1ccc(C)cc1-c1ccc(-n2c(=N)n(Cc3cccc4[nH]ccc34)c3ccccc32)nn1] | N-(2-{6-[2-Imino-3- (1H-indol-4-ylmeth- yl)-2,3-dihydro- benzoimidazol-1-yl]- pyridazin-3-yl}-4- methyl-phenyl)- acetamide | 1.77 (D) | 487.8 | |

*t_{R} [min] (method);
**M + H (or specified);
***d⁶-acetone

### Example 45

### N-(4-Acetyl-2-{6-[3-(2-chloro-benzyl)-2-imino-7-methyl-2,3-dihydro-benzoimidazol-1-yl]-pyridazin-3-yl}-phenyl)-acetamide

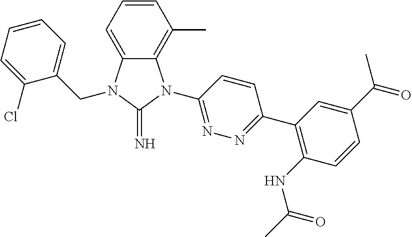 [SMILES: CC(=O)Nc1ccc(C(C)=O)cc1-c1ccc(-n2c(=N)n(Cc3ccccc3Cl)c3cccc(C)c32)nn1]

The title compound was synthesized in a manner analogous to that used for the synthesis of example 15, using intermediate AE instead of intermediate M. HPLC/MS (Method D) t_{R} 2.03 minute, M+H 524.8-526.8. 1H NMR (TFA salt) (dmso-d₆) Ppm 1.91 (s, 3H) 2.00 (s, 3H) 2.67 (s, 3H) 5.61 (s, 2H) 7.17 (m, 2H) 7.26-7.46 (m, 4H) 7.64 (d, 1H) 8.01 (d, 1H) 8.20 (d, 1H) 8.33 (s, 1H) 8.49 (d, 1H) 8.55 (d, 1H) 9.06 (bs, 2H) 10.72 (s, 1H).

The following examples 45.1 to 45.2 were synthesized in a manner analogous to that used for the synthesis of example 45, from intermediate AE and various aryl boronic acids or boronate esters (obtained from the corresponding aryl bromides as depicted in the synthesis of example 15).

| Ex. | structure | name | HPLC* | MS** | 1H NMR*** |
|---|---|---|---|---|---|
| 45.1 | 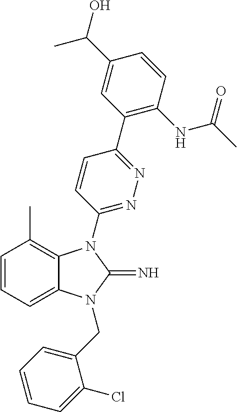 [SMILES: CC(=O)Nc1ccc(C(C)O)cc1-c1ccc(-n2c(=N)n(Cc3ccccc3Cl)c3cccc(C)c32)nn1] | Rac-N-[2-{6-[3-(2- Chloro-benzyl)-2- imino- 7-methyl-2,3- dihydro- benzoimidazol- 1-yl]-pyridazin-3- yl}-4-(1-hydroxy- ethyl)-phenyl]- acetamide | 1.02 (A) | 527.0- 529.0 | 1.41 (d, 3H) 1.87 (s, 3H) 1.91 (s, 3H) 4.85 (m, 1H) 5.60 (s, 2H) 7.13- 7.65 (m, 10H) 7.76 (s, 1H) 8.34 (d, 1H) 8.42 (d, 1H) 9.07 (bs, 2H) 10.24 (s, 1H), TFA salt |
| 45.2 | 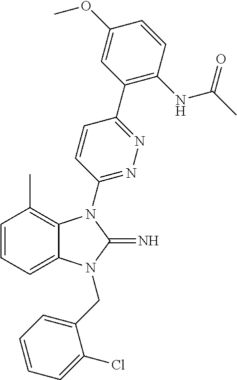 [SMILES: COc1ccc(NC(C)=O)c(-c2ccc(-n3c(=N)n(Cc4ccccc4Cl)c4cccc(C)c43)nn2)c1] | N-(2-{6-[3-(2- Chloro-benzyl)-2- imino- 7-methyl-2,3- dihydro- benzoimidazol- 1-yl]-pyridazin-3- yl}-4-methoxy-phe- nyl)-acetamide | 1.13 (A) | 513.0- 515.0 | Recorded in Ace- ton-d6 Ppm 1.96 (s, 3H) 2.02 (s, 3H) 3.89 (s, 3H) 5.82 (s, 2H) 7.13-7.46 (m, 8H) 7.59 (d, 1H) 7.99 (m, 1H) 8.42 (dd, 2H) 9.68 (bs, 2H) 10.35 (s, 1H), TFA salt |

*t_{R} [min] (method);
**M + H (or specified);
***d⁶-dmso or otherwise specified solvent

### Example 46

### N-(4-Chloro-2-{6-[2-imino-3-(1H-indol-4-ylmethyl)-7-methyl-2,3-dihydro-benzoimidazol-1-yl]-pyridazin-3-yl}-phenyl)-acetamide

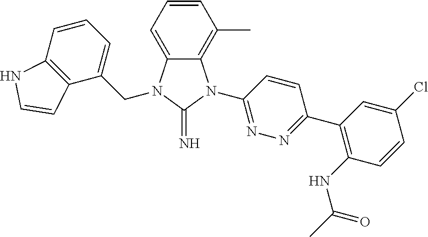 [SMILES: CC(=O)Nc1ccc(Cl)cc1-c1ccc(-n2c(=N)n(Cc3cccc4[nH]ccc34)c3cccc(C)c32)nn1]

The title compound was synthesized in a manner analogous to that used for the synthesis of example 25, using intermediate AF instead of intermediate 0. HPLC/MS (Method A) t_{R} 1.12 min., M+H 522.0-524.0; ¹H NMR (acetone-d₆) ppm 1.96 (s, 3H) 2.10 (s, 3H) 5.45 (s, 2H) 6.77 (d, 2H) 6.85 (d, 1H) 6.93 (t, 1H) 7.03 (d, 1H) 7.09 (m, 1H) 7.39 (m, 2H) 7.55 (dd, 1H) 7.94 (d, 1H) 8.23 (s, 1H) 8.45 (d, 1H) 8.51 (d, 1H) 10.46 (s, 1H) 11.36 (s, 1H).

The following examples 46.1 to 46.5 were synthesized in a manner analogous to that used for the synthesis of example 46, from intermediate AF and various aryl boronic acids or boronate esters (purchased or obtained from the corresponding aryl bromides as depicted in the synthesis of example 15).

| Ex. | structure | name | HPLC | MS** | 1H NMR*** |
|---|---|---|---|---|---|
| 46.1 | 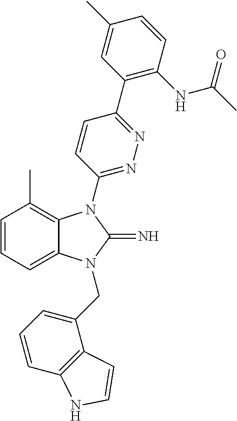 [SMILES: CC(=O)Nc1ccc(C)cc1-c1ccc(-n2c(=N)n(Cc3cccc4[nH]ccc34)c3cccc(C)c32)nn1] | N-(2-{6-[2-Imino-3- (1H-indol-4-ylmeth- yl)-7-methyl-2,3- dihydro-benzoimi dazol-1-yl]- pyridazin-3-yl}-4- methyl- phenyl)-acetamide | 1.08 (A) | 502.0 | Ppm 1.84 (s, 3H) 1.91 (s, 3H) 2.43 (s, 3H) 5.82 (s, 2H) 6.64 (s, 1H) 6.84 (d, 1H) 7.10 (m, 2H) 7.23 (m, 2H) 7.41 (m, 2H) 7.49 (s, 1H) 7.62 (m, 2H) 8.41 (dd, 2H) 9.11 (bs, 2H) 10.24 (s, 1H) 11.38 (s, 1H) |
| 46.2 | 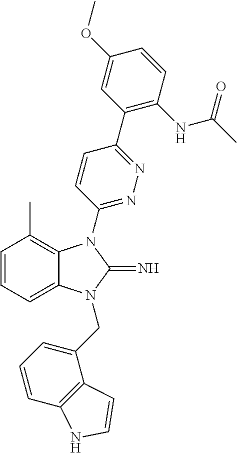 [SMILES: COc1ccc(NC(C)=O)c(-c2ccc(-n3c(=N)n(Cc4cccc5[nH]ccc45)c4cccc(C)c43)nn2)c1] | N-(2-{6-[2-Imino-3- (1H-indol-4-ylmeth- yl)-7-methyl-2,3- dihydro-benzo- imidazol-1-yl]- pyridazin-3-yl}-4- methoxy- phenyl)- acetamide | 1.03 (A) | 518.1 | Ppm 1.83 (s, 3H) 1.87 (s, 3H) 3.87 (s, 3H) 5.82 (s, 2H) 6.64 (s, 1H) 6.84 (d, 1H) 7.08- 7.31 (m, 6H) 7.41 (d, 1H) 7.53 (m, 2H) 8.36 (d, 1H) 8.45 (d, 1H) 9.12 (bs, 2H) 10.01 (s, 1H) 11.38 (s, 1H), TFA salt |
| 46.3 | 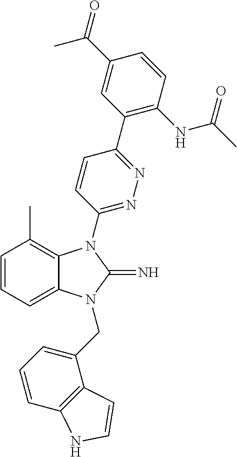 [SMILES: CC(=O)Nc1ccc(C(C)=O)cc1-c1ccc(-n2c(=N)n(Cc3cccc4[nH]ccc34)c3cccc(C)c32)nn1] | N-(4-Acetyl-2-{6-[2- imino-3-(1H-indol- 4-ylmethyl)-7- methyl-2,3-dihydro- benzoimidazol-1- yl]-pyridazin-3-yl}- phenyl)- acetamide | 1.78 (D) | 529.8 | Ppm 1.85 (s, 3H) 2.04 (s, 3H) 2.66 (s, 3H) 5.39 (s, 2H) 6.71-8.36 (m, 14H) 11.20 (s, 1H) |
| 46.4 | 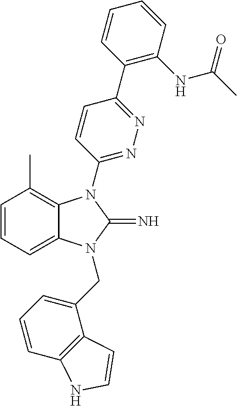 [SMILES: CC(=O)Nc1ccccc1-c1ccc(-n2c(=N)n(Cc3cccc4[nH]ccc34)c3cccc(C)c32)nn1] | N-(2-{6-[2-Imino-3- (1H-indol-4-ylmeth- yl)-7-methyl-2,3- dihydro-benzoimida- zol-1-yl]- pyridazin-3-yl}- phenyl)- acetamide | 0.99 (A) | 488.1 | |
| 46.5 | 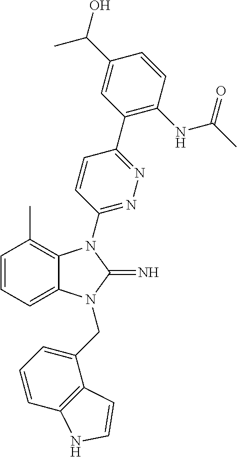 [SMILES: CC(=O)Nc1ccc(C(C)O)cc1-c1ccc(-n2c(=N)n(Cc3cccc4[nH]ccc34)c3cccc(C)c32)nn1] | N-(4-(1-Hydroxy- ethyl)-2-{6-[2-imino- 3-(1H-indol-4- ylmethyl)-7-methyl- 2,3-dihydro- benzoimidazol-1- yl]-pyri- dazin-3-yl}- phenyl)-acetamide | 1.54 (D) | 531.9 | Recorded in Ace- tone-d6 Ppm 1.50 (d, 3H) 1.98 (s, 3H) 4.95 (m, 1H) 6.00 (s, 2H) 6.72 (s, 1H) 6.95 (d, 1H) 7.11- 7.57 (m, 7H) 7.88 (s, 1H) 8.18 (m, 1H) 8.48 (q, 2H) 9.53 (s, 1H) 10.69 (bd, 1H). |

*t_{R} [min] (method);
**M + H (or specified);
***d⁶-dmso or otherwise specified solvent

### Example 47

### N-(4-Chloro-2-{5-[3-(2-chloro-benzyl)-2-imino-2,3-dihydro-benzoimidazol-1-yl]-pyridin-2-yl}-phenyl)-acetamide

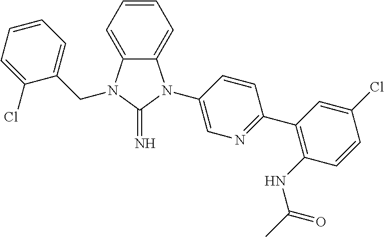 [SMILES: CC(=O)Nc1ccc(Cl)cc1-c1ccc(-n2c(=N)n(Cc3ccccc3Cl)c3ccccc32)cn1]

A mixture composed of 1-(6-Bromo-pyridin-3-yl)-3-(2-chloro-benzyl)-1,3-dihydro-benzoimidazol-2-ylideneamine (step 1, 60 mg, 0.162 mmol), 2-Acetamido-5-chlorophenylboronic acid pinacol ester (48.0 mg, 0.162 mmol), Pd(PPh₃)₂Cl₂ (5.70 mg, 8.12 μmol) and aqueous Na₂CO₃ 2M (406 μl, 0.812 mmol) in DME (812 μl) was heated to 150° C. for 16 min under microwave irradiation. The medium was diluted with EtOAc (5 ml) and aqueous saturated sodium bicarbonate solution (5 ml). The phases were separated, the aqueous phase washed with EtOAc (2 ml), the organics dried over Na2SO4 and concentrated. The crude product was purified by chromatography on silica gel, using a 25% to 100% gradient of eluent B (EtOAc/MeOH/concentrated NH₄OH:90/9/1) in eluent A (heptane), leading to the title compound as a white solid, 82 mg (77%). HPLC/MS (Method D) t_{R} 2.21 min, M+H 502.0-504.0. ¹H NMR (Aceton-d₆) Ppm 2.14 (s, 3H) 5.28 (s, 2H) 5.73 (br. s., 1H) 6.83-7.09 (m, 4H) 7.14-7.38 (m, 3H) 7.39-7.52 (m, 2H) 7.91 (d, 1H) 8.16-8.27 (m, 1H) 8.28-8.42 (m, 1H) 8.60 (d, 1H) 9.07 (d, 1H) 11.98 (br. s., 1H).

### Step 1: 1-(6-Bromo-pyridin-3-yl)-3-(2-chloro-benzyl)-1,3-dihydro-benzoimidazol-2-ylideneamine

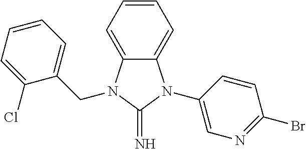 [SMILES: N=c1n(Cc2ccccc2Cl)c2ccccc2n1-c1ccc(Br)nc1]

To a suspension of intermediate B (3.02 g, 12.3 mmol) in MeCN (24.6 mL) were added potassium iodide (2.07 g, 12.3 mmol) and 2-chlorobenzylbromide (1.60 mL, 12.3 mmol). The resulting slurry was heated to 110° C. for 10 min under microwave irradiation. The medium was evaporated to dryness. The crude was taken up in water (100 mL) and CH2Cl2 (200 mL). The mixture was shaken and the phases were separated. The aqueous phase was extracted with CH2Cl2 (2×100 mL) and the organics were dried over Na2SO4 and concentrated to a crude solid. The crude product was purified by chromatography on silica gel, using a 10% to 100% gradient of eluent B (EtOAc+1% [7N NH₃ in MeOH]) in eluent A (heptane), leading to the title compound as a yellow solid, 2.60 g (57%). HPLC/MS (Method A) t_{R} 1.05 minute, M+H 368.9-370.9. ¹H NMR (DMSO-d₆) Ppm 5.20 (s, 2H) 5.84 (br. s., 1H) 6.63-7.18 (m, 5H) 7.21-7.40 (m, 2H) 7.54 (dd, 1H) 7.75 (d, 1H) 8.16 (d, 1H) 8.70 (br. s., 1H)

The following examples 47.1 to 47.9 were synthesized in a manner analogous to that used for the synthesis of example 47, from intermediate B and various aryl boronic acids or boronate esters (purchased or obtained from the corresponding aryl bromides as depicted in the synthesis of example 15).

| Ex. | structure | name | HPLC* | MS** | 1H NMR*** |
|---|---|---|---|---|---|
| 47.1 | 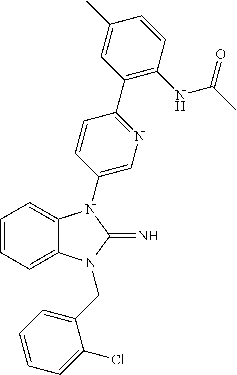 [SMILES: CC(=O)Nc1ccc(C)cc1-c1ccc(-n2c(=N)n(Cc3ccccc3Cl)c3ccccc32)cn1] | N-(2-{5-[3-(2- Chloro-benzyl)-2- imino-2,3-dihydro- benzoimidazol-1- yl]-pyridin-2-yl}-4- methyl-phenyl)- acetamide | 1.12 (A) | 482.0- 484.1 | (Acetone-d₆) Ppm 2.10 (s, 3 H) 2.41 (s, 3 H) 5.88 (s, 2 H) 7.19-7.45 (m, 7 H) 7.46-7.52 (m, 1 H) 7.54- 7.61 (m, 1 H) 7.69 (d,, 1 H) 8.22 (d, 1 H) 8.33-8.39 (m, 1 H) 8.42 (dd, 1 H) 9.17 (d, 1 H) 9.88 (br. s., 1 H) 11.37 (br. s., 1 H) |
| 47.2 | 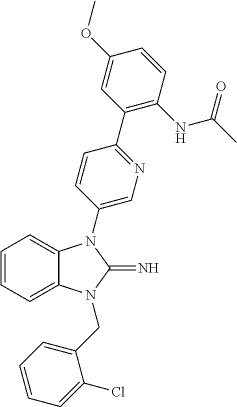 [SMILES: COc1ccc(NC(C)=O)c(-c2ccc(-n3c(=N)n(Cc4ccccc4Cl)c4ccccc43)cn2)c1] | N-(2-{5-[3-(2- Chloro-benzyl)-2- imino- 2,3-dihydro- benzoimidazol-1- yl]-pyri- din-2-yl}-4- methoxy-phenyl)- acetamide | 1.10 (A) | 498.0- 500.0 | |
| 47.3 | 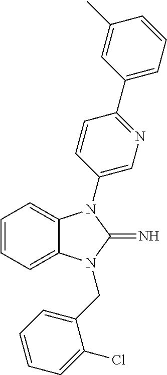 [SMILES: Cc1cccc(-c2ccc(-n3c(=N)n(Cc4ccccc4Cl)c4ccccc43)cn2)c1] | 1-(2-Chloro- benzyl)-3-(6-m- tolyl-pyri- din-3-yl)-1,3- dihydro- benzoimidazol- 2-ylideneamine | 1.30 (A) | 425.0- 427.0 | |
| 47.4 | 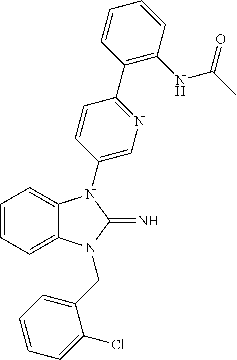 [SMILES: CC(=O)Nc1ccccc1-c1ccc(-n2c(=N)n(Cc3ccccc3Cl)c3ccccc32)cn1] | N-(2-{5-[3-(2- Chloro-benzyl)-2- imino- 2,3-dihydro- benzoimidazol-1- yl]-pyri- din-2-yl}-phenyl)- acetamide | 1.11 (A) | 468.0- 470.0 | 2.41 (s, 3 H) 5.58 (s, 2 H) 7.13- 7.25 (m, 2 H) 7.27- 7.47 (m, 7 H) 7.63 (dd, 1 H) 8.15 (d, 2 H) 8.23- 8.50 (m, 2 H) 9.01 (m, 3 H), TFA salt |
| 47.5 | 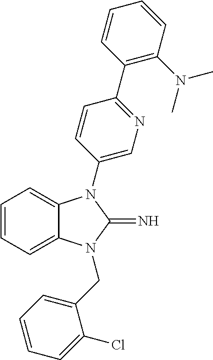 [SMILES: CN(C)c1ccccc1-c1ccc(-n2c(=N)n(Cc3ccccc3Cl)c3ccccc32)cn1] | (2-{5-[3-(2-Chloro- benzyl)-2-imino- 2,3-dihydro- benzoimidazol-1- yl]-pyri- din-2-yl}-phenyl)- dimethyl-amine | 0.85 (A) | [(M + 2H)/ 2]⁺ = 227.5 | 2.69 (s, 6 H) 5.60 (s, 2 H) 7.08- 7.27 (m, 4 H) 7.30- 7.39 (m, 3 H) 7.40-7.50 (m, 3 H) 7.59-7.76 (m, 2 H) 8.16-8.33 (m, 2 H) 9.04 (d, 1 H) 9.10 (br. s, 2 H), TFA salt |
| 47.6 | 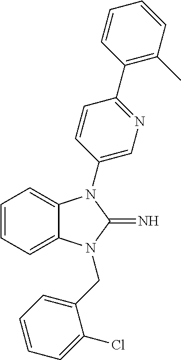 [SMILES: Cc1ccccc1-c1ccc(-n2c(=N)n(Cc3ccccc3Cl)c3ccccc32)cn1] | 1-(2-Chloro- benzyl)-3-(6-o-tolyl- pyri- din-3-yl)-1,3- dihydro- benzoimidazol- 2-ylideneamine | 1.24 (A) | 425.0- 427.0 | 2.47 (s, 3 H) 5.59 (s, 2 H) 7.14- 7.27 (m, 2 H) 7.27- 7.49 (m, 8 H) 7.55 (d, 1 H) 7.63 (dd, 1 H) 7.93 (d, 1 H) 8.32 (dd, 1 H) 8.73-9.30 (m, 3 H), TFA salt |
| 47.7 | 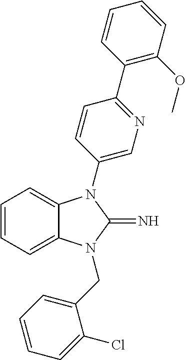 [SMILES: COc1ccccc1-c1ccc(-n2c(=N)n(Cc3ccccc3Cl)c3ccccc32)cn1] | 1-(2-Chloro- benzyl)-3-[6-(2- methoxy- phenyl)-pyridin-3- yl]-1,3-dihydro- benzoimidazol-2- ylideneamine | 1.23 (A) | 441.0- 443.0 | |
| 47.8 | 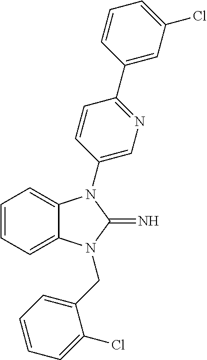 [SMILES: N=c1n(Cc2ccccc2Cl)c2ccccc2n1-c1ccc(-c2cccc(Cl)c2)nc1] | 1-(2-Chloro- benzyl)-3-[6-(3- chloro- phenyl)-pyridin-3- yl]-1,3-dihydro-benzo- imidazol-2- ylideneamine | 1.30 (A) | 444.9- 447.0 | 5.59 (s, 2 H) 7.10- 7.26 (m, 2 H) 7.30-7.39 (m, 3H) 7.40-7.51 (m, 2H) 7.57- 7.68 (m, 3H) 8.18- 8.26 (m, 1H) 8.28-8.33 (m, 1H) 8.38 (dd, 1H) 8.44-8.52 (m, 1H) 9.00-9.13 (m, 3H), TFA salt |
| 47.9 | 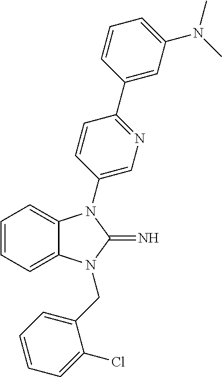 [SMILES: CN(C)c1cccc(-c2ccc(-n3c(=N)n(Cc4ccccc4Cl)c4ccccc43)cn2)c1] | (3-{5-[3-(2-Chloro- benzyl)-2-imino- 2,3-dihydro- benzoimidazol-1- yl]-pyridin-2-yl}- phenyl)-dimethyl- amine | 1.21 (A) | (M + 2 H)/2 227.5 | |

*t_{R} [min] (method);
**M + H (or specified);
***d⁶-dmso, except otherwise specified [ppm]

### Example 48

### N-(4-Chloro-2-{5-[2-imino-3-(1H-indol-4-ylmethyl)-2,3-dihydro-benzoimidazol-1-yl]-pyridin-2-yl}-phenyl)-acetamide

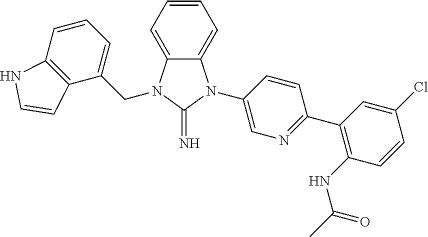 [SMILES: CC(=O)Nc1ccc(Cl)cc1-c1ccc(-n2c(=N)n(Cc3cccc4[nH]ccc34)c3ccccc32)cn1]

To a suspension of 78% pure N-{2-[5-(2-Amino-benzoimidazol-1-yl)-pyridin-2-yl]-4-chloro-phenyl}-acetamide (Step 1, 200.0 mg, assumed 0.41 mmol) in MeCN (2.64 mL) were added potassium iodide (88.0 mg, 0.53 mmol) and intermediate N (164.0 mg, 0.53 mmol). The resulting slurry was heated to 110° C. for 10 min under microwave irradiation. The medium was evaporated to dryness. The crude was taken up in saturated aqueous sodium bicarbonate solution (20 mL) and EtOAc (20 mL). The mixture was shaken and the phases were separated. The organic portion was dried over Na2SO4 and concentrated to a crude solid. The crude solid was dissolved in CH2Cl2 (0.32 mL) and TFA (0.16 mL) and the resulting mixture was stirred at rt for 30 minutes. The medium was then evaporated to dryness and purified by reverse-phase preparative HPLC (Method E, gradient from 10% MeCN (+0.1% TFA) in water (+0.1% TFA) to 70% over 14 minutes) yielding, after pooling and lyophilisation of product-containing fractions, the title compound as its TFA salt. The salt was dissolved in EtOAc (5 mL), washed with saturated aqueous sodium bicarbonate solution (5 mL), and the organic portion was finally dried over Na2SO4 and evaporated to furnish the title compound as a yellow solid, 29 mg (14%). HPLC/MS (Method D) t_{R} 2.45 minute, M+H 507.0-509.0. 1H NMR (Acetone-d₆) Ppm 2.17 (s, 3H) 5.52 (s, 2H) 6.81 (d, 1H) 6.90-7.17 (m, 7H) 7.31-7.52 (m, 3H) 7.91 (d, 1H) 8.23 (d, 1H) 8.33 (dd, 1H) 8.60 (d, 1H) 9.10 (d, 1H) 10.46 (br. s., 1H) 11.91 (br. s., 1H).

### Step 1: N-{2-[5-(2-Amino-benzoimidazol-1-yl)-pyridin-2-yl]-4-chloro-phenyl}-acetamide

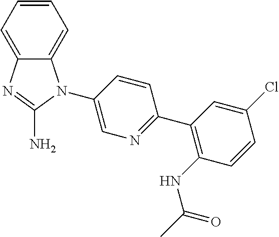 [SMILES: CC(=O)Nc1ccc(Cl)cc1-c1ccc(-n2c(N)nc3ccccc32)cn1]

A mixture composed of 1-(6-Chloro-pyridin-3-yl)-1H-benzoimidazol-2-ylamine (intermediate B, 500.0 mg, 2.04 mmol), 2-Acetamido-5-chlorophenylboronic acid pinacol ester (603.0 mg, 2.04 mmol), Pd(PPh₃)₄ (15.6 mg, 13.5 μmol) and aqueous Na₂CO₃ 2M (1100 μl, 2.20 mmol) in DME (10.2 mL) was heated to 150° C. for 17 min under microwave irradiation. The medium was diluted with MeOH (20 ml), filtered through diatomaceous earth and evaporated to dryness. The residue was taken up in EtOAc (200 mL) and washed with aqueous saturated sodium bicarbonate solution (2×100 mL) and brine (100 mL). The phases were separated, and the organic portion dried over Na2SO4 and concentrated to give the product as a dark oil, 625 mg, 78% UV diode array purity. The crude was used in the next step without further purification. HPLC/MS (Method A) t_{R} 1.05 minute (78%), M+H 378.0-380.0.

The following examples 48.1 to 48.2 were synthesized in a manner analogous to that used for the synthesis of example 48, from intermediate B and various aryl boronic acids or boronate esters (purchased or obtained from the corresponding aryl bromides as depicted in the synthesis of example 15).

| Ex. | structure | name | HPLC* | MS** | 1H NMR*** |
|---|---|---|---|---|---|
| 48.1 | 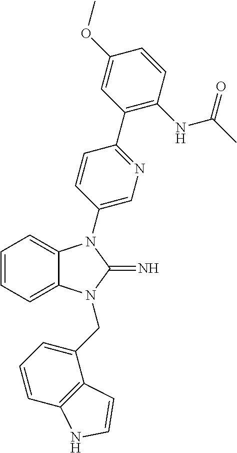 [SMILES: COc1ccc(NC(C)=O)c(-c2ccc(-n3c(=N)n(Cc4cccc5[nH]ccc45)c4ccccc43)cn2)c1] | N-(2-{5-[2- Imino-3-(1H- indol-4-ylmeth- yl)-2,3- dihydro- benzoimidazol- 1-yl]- pyridin-2-yl}- 4-methoxy- phenyl)- acetamide | 1.02 (A) | 503.0 | Ppm 2.09 (s, 3 H) 3.90 (s, 3 H) 5.85 (s, 2 H) 6.77 (d, 1 H) 6.95- 7.15 (m, 4 H) 7.16- 7.26 (m, 3 H) 7.27- 7.34 (m, 1 H) 7.38 (d, 1 H) 7.43-7.51 (m, 2 H) 8.19 (d, 1 H) 8.30- 8.40 (m, 2 H) 9.13 (d, 1 H) 10.60 (br. s., 1 H) 11.19 (br. s., 1 H). |
| 48.2 | 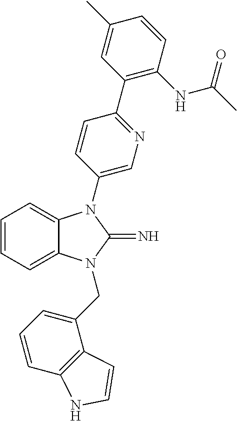 [SMILES: CC(=O)Nc1ccc(C)cc1-c1ccc(-n2c(=N)n(Cc3cccc4[nH]ccc34)c3ccccc32)cn1] | N-(2-{5-[2- Imino-3-(1H- indol-4-ylmeth- yl)-2,3- dihydro- benzoimidazol- 1-yl]- pyridin-2-yl}- 4-methyl- phenyl)-ace- tamide | 1.10 (A) | 487.1 | Ppm 2.14 (s, 3 H) 2.41 (s, 3 H) 5.50 (s, 2 H) 6.84 (d, 1 H) 6.88- 7.19 (m, 7 H) 7.22- 7.32 (m, 1 H) 7.35- 7.52 (m, 2 H) 7.73 (s, 1 H) 8.19 (d, 1 H) 8.29 (dd, 1 H) 8.47 (d, 1 H) 9.06 (d, 1 H) 10.42 (br. s., 1 H) 11.68 (br. s., 1 H). |

*t_{R} [min] (method);
**M + H (or specified);
***d⁶-acetone

### Example 49

### N-(2-{5-[3-(1H-Benzoimidazol-4-ylmethyl)-2-imino-2,3-dihydro-benzoimidazol-1-yl]-pyridin-2-yl}-4-chloro-phenyl)-acetamide

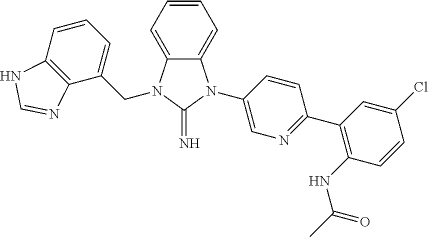 [SMILES: CC(=O)Nc1ccc(Cl)cc1-c1ccc(-n2c(=N)n(Cc3cccc4[nH]cnc34)c3ccccc32)cn1]

The title compound was synthesized in a manner analogous to that used for the synthesis of example 48, using intermediate AG instead of intermediate N. HPLC/MS (Method A) t_{R} 1.21 minute, M+H 508. ¹H NMR (Acetone-d₆) Ppm 2.10 (s, 3H) 5.48 (s, 2H) 6.86-7.09 (m, 3H) 7.12-7.34 (m, 3H) 7.48-7.58 (m, 3H) 7.59-7.67 (m, 1H) 7.89 (d, 1H) 8.12 (d, 1H) 8.19-8.28 (m, 2H) 8.33 (s, 1H) 9.00 (d, 1H) 11.29 (br. s., 1H).

### Example 50

### N-(4-Chloro-2-{5-[6-fluoro-2-imino-3-(1H-indol-4-ylmethyl)-2,3-dihydro-benzoimidazol-1-yl]-pyridin-2-yl}phenyl)-acetamide

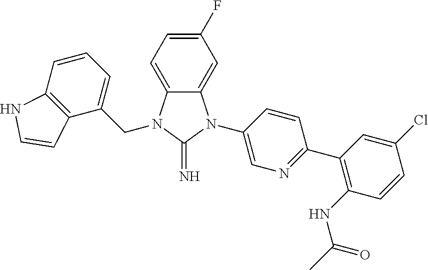 [SMILES: CC(=O)Nc1ccc(Cl)cc1-c1ccc(-n2c(=N)n(Cc3cccc4[nH]ccc34)c3ccc(F)cc32)cn1]

To a solution of N-{2-[5-(2-Amino-6-fluoro-benzoimidazol-1-yl)-pyridin-2-yl]-4-chloro-phenyl}-acetamide (Step 1, 100 mg, 0.253 mmol) in acetonitrile (505 μL) were added intermediate N (78 mg, 0.253 mmol) and KI (42 mg, 0.253 mmol). The resulting reaction mixture was stirred at 110° C. for 10 minutes under microwave irradiation. The reaction mixture was then evaporated to dryness taken up in AcOEt (10 mL) and extracted with saturated sodium bicarbonate solution (2×5 mL). The organic phase was dried over Na2SO4, filtered and concentrated to give a brown oil. This oil was treated with TFA (5 mL) for 5 minutes at rt, then quenched with water (10 mL). The medium was diluted with AcOEt (50 mL) and water (20 mL) and basified using solid NaHCO3. The phases were separated, the aqueous phase was washed with AcOEt (2×50 mL), the combined organics were dried over Na2SO4, filtered and evaporated to dryness. The crude product was purified by reverse-phase preparative HPLC (Method E, gradient from 20% (MeCN+0.1% TFA) in (Water+0.1% TFA) to 80% over 14 minutes). Product-containing fractions were pooled and evaporated to dryness. The residue was partitioned between AcOEt (10 mL) and saturated sodium bicarbonate solution (5 mL) to effect the removal of the TFA salt and furnish, after drying of the organic portion, the title compound as a white solid, 20 mg (15%). HPLC/MS (Method D) t_{R} 2.08 minute, M+H 525.0-527.0. 1H NMR (Acetone-d₆) Ppm 2.17 (s, 3H) 5.46 (s, 2H) 6.64-6.75 (m, 1H) 6.77-6.93 (m, 3H) 7.01-7.17 (m, 2H) 7.34-7.52 (m, 3H) 7.93 (d, 1H) 8.16-8.29 (m, 1H) 8.31-8.40 (m, 1H) 8.62 (d, 1H) 9.10 (d, 1H) 10.43 (br. s., 1H) 11.98 (br. s., 1H).

### Step 1: N-{2-[5-(2-Amino-6-fluoro-benzoimidazol-1-yl)-pyridin-2-yl]-4-chloro-phenyl}-acetamide

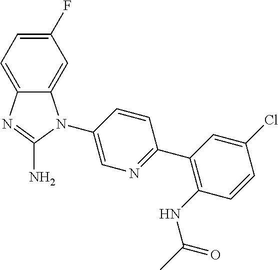 [SMILES: CC(=O)Nc1ccc(Cl)cc1-c1ccc(-n2c(N)nc3ccc(F)cc32)cn1]

A mixture composed of 1-(6-Chloro-pyridin-3-yl)-6-fluoro-1H-benzoimidazol-2-ylamine (intermediate C, 1.60 g, 6.09 mmol), 2-acetamido-5-chlorophenylboronic acid pinacol ester (1.80 g, 6.09 mmol), Pd(PPh3)2Cl2 (0.21 g, 0.305 mmol) and aqueous Na₂CO₃ 2M (15.2 mL, 30.5 mmol) in DME (30.5 mL) was heated to 150° C. for 16 min under microwave irradiation. The medium was diluted with EtOAc (100 mL), filtered through Florisil™ and washed with aqueous saturated sodium bicarbonate solution (2×100 mL). The organic phase was dried over Na2SO4 and concentrated to give the product as a dark oil. The oil was purified by chromatography on silica gel, using a 0% to 100% gradient of eluent B (EtOAc/MeOH/NH4OH:90/9/1) in eluent A (EtOAc), yielding the title product as a brown solid, 1.03 g (43%). HPLC/MS (Method A) t_{R} 1.01 minute, M+H 396.0-398.0. 1H NMR (DMSO-d₆) Ppm 2.11 (s, 3H) 6.51 (s, 2H) 6.78-6.95 (m, 2H) 7.22 (dd, 1H) 7.53 (dd, 1H) 7.90 (d, 1H) 8.16 (d, 2H) 8.28 (d, 1H) 8.90 (t, 1H) 11.35 (br. s., 1H).

The following examples 50.1 to 50.2 were synthesized in a manner analogous to that used for the synthesis of example 50, from intermediate C and various aryl boronic acids or boronate esters (purchased or obtained from the corresponding aryl bromides as depicted in the synthesis of example 15). The examples 50.3 to 50.6 were synthesized in a manner analogous to that used for the synthesis of example 50, from N-{2-[5-(2-Amino-6-fluoro-benzoimidazol-1-yl)-pyridin-2-yl]-4-chloro-phenyl}-acetamide (ex. 50, step 1) and the various alkyl bromide specified in the footnotes.

| Ex. | structure | name | HPLC* | MS** | 1H NMR*** |
|---|---|---|---|---|---|
| 50.1 | 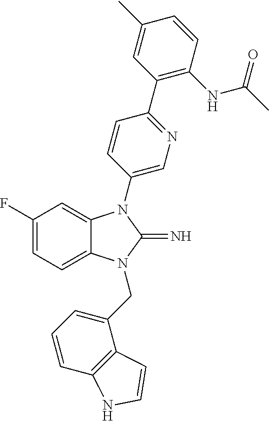 [SMILES: CC(=O)Nc1ccc(C)cc1-c1ccc(-n2c(=N)n(Cc3cccc4[nH]ccc34)c3ccc(F)cc32)cn1] | N-(2-{5-[6-Fluoro-2- imino-3-(1H-indol- 4-ylmethyl)-2,3- dihydro-benzoimida- zol-1-yl]-pyridin- 2-yl}-4-methyl- phenyl)-acetamide | 1.12 (A) | 505.1 | (Acetone-d₆) Ppm 2.14 (s, 3H) 2.41 (s, 3H) 5.45 (s, 2H) 5.75 (br. s., 1H) 6.61- 6.74 (m, 1H) 6.75- 6.93 (m, 3H) 7.02- 7.17 (m, 2H) 7.27 (dd, 1H) 7.33-7.49 (m, 2H) 7.73 (d, 1H) 8.10-8.22 (m, 1H) 8.28 (dd, 1H) 8.48 (d, 1H) 9.05 (d, 1H) 10.41 (br. s., 1H) 11.86 (br. s., 1H) |
| 50.2 | 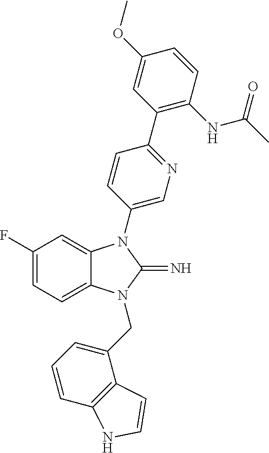 [SMILES: COc1ccc(NC(C)=O)c(-c2ccc(-n3c(=N)n(Cc4cccc5[nH]ccc45)c4ccc(F)cc43)cn2)c1] | N-(2-{5-[6-Fluoro-2- imino-3-(1H-indol- 4-ylmethyl)-2,3- dihydro-benzoimida- zol-1-yl]-pyridin- 2-yl}-4-methoxy- phenyl)-acetamide | 1.07 (A) | 521.0 | 2.05 (s, 3H) 3.84 (s, 3H) 5.44 (s, 2H) 6.70 (br. s., 1H) 6.74-6.92 (m, 3H) 6.96 (d, 1H) 7.01- 7.11 (m, 2H) 7.29- 7.45 (m, 4H) 7.95 (d, 1H) 8.04 (d, 1H) 8.22 (dd, 1H) 8.98 (d, 1H) 10.73 (br. s., 1H) 11.23 (br. s., 1H) |
| 50.3^{a} | 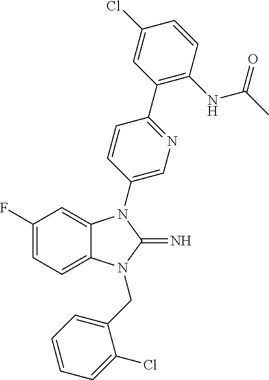 [SMILES: CC(=O)Nc1ccc(Cl)cc1-c1ccc(-n2c(=N)n(Cc3ccccc3Cl)c3ccc(F)cc32)cn1] | N-(4-Chloro-2-{5- [3-(2-chloro- benzyl)-6-fluoro-2- imino-2,3-dihydro- benzoimidazol-1- yl]-pyridin-2-yl}- phenyl)-acetamide | 1.30 (A) | 520- 522 | |
| 50.4^{b} | 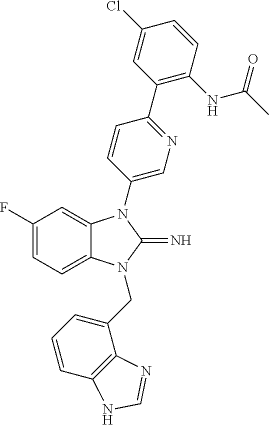 [SMILES: CC(=O)Nc1ccc(Cl)cc1-c1ccc(-n2c(=N)n(Cc3cccc4[nH]cnc34)c3ccc(F)cc32)cn1] | N-(2-{5-[3-(1H- Benzoimidazol-4- ylmeth- yl)-6-fluoro-2- imino-2,3-dihydro- benzoimidazol-1- yl]-pyridin-2-yl}-4- chloro-phenyl)- acetamide | 2.00 (D) | 526.0- 528.0 | 2.10 (s, 3H) 5.44 (br. s., 2H) 6.69- 6.95 (m, 2H) 6.96- 7.39 (m, 3H) 7.44- 7.64 (m, 3H) 7.88 (s, 1H) 8.11 (d, 1H) 8.23 (d, 2H) 8.32 (s, 1H) 8.98 (s, 1H) 11.29 (br. s., 1H) 12.61 (br. s., 1H) |
| 50.5^{c} | 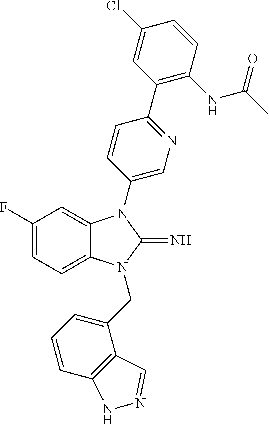 [SMILES: CC(=O)Nc1ccc(Cl)cc1-c1ccc(-n2c(=N)n(Cc3cccc4[nH]ncc34)c3ccc(F)cc32)cn1] | N-(4-Chloro-2-{5- [6-fluoro-2-imino- 3-(1H-indazol-4- ylmethyl)-2,3-dihydro- benzoimidazol- 1-yl]-pyridin-2-yl}- phenyl)- acetamide | 1.17 (A) | 526.1- 528.1 | 2.12 (s, 3H) 5.91 (s, 2H) 6.97 (d, 1H) 7.18-7.27 (m, 2H) 7.30-7.39 (m, 1H) 7.46 (dd, 1H) 7.53- 7.66 (m, 2H) 7.93 (d, 1H) 8.17-8.37 (m, 3H) 8.45 (dd, 1H) 9.02-9.30 (m, 3H) 11.22 (br. s., 1H) 13.33 (br. s., 1H) |
| 50.6^{d} | 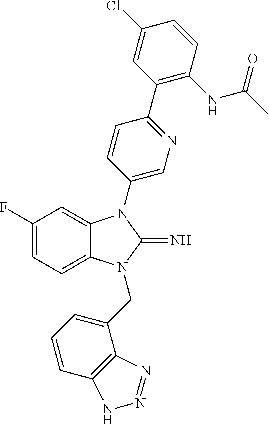 [SMILES: CC(=O)Nc1ccc(Cl)cc1-c1ccc(-n2c(=N)n(Cc3cccc4[nH]nnc34)c3ccc(F)cc32)cn1] | N-(2-{5-[3-(1H- Benzotriazol-4- ylmeth- yl)-6-fluoro-2- imino-2,3-dihydro- benzoimidazol-1-yl]- pyridin-2-yl}-4- chloro-phenyl)- acetamide | 1.92 (D) | 526.7- 528.8 | 2.11 (s, 3H) 5.60 (s, 2H) 6.81-6.94 (m, 2H) 7.15-7.20 (m, 2H) 7.31-7.44 (m, 2H) 7.53 (dd, 1H) 7.84 (d, 1H) 7.89 (d, 1H) 8.14 (d, 1H) 8.21-8.27 (m, 2H) 9.01 (d, 1H) 11.28 (br. s., 1H) |

*t_{R} [min] (method);
**M + H (or specified);
***d⁶-dmso [ppm], except if indicated otherwise;
^{a}Synthesized using 1-bromomethyl-2-chloro-benzene.
^{b}Synthesized using intermediate AG.
^{c}Synthesized using commercially available 4-Chloromethyl-1H-indazole (Anichem H11106, CAS 944898-78-6).
^{d}Synthesized using intermediate AH.

### Example 51

### N-(4′-{3-(2-Chloro-benzyl)-2-[(E,Z)-methylimino]-2,3-dihydro-benzoimidazol-1-yl}-biphenyl-2-yl)-acetamide

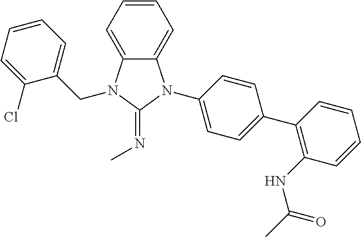 [SMILES: C/N=c1\n(Cc2ccccc2Cl)c2ccccc2n1-c1ccc(-c2ccccc2NC(C)=O)cc1]

To a solution of intermediate M (79 mg, 0.191 mmol) in MeCN was added methyl iodide (71.8 μL, 1.149 mmol). The resulting solution was stirred at 80° C. for 2 hrs. The medium was then allowed to cool and was evaporated to dryness. The medium was taken up in CH₂Cl₂ (5 mL) and washed with saturated sodium bicarbonate solution (5 mL). The aqueous phase was extracted further with CH₂Cl₂ (2×2 ml) and the combined organics were dried over Na2SO4 and concentrated to a crude oil. The oil was taken up in DME (1.5 mL) and 2-acetylaminophenylboronic acid (41.2 mg, 0.230 mmol), Pd(PPh3)4 (11.09 mg, 9.60 μmol) and aqueous Na2CO3 2M (0.480 ml, 0.960 mmol) were successively added. The resulting mixture was heated to 150° C. for 15 min under microwave irradiation. The medium was then diluted with EtOAc (5 mL), and filtered through consecutive plugs of Florisil™ and Na₂SO₄. The plugs were washed with EtOAc (2×2 mL) and the combined organics were concentrated to a brown oil. The oil was purified by chromatography on silica gel, using a 20% to 100% gradient of eluent B (EtOAc/NH4OH:98/2) in eluent A (CH₂Cl₂/Heptane:1/1), yielding the title product as a white solid, 14 mg (15%). HPLC/MS (Method D) t_{R} 2.21 minute, M+H 481.0-483.1. 1H NMR (DMSO-d6) Ppm 1.86 (s, 3H) 2.60 (br. s., 3H) 5.14 (br. s., 2H) 6.64 (d, 1H) 6.81 (d, 1H) 6.87 (t, 1H) 6.94 (t, 1H) 7.08 (d, 1H) 7.29-7.37 (m, 3H) 7.40 (t, 1H) 7.42-7.45 (m, 2H) 7.52-7.57 (m, 3H) 7.61 (br. S., 2H) 9.41 (s, 1H).

The following examples 51.1 to 51.3 were synthesized in a manner analogous to that used for the synthesis of example 51, from intermediate M and various commercially available alkyl iodides or bromides.

| Ex. | structure | name | HPLC** | MS*** |
|---|---|---|---|---|
| 51.1 | 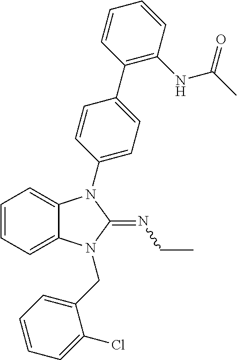 [SMILES: CCN=c1n(Cc2ccccc2Cl)c2ccccc2n1-c1ccc(-c2ccccc2NC(C)=O)cc1] | N-{4′-[3-(2-Chloro- benzyl)-2-ethyl- imino-2,3-dihydro- benzoimidazol-1-yl]- biphenyl-2-yl}- acetamide | 2.29 (D) | 495.1- 497.1 |
| 51.2 | 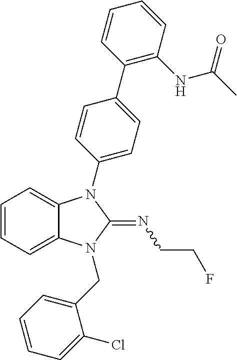 [SMILES: CC(=O)Nc1ccccc1-c1ccc(-n2c(=NCCF)n(Cc3ccccc3Cl)c3ccccc32)cc1] | N-{4′-[3-(2-Chloro- benzyl)-2-(2-fluoro- ethylimino)-2,3- dihydro-benzoimi- dazol-1-yl]- biphenyl-2-yl}- acetamide | 2.14 (D) | 513.1- 515.1 |
| 51.3^{a} | 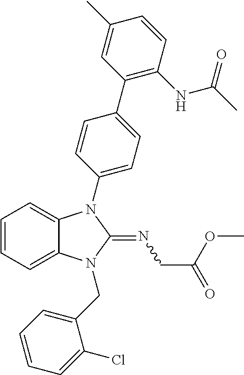 [SMILES: COC(=O)CN=c1n(Cc2ccccc2Cl)c2ccccc2n1-c1ccc(-c2cc(C)ccc2NC(C)=O)cc1] | [1-(2′-Acetylamino- 5′-methyl-biphenyl- 4-yl)-3-(2-chloro- benzyl)-1,3-di hydro- benzoimidazol-2- ylideneamino]- acetic acid methyl ester | 1.23 (A) | 553.0- 555.0 |

*t_{R} [min] (method);
**M + H (or specified);
^{a}Synthesized using intermediate X instead of 2-aceylaminophenylboronic acid.

### Example 52

### N N-{5-Chloro-4′-[2-cyclopropylimino-3-(1H-indol-4-ylmethyl)-2,3-dihydro-benzoimidazol-1-yl]-biphenyl-2-yl}-acetamide

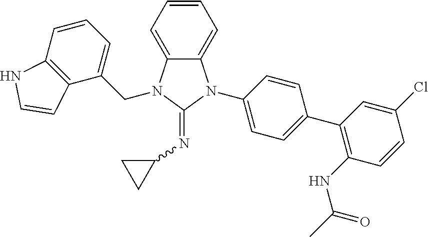 [SMILES: CC(=O)Nc1ccc(Cl)cc1-c1ccc(-n2c(=NC3CC3)n(Cc3cccc4[nH]ccc34)c3ccccc32)cc1]

To a solution of N-[5-Chloro-4′-(2-cyclopropylamino-benzoimidazol-1-yl)-biphenyl-2-yl]-acetamide (step 1, 39 mg, 0.094 mmol) in MeCN was added intermediate N (29.0 mg, 0.094 mmol) followed by KI (15.53 mg, 0.094 mmol). The resulting clear solution was stirred at 110° C. for 10 min. The medium was evaporated to dryness and the resulting glass treated with TFA (1000 μL, 12.98 mmol) for 10 minutes. After that time, the medium was evaporated to dryness. The crude product was purified by reverse-phase preparative HPLC (Method E, gradient from 20% (MeCN+0.1% TFA) in (Water+0.1% TFA) to 80% over 14 minutes). Product-containing fractions were pooled and evaporated to dryness to furnish the title product as its TFA salt. The product was liberated from its salt by partitioning between CH2Cl2 and saturated aqueous sodium bicarbonate solution, finally leading to the freetitle compound, 9 mg (18%). HPLC/MS (Method D) t_{R} 2.30 minute, M+H 546.0.

### Step 1: N-[5-Chloro-4′-(2-cyclopropylamino-benzoimidazol-1-yl)-biphenyl-2-yl]-acetamide

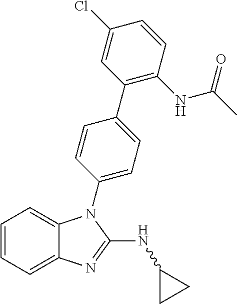 [SMILES: CC(=O)Nc1ccc(Cl)cc1-c1ccc(-n2c(NC3CC3)nc3ccccc32)cc1]

A mixture composed of [1-(4-Bromo-phenyl)-1H-benzoimidazol-2-yl]-cyclopropyl-amine (step 2, 50 mg, 0.152 mmol), 2-acetamido-5-chlorophenyl boronic acid, pinacol ester (45.0 mg, 0.152 mmol), Pd(PPh3)2Cl2 (5.35 mg, 7.62 μmol) and aqueous Na2CO3 2M (381 μL, 0.762 mmol) in DME (762 μL) was heated to 150° C. for 16 min under microwave irradiation. The medium was diluted with EtOAc (5 mL), and filtered through consecutive plugs of Florisil™ and Na₂SO₄. The plugs were washed with EtOAc (2×2 mL) and the combined organics were concentrated to a brown oil. The oil was purified by chromatography on silica gel, using a 0% to 100% gradient of eluent B (EtOAc/MeOH:8/2) in eluent A (EtOAc), yielding the title product as a colorless resin, 39 mg (61%). HPLC/MS (Method A) t_{R} 1.08 minute, M+H 417.1-419.1.

### Step 2: [1-(4-Bromo-phenyl)-1H-benzoimidazol-2-yl]-cyclopropyl-amine

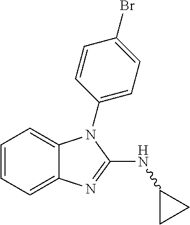 [SMILES: Brc1ccc(-n2c(NC3CC3)nc3ccccc32)cc1]

To a solution of 1-(4-Bromo-phenyl)-2-chloro-1H-benzoimidazole (step 3, 150 mg, 0.488 mmol) in 1.25 M HCl in iPrOH (2.5 ml) was added cyclopropanamine (338 μL, 4.88 mmol). The resulting clear solution was stirred at 150° C. for 120 min under microwave irradiation. The medium was then evaporated to a crude oil. The crude was taken up in EtOAc (20 mL) and washed with saturated aqueous sodium bicarbonate solution (2×15 mL). The combined organics were dried over Na2SO4 and concentrated to a crude residue which was purified by chromatography on silica gel, using a 5% to 50% gradient of eluent B (EtOAc) in eluent A (CH₂Cl₂/Heptane:1/1), yielding the title product as a white solid, 157 mg (98%). HPLC/MS (Method A) t_{R} 1.07 minute, M+H 327.9-329.9.

### Step 3: 1-(4-Bromo-phenyl)-2-chloro-1H-benzoimidazole

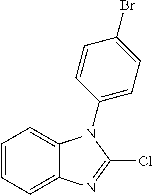 [SMILES: Clc1nc2ccccc2n1-c1ccc(Br)cc1]

A solution of 1-(4-Bromo-phenyl)-1,3-dihydro-benzoimidazol-2-one (Step 4, 700 mg, 2.421 mmol) in POCl₃ (8 mL) was stirred at reflux for 3 hrs. The medium was allowed to cool down to rt. The mixture was then very slowly poured into well-stirred saturated aqueous sodium bicarbonate solution (200 mL), adding more solid NaHCO3 to keep the pH basic. The aqueous suspension was then extracted with EtOAc (3×40 mL) and the combined organics dried over Na2SO4 and concentrated to a crude brown oil. The oil was purified by chromatography on silica gel, using a 0% to 10% gradient of eluent B (EtOAc) in eluent A (CH₂Cl₂/Heptane:1/1), yielding the title product as a white solid, 571 mg (76%). HPLC/MS (Method A) t_{R} 1.60 minute, M+H 306.9-308.9. 1H NMR (CDCl₃) Ppm 7.13 (d, 1H) 7.30 (m, 4H) 7.74 (m, 3H)

### Step 4: 1-(4-Bromo-phenyl)-1,3-dihydro-benzoimidazol-2-one

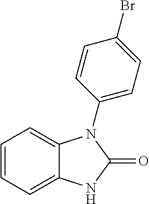 [SMILES: O=c1[nH]c2ccccc2n1-c1ccc(Br)cc1]

To a solution of N1-(4-bromophenyl)benzene-1,2-diamine (intermediate A, step A1, 702 mg, 2.67 mmol) in CH2Cl2 (8.9 mL) was added carbonyldiimidazole (464 mg, 2.86 mmol). The resulting brown solution was stirred at reflux for 60 min. The medium was then evaporated to dryness. The crude was taken up in EtOAc (75 mL) and washed with 1M aqueous HCl (3×20 mL). The organics were dried over Na2SO4 and concentrated to a crude solid, 739 mg, 91%, 95% UV purity. HPLC/MS (Method A) t_{R} 1.39 minute, M+H 289-291.

### Example 53

### N-{5-Chloro-4′-[3-(1H-indol-4-ylmethyl)-2-isopropylimino-2,3-dihydrobenzoimidazol-1-yl]-biphenyl-2-yl}-acetamide

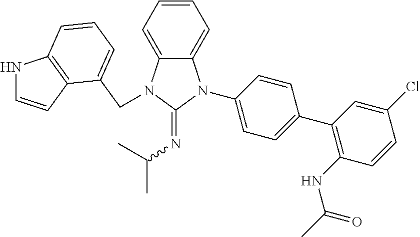 [SMILES: CC(=O)Nc1ccc(Cl)cc1-c1ccc(-n2c(=NC(C)C)n(Cc3cccc4[nH]ccc34)c3ccccc32)cc1]

The title compound was synthesized in a manner analogous to that used for the synthesis of example 52, using isopropylamine instead of cyclopropanamine in step 2. HPLC/MS (Method A) t_{R} 1.38 minute, M+H 548.1-550.1.

### Example 68

### N-{4′-[3-(2-Fluoro-benzyl)-2-imino-2,3-dihydro-benzoimidazol-1-yl]-biphenyl-2-yl}-acetamide

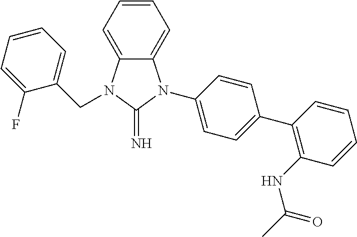 [SMILES: CC(=O)Nc1ccccc1-c1ccc(-n2c(=N)n(Cc3ccccc3F)c3ccccc32)cc1]

To a suspension of intermediate L (42.4 mg, 0.124 mmol) in MeCN (1.2 mL) were added potassium iodide (21.0 mg, 0.124 mmol) and 2-fluorobenzyl bromide (15.0 μL, 0.124 mmol). The resulting slurry was heated to 110° C. for 8 minutes under microwave irradiation.

The medium was then diluted with water (5 mL) and extracted with EtOAc (3×5 mL). The combined organic extracts were evaporated to dryness. The crude residue was purified by reverse-phase preparative HPLC (Method E, gradient elution of (MeCN+0.1% TFA) in (Water+0.1% TFA)). Product-containing fractions were pooled and evaporated to dryness to furnish the title compound as its TFA salt, 20.3 mg (29%). HPLC/MS (method A) t_{R} 1.13 minute, M+H 451.0.

The following examples 68.1 to 68.3 were synthesized in a manner analogous to that used for the synthesis of example 68, using commercially available benzyl bromides or intermediate AG.

| Ex. | structure | name | HPLC | MS** | 1H NMR |
|---|---|---|---|---|---|
| 68.1 | 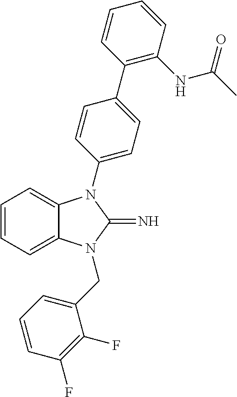 [SMILES: CC(=O)Nc1ccccc1-c1ccc(-n2c(=N)n(Cc3cccc(F)c3F)c3ccccc32)cc1] | N-{4′-[3-(2,3- Difluoro-benzyl)-2- imino-2,3-dihydro- benzoimidazol-1-yl]- biphenyl-2-yl}- acetamide | 1.15 (A) | 469.0 | |
| 68.2 | 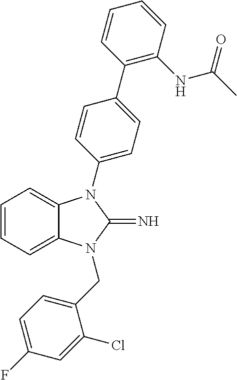 [SMILES: CC(=O)Nc1ccccc1-c1ccc(-n2c(=N)n(Cc3ccc(F)cc3Cl)c3ccccc32)cc1] | N-{4′-[3-(2-Chloro- 4-fluoro-benzyl)- 2-imino-2,3- dihydro- benzoimidazol- 1-yl]-biphenyl-2-yl}- acetamide | 1.19 (A) | 484.9- 486.9 | |
| 68.3^{a} | 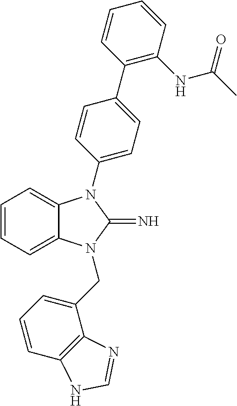 [SMILES: CC(=O)Nc1ccccc1-c1ccc(-n2c(=N)n(Cc3cccc4[nH]cnc34)c3ccccc32)cc1] | N-{4′-[3-(1H- Benzoimidazol-4- ylmethyl)-2-imino- 2,3-dihydro- benzoimidazol-1- yl]-biphenyl-2-yl}- acetamide | 1.06 (A) | 473.1 | (DMSO-d₆) Ppm 1.95 (s, 3H) 5.84 (s, 2H) 7.08 (m, 1H) 7.30-7.47 (m, 7H) 7.57 (d, 1H) 7.64-7.82 (m, 6H) 8.69 (bs, 1H) 9.12 (bs, 2H) 9.30 (s, 1H) |

*t_{R} [min] (method);
**M + H (or specified);
^{a}Heated at 110° C. for 20 minutes instead of 8, thus effecting the removal of the Boc protecting group.

### Example 69

### N-{5-Chloro-4′-[6-fluoro-2-imino-3-(1H-indol-4-ylmethyl)-2,3-dihydro-benzoimidazol-1-yl]biphenyl-2-yl}-acetamide

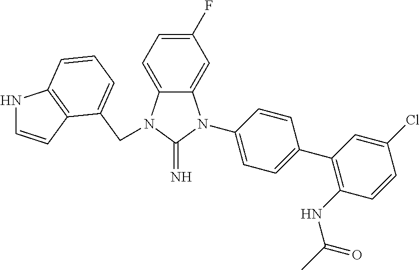 [SMILES: CC(=O)Nc1ccc(Cl)cc1-c1ccc(-n2c(=N)n(Cc3cccc4[nH]ccc34)c3ccc(F)cc32)cc1]

A solution of crude 4-[3-(2′-Acetylamino-5′-chloro-biphenyl-4-yl)-5-fluoro-2-imino-2,3-dihydro-benzoimidazol-1-ylmethyl]-indole-1-carboxylic acid tert-butyl ester (step 1, 70 mg, 0.112 mmol) and TFA (277 μL, 3.59 mmol) in CH₂Cl₂ (1122 μL) was stirred for 30 min at rt. The reaction mixture then quenched with water (5 mL) and diluted with CH₂Cl₂ (10 mL). The phases were separated and the aqueous layer was extracted with CH₂Cl₂ (2×5 mL). The combined organic portions were dried over Na₂SO₄, filtered and concentrated to a brown residue. The residue was purified by two consecutive chromatographies on silica gel, using a 5% to 100% gradient of eluent B (EtOAc/MeOH/NH4OH:89/9/1) in eluent A (heptane), yielding the title product as a solid, 20 mg (33%). HPLC/MS (Method A) tR 1.44 minute, M+H 524.1. ¹H NMR (dimethylsulfoxide-d6) Ppm 1.96 (br. s., 3H) 5.40 (s, 2H) 6.58-6.78 (m, 3H) 6.84 (ddd, 1H) 6.91-7.01 (m, 1H) 7.01-7.12 (m, 1H) 7.29-7.41 (m, 2H) 7.43-7.50 (m, 1H) 7.51-7.71 (m, 6H) 7.81 (d, 1H) 9.39 (br. s., 1H) 11.21 (br. s., 1H)

### Step 1: 4-[3-(2′-Acetylamino-5′-chloro-biphenyl-4-yl)-5-fluoro-2-imino-2,3-dihydrobenzoimidazol-1-ylmethyl]-indole-1-carboxylic acid tert-butyl ester

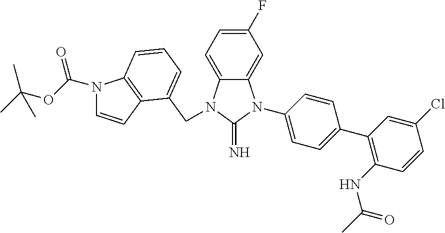 [SMILES: CC(=O)Nc1ccc(Cl)cc1-c1ccc(-n2c(=N)n(Cc3cccc4c3ccn4C(=O)OC(C)(C)C)c3ccc(F)cc32)cc1]

A suspension of N-[4′-(2-Amino-6-fluoro-benzoimidazol-1-yl)-5-chloro-biphenyl-2-yl]-acetamide (step 2, 90.0 mg, 0.228 mmol assumed for the purpose of calculations), intermediate N (71.0 mg, 0.228 mmol), and KI (37.8 mg, 0.228 mmol) in acetonitrile (456 μl) was stirred at 110° C. for 10 minutes under microwave irradiation. The reaction mixture was then concentrated under reduced pressure. The crude was dissolved in AcOEt (10 mL) and washed with saturated aqueous sodium bicarbonate solution (2×5 mL). The organic phase was dried Na₂SO₄, filtered and concentrated to give the crude title product as brown oil, 70 mg, which was directly taken to the next step.

### Step 2: N-[4′-(2-Amino-6-fluoro-benzoimidazol-1-yl)-5-chloro-biphenyl-2-yl]-acetamide

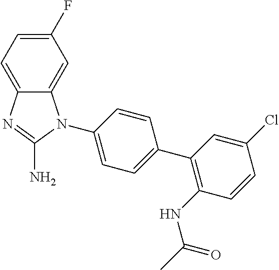 [SMILES: CC(=O)Nc1ccc(Cl)cc1-c1ccc(-n2c(N)nc3ccc(F)cc32)cc1]

To a suspension of intermediate H (500 mg, 1.63 mmol) in DME (5.4 mL) and Na2CO3 solution (2M in water) (2.72 mL) were added 2-acetamido-5-chlorophenylboronic acid, pinacol ester (531 mg, 1.79 mmol) and Pd(PPh₃)₄ (57 mg, 0.049 mmol). The resulting mixture was stirred for 17 minutes at 150° C. under microwave irradiation. The reaction mixture was then diluted with ACOEt (50 mL) and extracted with saturated aqueous sodium bicarbonate solution (2×20 mL). The organic phase was dried over Na₂SO₄, filtered and concentrated to give the title product as a brown solid (750 mg, 71% purity) which was used without further purification. HPLC/MS (method A) t_{R} 1.08 minute (71% UV diode array purity), M+H 395.0-397.0.

### Example 70

### N-{4′-[3-(1H-benzoimidazol-4-ylmethyl)-6-fluoro-2-imino-2,3-dihydro-benzoimidazol-1-yl]-5-chloro-biphenyl-2-yl}-acetamide

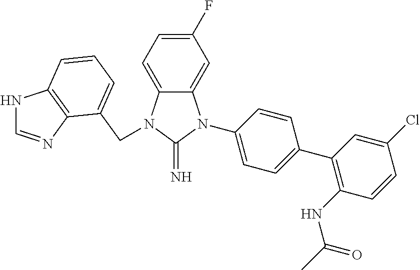 [SMILES: CC(=O)Nc1ccc(Cl)cc1-c1ccc(-n2c(=N)n(Cc3cccc4[nH]cnc34)c3ccc(F)cc32)cc1]

A suspension of N-[4′-(2-Amino-6-fluoro-benzoimidazol-1-yl)-5-chloro-biphenyl-2-yl]-acetamide (example 69 step 2, 40.0 mg, 0.101 mmol assumed for the purpose of calculations), intermediate AG (31.5 mg, 0.101 mmol), and KI (16.8 mg, 0.101 mmol) in acetonitrile (507 μL) was stirred at 110° C. for 10 minutes under microwave irradiation. The reaction mixture was then concentrated under reduced pressure. The crude was dissolved in AcOEt (50 mL) and washed with saturated aqueous sodium bicarbonate solution (2×20 mL). The organic phase was concentrated and the residue was treated with TFA (2 mL) for 5 minutes at rt. Water (10 mL) was added, followed by CH₂Cl₂ (30 mL). The medium was carefully basified by slow addition of sodium bicarbonate and the phases were separated. The aqueous layer was extracted with CH₂Cl₂ (3×20 mL) and the combined organic phases were dried over Na₂SO₄ and concentrated to a brown oil. The residue was purified by chromatography on silica gel, using a 10% to 100% gradient of eluent B (EtOAc/MeOH/NH4OH:50/47.5/2.5) in eluent A (heptane), yielding the title product as a solid, 13.6 mg (26%). HPLC/MS (Method D) t_{R} 1.95 minute, M+H 525.0-527.0. ¹H NMR (Dimethylsulfoxide-d₆) Ppm 1.96 (s, 3H) 5.53 (s, 2H) 6.73 (dd, 1H) 6.84-7.00 (m, 1H) 7.20 (t, 1H) 7.31 (br. s., 2H) 7.42-7.50 (m, 2H) 7.56 (d, 1H) 7.60-7.73 (m, 5H) 8.33 (s, 1H) 9.36 (s, 1H).

### Example 71

### N-{5-Chloro-4′-[3-(2-chloro-benzyl)-6-fluoro-2-imino-2,3-dihydro-benzoimidazol-1-yl]-biphenyl-2-yl}-acetamide

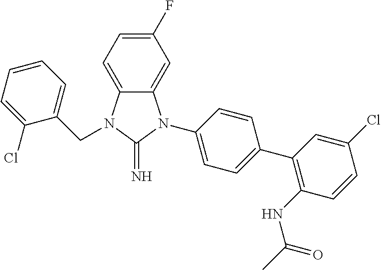 [SMILES: CC(=O)Nc1ccc(Cl)cc1-c1ccc(-n2c(=N)n(Cc3ccccc3Cl)c3ccc(F)cc32)cc1]

To a suspension of 3-(4-Bromo-phenyl)-1-(2-chloro-benzyl)-5-fluoro-1,3-dihydro-benzoimidazol-2-ylideneamine (step 1, 20.0 mg, 0.046 mmol) in DME (155 μL) and Na2CO3 solution (2M in water) (77 μL) were added 2-acetamido-5-chlorophenylboronic acid, pinacol ester (15.1 mg, 0.051 mmol) and Pd(PPh₃)₄ (1.6 mg, 1.4 μmol). The resulting mixture was stirred for 17 minutes at 150° C. under microwave irradiation. The reaction mixture was then diluted with ACOEt (5 mL) and extracted with saturated aqueous sodium bicarbonate solution (2×2 mL). The organic phase was dried over Na₂SO₄, filtered and concentrated. The crude residue was purifled by reverse-phase preparative HPLC (Method E, gradient elution of (MeCN+0.1% TFA) in (Water+0.1% TFA) from 0% to 100%). Product-containing fractions were pooled and evaporated to dryness to furnish the title compound as its TFA salt, 7.0 mg (22%). HPLC/MS (method A) t_{R} 1.34 minute, M+H 518.9-521.0.

### Step 1: 3-(4-Bromo-phenyl)-1-(2-chloro-benzyl)-5-fluoro-1,3-dihydro-benzoimidazol-2-ylideneamine

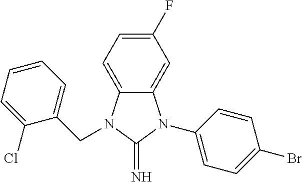 [SMILES: N=c1n(Cc2ccccc2Cl)c2ccc(F)cc2n1-c1ccc(Br)cc1]

A suspension of intermediate H (472 mg, 1.54 mmol), 2-chlorobenzyl bromide (200 μL, 1.54 mmol), and KI (258 mg, 1.54 mmol) in acetonitrile (3.0 mL) was stirred at 110° C. for 10 minutes under microwave irradiation. The reaction mixture was then concentrated under reduced pressure. The crude was partitioned between CH₂Cl₂ (100 mL) and water (50 mL), the phases were separated and the aqueous portion was extracted further with CH₂Cl₂ (2×50 mL). The combined organic layers were dried over Na₂SO₄ and concentrated. The residue was purified by chromatography on silica gel, using a 10% to 100% gradient of eluent B (EtOAc/7N NH3 in MeOH:99/1) in eluent A (heptane), yielding the title product as a solid, 339 mg (51%). HPLC/MS (Method A) t_{R} 1.25 minute, M+H 431.

### Example 72

### N-{5-Chloro-4′-[3-(2-chloro-benzyl)-2-imino-5-methyl-2,3-dihydro-benzoimidazol-1-yl]-biphenyl-2-yl}-acetamide

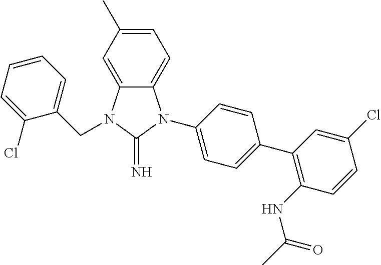 [SMILES: CC(=O)Nc1ccc(Cl)cc1-c1ccc(-n2c(=N)n(Cc3ccccc3Cl)c3cc(C)ccc32)cc1]

The title compound was synthesized in a manner analogous to that used for the synthesis of example 71, using intermediate AK instead of intermediate H in step 1. HPLC/MS (Method A) t_{R} 1.40 minute, M+H 515.0-517.0.

### Example 73

### N-{5-Chloro-4′-[7-chloro-2-imino-3-(1H-indol-4-ylmethyl)-2,3-dihydro-benzoimidazol-1-yl]-biphenyl-2-yl}-acetamide

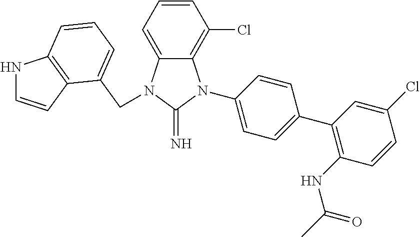 [SMILES: CC(=O)Nc1ccc(Cl)cc1-c1ccc(-n2c(=N)n(Cc3cccc4[nH]ccc34)c3cccc(Cl)c32)cc1]

N-[4′-(2-Amino-7-chloro-benzoimidazol-1-yl)-5-chloro-biphenyl-2-yl]-acetamide (step 1, 78 mg, 0.190 mmol) was dissolved in MeCN (2000 μl) and intermediate N (58.8 mg, 0.190 mmol) was added followed by KI (31.5 mg, 0.190 mmol). The resulting reaction mixture was stirred for 10 minutes at 110° C. under microwave irradiation. The reaction mixture was cooled to rt, diluted with CH₂Cl₂ (10 mL) and washed with 1N aqueous sodium hydroxide solution (3×5 mL) and brine (10 mL). The organic phase was then dried over Na2SO4, filtered and evaporated to dryness. The residue was purified by chromatography on silica gel, using a 30% to 100% gradient of eluent B (EtOAc+1% NH4OH) in eluent A (heptane/CH₂Cl₂: 1/1), yielding the Boc-protected title product as a solid, 26 mg. The solid was dissolved in CH₂Cl₂ (700 μl) and TFA (300 μl) was added to effect the Boc deprotection. The resulting reaction mixture was stirred for 1 hour at rt before evaporation to dyness. The resulting residue was diluted with CH₂Cl₂ (5 mL) and washed with saturated aqueous sodium bicarbonate solution (5 mL). The organic phase was dried over Na2SO4, filtered and evaporated to dryness. The residue was purified by chromatography on silica gel, using a 70% to 100% gradient of eluent B (EtOAc+1% NH4OH) in eluent A (heptane/CH₂Cl₂:1/1), finally yielding the title product as a solid, 14 mg (14%). HPLC/MS (Method D) t_{R} 1.06 minute, M+H 539.8-541.8. ¹H NMR (Dimethylsulfoxide-d6) Ppm 1.88 (s, 3H) 5.42 (s, 2H) 6.70-7.60 (m, 16H) 6.40 (s, 1H) 11.23 (s, 1H)

### Step 1: N-[4′-(2-Amino-7-chloro-benzoimidazol-1-yl)-5-chloro-biphenyl-2-yl]-acetamide

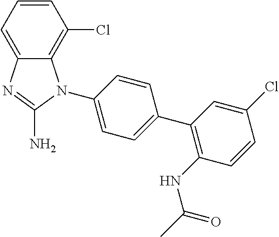 [SMILES: CC(=O)Nc1ccc(Cl)cc1-c1ccc(-n2c(N)nc3cccc(Cl)c32)cc1]

To a solution of intermediate AL (120 mg, 0.372 mmol) in DME (1.9 mL) were added 2-acetamido-5-chlorophenyl boronic acid pinacol ester (121 mg, 0.409 mmol), Pd(PPh3)4 (21.5 mg, 0.019 mmol) and Na2CO3 2M in water (0.93 mL, 1.860 mmol). The resulting reaction mixture was stirred at 150° C. for 17 minutes under microwave irradiation. The reaction mixture was then cooled down and filtered through diatomaceous earth which was subsequently washed with EtOAc (3×4 mL). The combined organic phases were then evaporated to dryness. The residue was purified by chromatography on amino-derivatized silica gel (Biotage cartridge 25+S KP-NH), using a 0% to 100% gradient of eluent B (EtOAc) in eluent A (heptane), yielding the title product as a solid, 165 mg (97%). HPLC/MS (Method A) t_{R} 1.06 minute, M+H 411.0-413.0.

### Example 74

### N-(4-Chloro-2-{5-[2-imino-3-(1H-indol-4-ylmethyl)-6-methoxy-2,3-dihydro-benzoimidazol-1-yl]-pyridin-2-yl}-phenyl)-acetamide

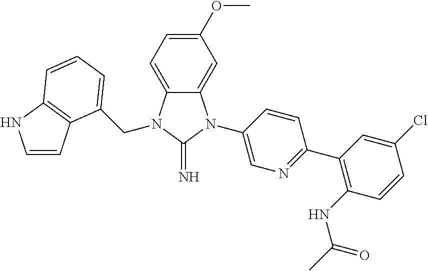 [SMILES: COc1ccc2c(c1)n(-c1ccc(-c3cc(Cl)ccc3NC(C)=O)nc1)c(=N)n2Cc1cccc2[nH]ccc12]

The title compound was synthesized in a manner analogous to that used for the synthesis of example 73, using intermediate AM instead of intermediate AL in step 1. HPLC/MS (Method D) t_{R} 2.13 minute, M+H 537.1-539.2. ¹H NMR (Dimethylsulfoxide-d6) Ppm 2.11 (s, 3H) 3.67 (s, 3H) 5.45 (br. s., 2H) 6.60 (br. s., 2H) 6.69 (br. s., 1H) 6.88 (d, 1H) 6.94 (d, 1H) 7.06 (t, 1H) 7.29-7.44 (m, 2H) 7.53 (dd, 1H) 7.90 (d, 1H) 8.16 (d, 1H) 8.21-8.33 (m, 2H) 9.03 (d, 1H) 11.12-11.39 (m, 2H).

### Example 75

### N-(4-Chloro-2-{5-[3-(2-chloro-benzyl)-2-imino-6-methoxy-2,3-dihydro-benzoimidazol-1-yl]-pyridin-2-yl}-phenyl)-acetamide

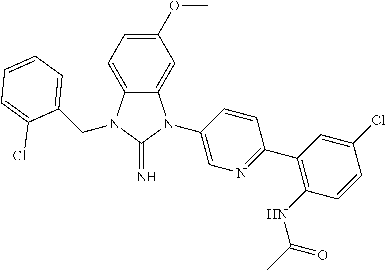 [SMILES: COc1ccc2c(c1)n(-c1ccc(-c3cc(Cl)ccc3NC(C)=O)nc1)c(=N)n2Cc1ccccc1Cl]

The title compound was synthesized in a manner analogous to that used for the synthesis of example 73, using intermediate AM instead of intermediate AL in step 1 and 2-chlorobenzyl bromide instead of intermediate N. The Boc-deprotection step was omitted. HPLC/MS (Method D) t_{R} 2.25 minute, M+H 532.1-534.1. ¹H NMR (Dimethylsulfoxide-d6) Ppm 2.11 (s, 3H) 3.74 (s, 3H) 5.56 (s, 2H) 6.79 (d, 1H) 7.19 (d, 1H) 7.31-7.39 (m, 2H) 7.43 (td, 1H) 7.51-7.68 (m, 2H) 7.94 (d, 1H) 8.16-8.34 (m, 2H) 8.41 (dd, 1H) 8.97 (s, 2H) 9.11 (d, 1H) 11.22 (br. s., 1H).

### Example 76

### N-{4′-[3-(1H-benzotriazol-4-ylmethyl)-2-imino-2,3-dihydro-benzoimidazol-1-yl]-5-chloro-biphenyl-2-yl}-acetamide

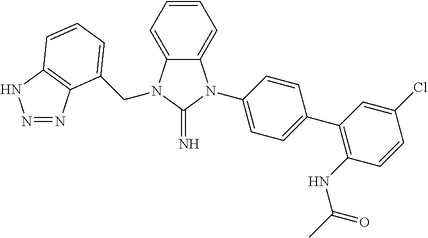 [SMILES: CC(=O)Nc1ccc(Cl)cc1-c1ccc(-n2c(=N)n(Cc3cccc4[nH]nnc34)c3ccccc32)cc1]

N-[4′-(2-Amino-benzoimidazol-1-yl)-5-chloro-biphenyl-2-yl]-acetamide (step 1, 60 mg, 0.159 mmol) was dissolved in MeCN (1.6 mL). Intermediate AH (49.7 mg, 0.159 mmol) was added, followed by KI (26.4 mg, 0.159 mmol).The resulting reaction mixture was stirred for 10 minutes at 110° C. under microwave irradiation. The medium was allowed to cool down to rt. The solids were removed by filtration, the cake was washed with CH₂Cl₂ (2×2 mL) and MeOH (2×2 mL) and the combined filtrates were evaporated to dryness. The crude residue was purified by reverse-phase preparative HPLC (Method E, gradient from 35% (MeOH/MeCN 3/1+0.1% TFA) in (Water+0.1% TFA) to 65% over 14 minutes). Product-containing fractions were pooled and evaporated to dryness. The residue was taken up in MeOH (1 mL) and desalted through a MP-carbonate resin cartridge (Polymer Labs). Final evaporation and drying afforded the pure title compound as a solid, 8 mg (10%). HPLC/MS (Method D) t_{R} 2.03 minute, M+H 508.0-510.0. ¹H NMR (Dimethylsulfoxide-d6) Ppm 1.96 (s, 3H) 5.58 (s, 2H) 6.90 (d, 1H) 7.05 (dt, 2H) 7.27 (t, 1H) 7.37 (d, 1H) 7.47 (dd, 2H) 7.59-7.71 (m, 6H) 7.81 (d, 1H) 9.39 (s, 1H).

### Step 1: N-[4′-(2-Amino-benzoimidazol-1-yl)-5-chloro-biphenyl-2-yl]-acetamide

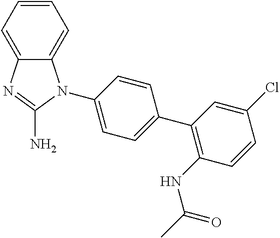 [SMILES: CC(=O)Nc1ccc(Cl)cc1-c1ccc(-n2c(N)nc3ccccc32)cc1]

Intermediate A (557 mg, 1.933 mmol) was dissolved in DME (10 mL) and 2-acetamido-5-chlorophenyl boronic acid pinacol ester (628 mg, 2.126 mmol), Pd(Ph3P)4 (112 mg, 0.097 mmol) and Na2CO3 2M in water (4833 μl, 9.67 mmol) were added. The resulting reaction mixture was stirred at 150° C. for 17 minutes under microwave irradiation. The medium was diluted with MeOH (3 mL) and then passed through a pad of diatomaceous earth which was then washed with EtOAc (3×10 mL). The combined organic phases were evaporated to dryness to furnish the crude title compound, 880 mg (80% purity) which was used without further purification. HPLC/MS (Method A) t_{R} 1.20 minute, M+H 377.1-379.1.

### IV Biology

The efficacy of the compounds of formula x as inhibitors of IGF1-R and InsR tyrosine kinase activity can be demonstrated as follows:

BaF3-Tel-IGF1-R and BaF3-InsR are BaF3 murine proB-cell lymphoma cell derivatives [the BaF3 cell line (also termed Ba/F3) is available from the German Collection of Microorganisms and Cell Cultures (DSMZ), Braunschweig, Germany] that have been rendered IL-3-independent by stable transduction with kinase-activating fusions between human TEL (aa 1-452) and the kinase domain of IGF-1R (aa 976-1367) linked by a Ser-Arg-linker (Boulay et al, Cancer Res 68, 3743-3751, 2008), and a fusion between human TEL (aa 1-337) and the kinase domain (aa 1015-1382) of the Insulin receptor (Melnick J S et al, Proc Natl Acad Sci USA103, 3153-3158, 2006), respectively. Cells are cultured in RPMI-1640 (Animed #1-14F01-1) supplemented with 2% L-glutamine (Animed #5-10K50-H) and 10% fetal calf serum (FCS, Animed #2-01F16-I). Wild-type, untransfected BaF3 cells are maintained in above medium plus 10 U/ml IL-3 (mouse Interleukin-3, Roche #1380745 or Invitrogen # PMC0035) and are used to identify non-selective, generally growth-inhibitory compounds. Cells (1.5×10⁴ cells per well) are seeded in 190 μl fresh medium into 96-well plates. 10 μl 20× compound solutions are added. As internal control, the kinase inhibitor PKC412 is routinely used. Control cells treated with DMSO (0.1% final concentration) serve as growth reference (set as 100% growth). In addition, a plate blank value is routinely determined in a well containing only 100 μl of medium and no cells. IC₅₀ determinations are performed based on eight 3-fold serial dilutions of the test compound, starting at 10 μM. Following incubation of the cells for 48 h at 37° C. and 5% CO₂, the effect of inhibitors on cell viability is assessed by the resazurin sodium salt dye reduction assay (commercially known as AlamarBlue assay) basically as previously described (O'Brien J. et al., Eur. J. Biochem. 267: 5421-5426, 2000). Briefly, 20 μl dye solution is added per well and the plates incubated for 6 h at 37° C. and 5% CO₂. Thereafter, fluorescence is measured using a Saphirell 96-well plate reader (TECAN, Mannedorf, Switzerland) with the following settings: Excitation 544 nm and Emission 590 nm.

For data analysis, the plate blank value is subtracted from all data points. The effect of a particular test compound concentration on cell proliferation and viability is expressed as percentage of the blank-corrected reading obtained for cells treated with vehicle only, which is set as 100%. IC₅₀ values can be determined using XLfit (V4.2), applying standard four parameter logistic model #205 (IDBS, Guilford, UK) or other common curve-fitting software.

To achieve a higher throughput, the cell viability assay can also be performed in a 384-well format. Briefly, 4'500 freshly diluted cells are seeded in 54 μl/well into 384-well plates using a liquid dispenser. 6 μl 10× compound solution is added to the cell plate. As internal control, the kinase inhibitor PKC412 is routinely used. Control cells treated with DMSO (0.1% final concentration) serve as growth reference (set as 100% growth). In addition, a plate blank value is routinely determined in a well containing only 60 μl of medium and no cells. Dose-response effects are determined by 3-fold serial compound dilutions, starting at 10 μM. Following incubation of the cells for 48 h at 37° C. and 5% CO₂, the effect of compounds on cell proliferation/viability is assessed by addition of 6 μl resazurin sodium salt dye solution per well. Following incubation for an additional 6 hrs at 37° C. and 5% CO₂, fluorescence is measured using an Infiniti M1000 microplate reader (TECAN, Mannedorf, Switzerland) with excitation and emission wavelengths set at 544 and 590 nm, respectively. For data analysis, the plate blank value is subtracted from all data points. IC₅₀ values can be determined by four parameter logistic fitting as described above.

| | IC50* | IC50** |
|---|---|---|
| Ex. | [nmol/L] | [nmol/L] |
| 1 | 66 | |
| 2 | 86 | |
| 3 | 617 | |
| 3.1 | 935 | |
| 3.10 | 413 | |
| 3.11 | 523 | |
| 3.12 | 908 | |
| 3.13 | 882 | |
| 3.15 | 619 | |
| 3.14 | 802 | |
| 3.16 | 984 | |
| 3.17 | 538 | |
| 3.18 | 661 | |
| 3.19 | 379 | |
| 3.2 | 956 | |
| 3.20 | 457 | |
| 3.21 | 915 | |
| 3.22 | 629 | |
| 3.23 | 770 | |
| 3.24 | 364 | |
| 3.25 | 316 | |
| 3.3 | 623 | |
| 3.4 | 675 | |
| 3.5 | 1114 | |
| 3.6 | 887 | |
| 3.7 | 973 | |
| 3.8 | 574 | |
| 3.9 | 504 | |
| 4 | 909 | |
| 5 | 159 | |
| 5.1 | 193 | |
| 5.10 | 596 | |
| 5.11 | 950 | |
| 5.12 | 869 | |
| 5.13 | 631 | |
| 5.14 | | 1090 |
| 5.15 | 276 | |
| 5.16 | 407 | |
| 5.17 | 385 | |
| 5.18 | 112 | |
| 5.19 | 40 | |
| 5.2 | 79 | |
| 5.20 | 64 | |
| 5.3 | 349 | |
| 5.4 | 151 | |
| 5.5 | 476 | |
| 5.6 | 721 | |
| 5.7 | 150 | |
| 5.8 | 139 | |
| 5.9 | 299 | |
| 6 | 199 | |
| 7 | 976 | |
| 7.1 | 278 | |
| 7.2 | 600 | |
| 7.3 | 737 | |
| 7.4 | 1350 | |
| 7.5 | 483 | |
| 7.6 | 2553 | |
| 8 | 24 | |
| 9 | 38 | |
| 10 | 492 | |
| 11 | 66 | |
| 12 | 259 | |
| 13 | 562 | |
| 14 | 71 | |
| 15 | 41 | |
| 16 | 142 | |
| 17 | 104 | |
| 18 | 309 | |
| 19 | 42 | |
| 20 | 68 | |
| 21 | 425 | |
| 22 | | 680 |
| 23 | | 119 |
| 24 | 20 | |
| 25 | 13 | |
| 25.1 | 24 | |
| 25.2 | 555 | |
| 25.3 | 13 | |
| 25.4 | 8 | |
| 25.5 | 26 | |
| 25.6 | | 35 |
| 26 | 28 | |
| 27 | 112 | |
| 28 | 733 | |
| 29 | 175 | |
| 30 | | 200 |
| 31 | | 712 |
| 32 | 220 | |
| 33 | 480 | |
| 34 | 202 | |
| 35 | 794 | |
| 36 | 37 | |
| 37 | 8 | |
| 38 | 191 | |
| 40 | 16 | |
| 40.1 | 13 | |
| 40.2 | 32 | |
| 41 | 120 | |
| 43 | 767 | |
| 43.1 | 489 | |
| 43.2 | 628 | |
| 43.3 | 732 | |
| 44 | 166 | |
| 44.1 | 73 | |
| 45 | 173 | |
| 45.1 | 130 | |
| 45.2 | 135 | |
| 46 | 18 | |
| 46.1 | <4.572 | |
| 46.2 | 26 | |
| 46.3 | 26 | |
| 46.4 | 65 | |
| 46.5 | 284 | |
| 47 | 92 | |
| 47.1 | 55 | |
| 47.2 | 65 | |
| 47.3 | 349 | |
| 47.4 | 496 | |
| 47.5 | 536 | |
| 47.6 | 734 | |
| 47.7 | 787 | |
| 47.8 | 1052 | |
| 47.9 | 1074 | |
| 48 | 38 | |
| 48.1 | 14 | |
| 48.2 | <4.5 | |
| 49 | 277 | |
| 50 | 38 | 29 |
| 50.1 | 27 | |
| 50.2 | <4.5 | |
| 50.3 | 146 | |
| 50.4 | | 396 |
| 50.5 | | 195 |
| 50.6 | | 476 |
| 51 | 27 | |
| 51.1 | 28 | |
| 51.2 | 20 | |
| 51.3 | 55 | |
| 52 | 36 | |
| 53 | 31 | |
| 68 | 577 | |
| 68.1 | 946 | |
| 68.2 | 1086 | |
| 68.3 | 383 | |
| 69 | 24 | |
| 70 | 71 | |
| 71 | 46 | |
| 72 | 146 | |
| 73 | 18 | |
| 74 | | 16 |
| 75 | | 61 |
| 76 | | 381 |

*96-well plate format;
**84-well plateformat

### V Pharmaceutical Formulations

### Tablets

Tablets comprising 50 mg of the compounds of formula (I) described in the examples and having the following composition are prepared in customary manner:

Composition: active ingredient 50 mg; wheat starch 150 mg; lactose 125 mg; colloidal silicic acid 12.5 mg; talc 22.5 mg; magnesium stearate 2.5 mg; Total: 362.5 mg.

Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained. The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets weighing 145 mg and having a breaking notch.

### Soft Capsules

5000 soft gelatin capsules comprising each 50 mg of active ingredient, for example one of the compounds of formula (I) described in Examples 1 to 88, are prepared in customary manner:

Composition: active ingredient 250 g; Lauroglykol 2 litres

Preparation: The pulverized active ingredient is suspended in Lauroglykol® (propylene glycol laurate, Gattefossé S.A., Saint Priest, France) and ground in a wet pulverizer to a particle size of approx. 1 to 3 pm. 0.419 g portions of the mixture are then dispensed into soft gelatin capsules using a capsule-filling machine.

1. A compound of formula (I) 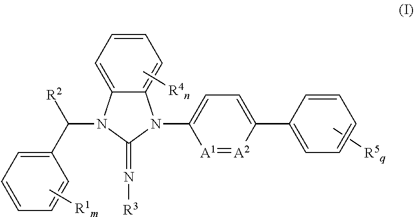
or a salt thereof, wherein
m represents 0, 1, 2, 3 or 4;
n represents 0, 1, 2, 3 or 4;
q represents 0, 1, 2, 3, 4 or 5;
A¹ represents N or CR⁶;
A² represents N or CR⁷;
R¹ represents halogen, C₁₋₇alkyl, C₁₋₇alkyoxy, halo-C₁₋₇alkyl or halo-C₁₋₇alkyoxy; or
R¹ represents, provided two substituents R¹ are in vicinal position, together with the carbon atoms to which they are attached a cyclic moiety, said moiety being (a) saturated or partly saturated, (b) contains 5-8 ring forming atoms, (c) contains 0-3 nitrogen atoms, 0-2 oxygen atoms, and 0-2 sulfur atoms, and (d) is unsubstituted or substituted, the substituents being selected from the group consisting of halogen, C₁₋₇alkyl, C₁₋₇alkyoxy, halo-C₁₋₇alkyl and halo-C₁₋₇alkyoxy;
R² represents hydrogen, halogen, C₁₋₇alkyl or halo-C₁₋₇alkyl;
R³ represents hydrogen, C₁₋₇alkyl, halo-C₁₋₇alkyl, C₁₋₇alkyl-carbonyl-C₀₋₇alkyl, halo-C₁₋₇alkyl-carbonyl-C₀₋₇alkyl, C₁₋₇alkoxy-carbonyl-C₀₋₇alkyl, halo-C₁₋₇alkoxy-carbonyl-C₀₋₇alkyl, C₃₋₆cycloalkyl, or halo-C₃₋₆cycloalkyl;
R⁴ represents halogen, C₁₋₇alkyl, C₁₋₇alkoxy, halo-C₁₋₇alkyl or halo-C₁₋₇alkoxy;
R⁵ represents a substituent different from hydrogen, said substituent (a) having 1-50 atoms selected from the group consisting of hydrogen, carbon, halogen and hetero atoms and (b) being bound via a single bond; or
R⁵ represents, provided two substituents R⁵ are in vicinal position, together with the carbon atoms to which they are attached a cyclic moiety, said moiety being (a) saturated or partly saturated, (b) contains 5-8 ring forming atoms, (c) contains 0-3 nitrogen atoms, 0-2 oxygen atoms, 0-2 sulfur atoms, (d) is unsubstituted or substituted by 1, 2 or 3 substituents, (e) said substituent having 1-50 atoms selected from the group consisting of hydrogen, carbon, halogen and hetero atoms, (f) said substituent being bound via a single bond or double bond;
R⁶ represents, hydrogen, hydroxy, halogen, C₁₋₇alkyl, C₁₋₇alkyoxy, halo-C₁₋₇alkyl or halo-C₁₋₇alkyoxy;
R⁷ represents, hydrogen, hydroxy, halogen, C₁₋₇alkyl, C₁₋₇alkyoxy, halo-C₁₋₇alkyl or halo-C₁₋₇alkyoxy.

2. The compound according to claim 1, or a salt thereof, depicted by formula (I-1)

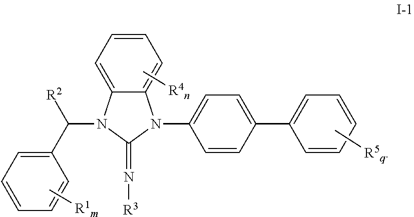

3. The compound according to claim 1, or a salt thereof, depicted by formula (I-2) 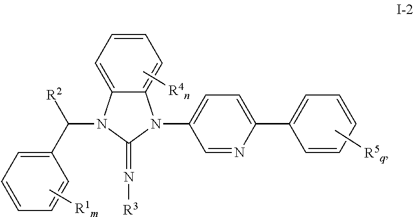
or depicted by formula (I-3) 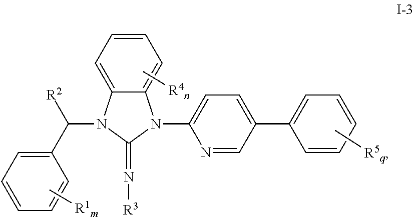
or depicted by formula (I-4)

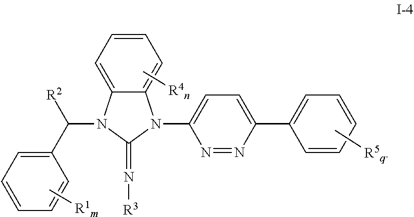

4. The compound according to claim 1, or a salt thereof, depicted by formula (I-5) 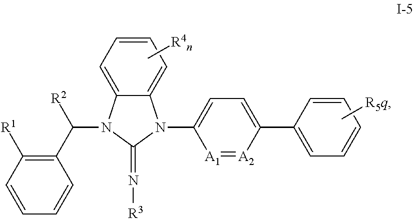
or depicted by formula (I-6) 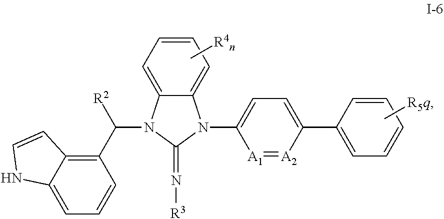
or depicted by formula (I-7) 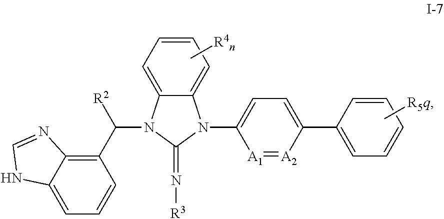
or depicted by formula (I-8)

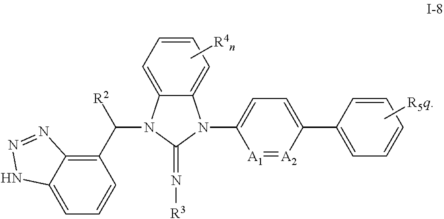

5. The compound according to claim 1, or a salt thereof, depicted by formula (I-9) 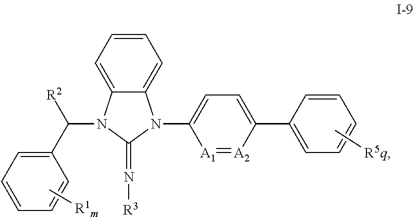
or depicted by formula (I-10)

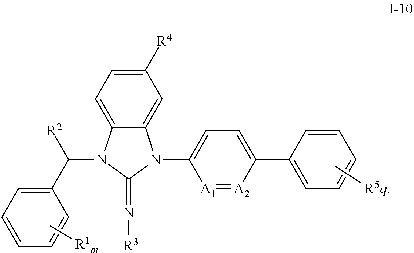

6. The compound according to claim 1, or a salt thereof, wherein
R⁵ represents a group —X′-R⁵′ in which
X′ represents either a single bond or a linker selected from the group consisting of 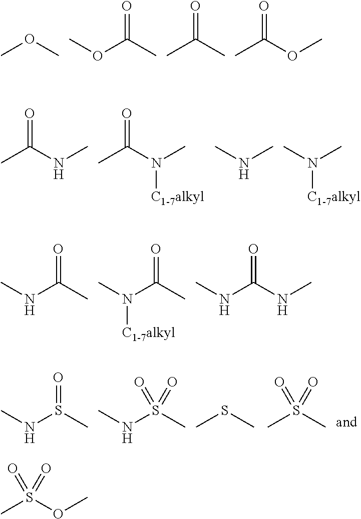
and
R⁵′ represents hydroxy, halo, cyano, carboxy, aminocarbonyl, amino, or optionally substituted C₁₋₇alkyl, optionally substituted C₃₋₁₂cycloalkyl, optionally substituted C₆₋₂₀aryl, optionally substituted heterocyclyl having 3-24 ring atoms, or optionally substituted heteroaryl having 5-14 ring atoms, the optional substituents being selected from the group consisting of hydroxy, halo, cyano, carboxy, amino-carbonyl, amino, C₁₋₇alkylamino, di-(C₁₋₇alkyl)amino, C₁₋₇alkyl, and C₁₋₇alkoxy, phenyl.

7. The compound according to claim 1, or a salt thereof, wherein
R⁵ represents, together with the phenyl ring, an unsubstituted or substituted indolyl, benzimidazolyl, benztriazolyl, the substituents being selected from the group consisting of hydroxy, halo, cyano, carboxy, amino-carbonyl, amino, C₁₋₅alkylamino, di (C₁₋₅alkyl)amino, C₁₋₅alkyl, C₁₋₄alkoxy, phenyl.

8. The compound according to claim 1, or a salt thereof, wherein
R⁵ represents methyl, methoxy, acetylamino, chloro, cyano, or trifluoromethyl.

9. The compound according to claim 1, or a salt thereof, wherein
q represents 2 and the substituents R⁵ being located in the 2- and 5-position or
q represents 1 and the substituent R⁵ being located in the 2- or 3-position.

10. The compound according to claim 1, or a salt thereof, wherein
R′ represents halogen or
R′ represents, together with the phenyl ring, an unsubstituted or substituted indolyl, isoindolyl, indazolyl, benzimidazolyl, benztriazolyl, chinolinyl, isochinnolinyl, cinnolinyl, phtalazinyl, chinazolinyl, chinoxalinyl, naphtalenyl, tetrahydro-naphtalenyl, indenyl, or dihydro-indenyl, the substituents being selected from the group consisting of halogen.

11. The compound according to claim 1, or a salt thereof, wherein
R³ represents hydrogen, C₁₋₄alkyl which is optionally substituted by halo or C₁₋₄ alkyloxy-carbonyl, C₃₋₆cycloalkyl which is optionally substituted by halo.

12. A pharmaceutical composition comprising a therapeutically effective amount of a compound of formula (I) according to claim 1, or a pharmaceutically acceptable salt thereof, and one or more pharmaceutically acceptable carriers

13. The composition of claim 12 further comprising one or more therapeutically active agents, selected from antiproliferative agents.

14-18. (canceled)

19. A method of modulating IGF-1R activity in a subject, comprising the step of administering to a subject a therapeutically effective amount of a compound of formula (I) according to claim 1, or a pharmaceutically acceptable salt thereof.

20. A method for the treatment of an IGF-1R mediated disorder or disease comprising the step of administering to a subject a therapeutically effective amount of a compound of formula (I) according to claim 1, or a pharmaceutically acceptable salt thereof.

21. The method of claim 20, wherein said IGF-1R mediated disorder or disease is selected from the group consisting of multiple myeloma, neuroblastoma, synovial, hepatocellular, Ewing's Sarcoma, and adrenocotical carcinoma or is a solid tumor selected from the group consisting of osteosarcoma, melanoma, tumor of breast, renal, prostate, colorectal, thyroid, ovarian, pancreatic, lung, uterine and gastrointestinal tumor or is acute lung injury or pulmonary fibrosis.
